(19) United States
(12) Patent Application Publication
(10) Pub. No.: US 2008/0287376 A1
(43) Pub. Date: Nov. 20, 2008

(54) Ketolide Derivatives as Antibacterial Agents

(75) Inventors: Biswajit Das, Haryana (IN); Mohammad Salman, Princeton, NJ (US); Atul Kashinath Hajare, Maharashtra (IN); Ramadass Venkataramanan, New Delhi (IN); Rita Katoch, Chandigarh (IN); Rajesh Kumar, Delhi (IN); Gobind Singh Kapkoti, Navi Mumbai (IN); Anjan Chakrabarti, Haryana (IN); Anish Bandyopadhyay, Haryana (IN); Santosh Haribhau Kurhade, Maharashtra (IN); Yogesh Baban Surase, Maharashtra (IN); Ashok Rattan, Delhi (IN)

Correspondence Address: RANBAXY INC., 600 COLLEGE ROAD EAST, SUITE 2100, PRINCETON, NJ, 08540, US

(73) Assignees: Mohammad SALMAN, Princeton, NJ (US); RANBAXY LABORATORIES LIMITED, Haryana (IN)

(21) Appl. No.: 11/572,619

(22) PCT Filed: Jul. 28, 2005
(86) PCT No.: PCT/US05/27875

§ 371 Date: Jul. 18, 2008

(30) Foreign Application Priority Data
Jul. 28, 2004 (IN) 1400/DEL/2004
May 30, 2005 (IN) 1388/DEL/2005

(51) Int. Cl. A61K 31/7048; C07H 17/08; A61P 31/04

(52) U.S. Cl. 514/29; 536/7.1

(57) ABSTRACT

The present invention provides ketolide derivatives, which can be used as antibacterial agents. In particular, compounds described herein can be used for treating or preventing conditions caused by or contributed to by Gram-positive, Gram-negative or anaerobic bacteria, more particularly against, for example, Staphylococci, Streptococci, Enterococci, Haemophilus, Moraxalla spp. Chlamydia spp., Mycoplasm, Legionella spp., Mycobacterium, Helicobacter, Clostridium, Bacteroides, Corynebaclerium, Bacillus or Enterobactericeae. Also provided are processes for preparing such ketolide derivatives, pharmaceutical compositions thereof, and methods of treating bacterial infections.

### FIELD OF INVENTION

The present invention provides ketolide derivatives, which can be used as antibacterial agents. Compounds described herein can be used for treating or preventing conditions caused by or contributed to by Gram-positive, Gram-negative or anaerobic bacteria, more particularly against, for example, Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus, Enterobactericeae or any combination thereof. Also provided are processes for preparing compounds described herein, pharmaceutical compositions containing compounds described herein, and methods of treating bacterial infections.

### BACKGROUND OF THE INVENTION

First generation macrolides erythromycin A and early derivatives are characterized by bacteriostatic or bactericidal activity for most Gram-positive bacteria, atypical pathogens, and many community-acquired respiratory infections and in patients with penicillin allergy. However, erythromycin A causes numerous drug-drug interactions, has relatively poor absorption, poor local tolerance, loses its antibacterial activity under acidic conditions by degradation and the degraded products are known to be responsible for undesired side effects (Itoh, Z et ah, Am. J. Physiol, 1984, 247:688; Omura, S et ah, J. Med. Chem., 1987, 30:1943). Various erythromycin A derivatives have been prepared to overcome the acid instability and other problems associated with it.

Roxithromycin, clarithromycin and azithromycin were developed to address the limitation of erythromycin A. Both clarithromycin and azithromycin were found to be important drugs in the treatment and prophylaxis of atypical mycobacterial infections in patients with HIV.

Macrolides were found to be effective drugs in the treatment of many respiratory tract infections. However, increasing resistance among S. pneumoniae has prompted the search for new compounds that retain favorable safety profiles, retain a spectrum of activity and are confined to respiratory pathogens. Consequently, numerous investigators have prepared chemical derivatives of erythromycin A in an attempt to obtain analogs having modified or improved profiles of antibiotic activity. Ketolides exhibit greater efficacy and safety, have broader spectrum of activities, and are particularly effective against resistant pathogens; hence, ketolides have been developed as next generation macrolides.

U.S. Pat. No. 5,635,485 discloses erythromycin compounds that are reportedly useful in the treatment of bacterial infections in warm-blooded animals.

U.S. Pat. No. 5,866,549 discloses semi-synthetic macrolides reportedly having antibacterial activity, as well as 6-O-substituted erythromycin ketolide derivatives and a method of treating bacterial infections.

U.S. Pat. Nos. 6,458,771 and 6,399,582 and PCT Publication Nos. WO 00/62783 and WO 00/44761 disclose ketolide antibacterials that are reportedly useful in treating bacterial and protozoal infections and in treating other conditions involving gastric motility.

U.S. Pat. No. 5,747,467 discloses erythromycin and antibacterial composition and a method of treating bacterial infection in warm-blooded animals.

U.S. Pat. No. 6,433,151 discloses erythromycin derivatives and their use as medicament for treating infections caused by particular Gram-positive bacteria, namely Haemophilus influenzae, and Moraxalla spp.

U.S. Pat. No. 6,472,372 discloses 6-O-carbamoyl ketolide antibacterials and methods of treating bacterial infections.

U.S. Patent Application Nos. 2002/0115621 and 2003/0013665 disclose macrolide compounds that are said to be useful as antibacterial and antiprotozoal agents in mammals, including man, as well in fish and birds.

Other ketolide compounds have also been reported. A. Denis and A. Bonnefoy, Drugs of the Future, 26(10):975-84 (2001), Champney W. S., et al, Current Microbiology, 42:203-10 (2001).

However, there remains a need for novel ketolide derivatives, which can be used as antibacterial agents on a wide variety of Gram-positive, Gram-negative or anaerobic bacteria.

### SUMMARY OF THE INVENTION

The present invention provides ketolide derivatives, which can be used in the treatment or prevention of bacterial infection, and processes for the synthesis of these compounds.

Pharmaceutically acceptable salts, pharmaceutically acceptable solvates, enantiomers, diastereomers, polymorphs of these compounds having same type of activity are also provided.

Pharmaceutical compositions containing the described compounds together with pharmaceutically acceptable carriers, excipients or diluents, which can be used for the treatment of bacterial infection.

Thus in one aspect, provided herein are compounds having the structure of Formula I,

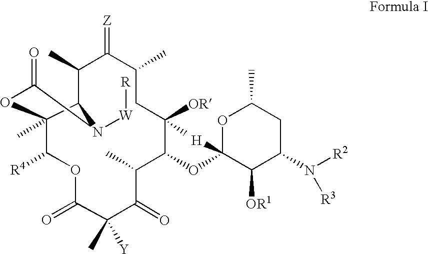

pharmaceutically acceptable salts, pharmaceutically acceptable solvates, enantiomers, diastereomers or polymorphs thereof, wherein
R¹ can be hydrogen or a hydroxyl protecting group;
R² and R³ can independently be alkyl, alkenyl, alkynyl, cycloalkyl, aryl, heterocycle, aralkyl, (heterocycle)alkyl or COR¹¹, wherein R¹¹ can be hydrogen, alkyl or aralkyl, with the proviso that R² and R₃ are not simultaneously methyl;
W can be alkenyl, -G(CH₂)_{q}J-, —CR⁹R¹⁰, —NR⁹— or —SO₂, wherein
   q can be an integer of from 2 to 6;
   G can be no atom, —CO, —CS or —SO₂;
   R⁹ and R¹⁰ can independently be hydrogen or alkyl; and
   J can be no atom, —CR⁹R¹⁰ or N(R¹²)(CH₂)ₘ, wherein m can be an integer of from 0 to 6; R⁹ and R¹⁰ can be the same as defined earlier; and R¹² can be hydrogen, alkyl, alkylene, alkynyl, COR⁸ or —(CH₂)ₘ—R⁸, wherein R⁸ can be alkyl, aryl or heterocycle;
R can be aryl or heterocycle;
R⁴ can be alkyl, alkenyl or alkynyl;
R′ can be alkyl or —(CH₂)ᵣ—U, wherein r can be an integer of from 1 to 4 and U can be alkenyl or alkynyl; and
Y can be halogen, cyano or alkyl; Z can be oxygen, sulfur or NOR¹¹, wherein R¹¹ can be the same as defined earlier.

These compounds can include one or more of the following embodiments. For example, R can be heterocycle; R can be methyl; R can be alkyl (except methyl), alkenyl, cycloalkyl or COR¹¹; W can be -G(CH₂)_{q}J- or CR⁹R¹⁰, wherein G, q, J, R⁹, R¹⁰ and R¹¹ can be the same as defined above. In another embodiment, R¹ can be hydrogen or a hydroxy protecting group (wherein the hydroxy protecting can be benzoyl, tetrahydropyranyl or a trialkylsilylether); R² can be CH₃; R³ can be C₂H₅, —CH₂—CH═CH₂ or —CH₂CH₂F; W can be —(CH₂)₄-J-, wherein J can be CH₂ or (CH₂)₀₋₁—N(CO)—Rₐ; and R can be:

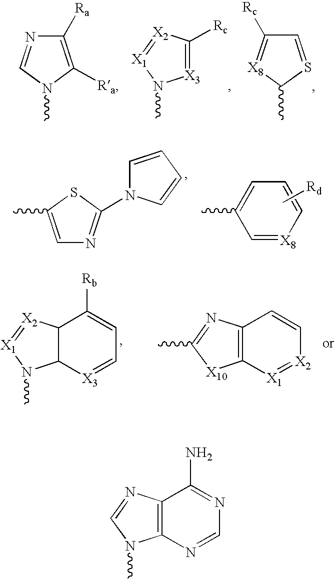

wherein X₁-X₃ can independently be CH or N; X₄-X₈ can independently be CH, CR₄ or N; X₉ can be O, S, N, NH or CH; X₁₀ can be NH or S; Rₐ can be thienyl, furyl, pyrazolyl, oxazolyl, tetrazolyl, imidazolyl, pyridinyl, fluoropyridinyl, chloropyridinyl, aminopyridinyl, pyrazinyl, pyrimidinyl, aminopyrimidinyl, quinolinyl, pyrrolo-pyridyl, pyrrolo-thiazolyl or optionally substituted phenyl; R′a can be hydrogen or furyl; R_{b} can be hydrogen or amino; R_{c} can be hydrogen, thienyl, furyl, pyrazolyl, pyrazinyl, pyridinyl, aminopyridinyl, pyrimidinyl, pyrrolyl, imidazolyl or optionally substituted phenyl; and R_{d} can be thienyl, pyrazolyl, imidazolyl, triazolyl, pyrrolyl or tetrahydrofuryl.

In another aspect, provided herein are compounds selected from:
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((3-(imidazol-1-yl)-propyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(1H-imidazo[4,5-b]-pyridin-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-imidazo[4,5-b]-pyridin-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(imidazol-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((3-(imidazol-1-yl)-propyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((3-(imidazol-1-yl)-propyl)-imino)]erythromycin A,
2-α-Fluoro-5-O-(3′-N-desmethyl-3′-N-propyl)-desosaminyl-11,12-dideoxy-3-O-decladinosyl-6-O-methyl-3-oxo-12,11-[oxycarbonyl-((3-(imidazol-1-yl)-propyl)-imino)]erythromycin A,
2-α-Fluoro-5-O-(3′-N-desmethyl-3′-N-propyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(pyrrolo-[2,3-b]pyridin-1-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(pyrrolo-[2,3-b]pyridin-1-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-n-propyl)-desosaminyl-11,12-dideoxy-3-O-decladinosyl-6-O-methyl-3-oxo-12,11-[oxycarbonyl-((4-(imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(pyrrolo-[2,3-b]pyridin-1-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(1H-imidazo[4,5-b]-pyridin-1-yl)-butyl)-imino)]erythromycin A,
2-α-Fluoro-5-O-(3′-N-desmethyl-3′-N-ethyl-desosaminyl-11,12-dideoxy-3-O-decladinosyl-6-O-methyl-3-oxo-12,11-[oxycarbonyl-((4-(purin-9-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(3H-imidazo[4,5-b]-pyridin-3-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyridin-3-yl)-imidazol-1-yl)-butyl)-imino)]erythromycin A,
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′-methyl-N′-quinolin-4-ylmethyl)-2-aminoethyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′-methyl-N′-pyridine-3-ylmethyl)-2-aminoethyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′-methyl-N-pyridine-4-ylmethyl)-2-aminoethyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′-methyl-N′-pyridine-2-ylmethyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′-methyl-N′-pyridine-2-ylmethyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′-methyl-N′-pyridine-3-ylmethyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′-methyl-N′-pyridine-4-ylmethyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′-methyl-N′-quinoline-4-ylmethyl)-2-aminoethyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′-pyridine-3-ylmethyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′-acetyl-N′-pyridine-3-ylmethyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′-pyridine-3-ylmethyl)-2-aminoethyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′-acetyl-N′-pyridine-3-ylmethyl)-2-aminoethyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′,N′-di-pyridine-3-yl-methyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′,N′-di-pyridine-3-yl-methyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(quinolin-3-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(quinolin-8-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(pyridin-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(pyridin-3-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(pyridin-4-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(pyridin-2-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(pyridin-4-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(pyridin-3-yl)-pentyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(pyridin-3-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(indol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-phenylimidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-nitroindol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(1H-imidazo[4,5-c]pyridin-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(benzotriazol-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-imidazo[4,5-c]pyridin-3-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(4-phenylimidazol-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(benzotriazol-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(benzotriazol-2-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(1H)-imdazo[4,5-b]pyridin-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(indol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(benzotriazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(3H-imidazo[4,5-b]pyridin-3-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(4-phenylimidazol-1-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(benzotriazol-2-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(indol-1-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(3H-imidazo[4,5-b]pyridin-3-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(5-nitroindol-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(5-nitroindol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-fluoroindol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(benzotriazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-methylbenzoimidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-ethylbenzoimidazol-1-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(benzotriazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(2-ethylbenzoimidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-((4-(pyridin-3-yl)-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(2-methylbenzoimidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(1H-imidazo[4,5-b]pyridin-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(3H-imidazo[4,5-b]pyridin-3-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(5-nitroindol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(2-(pyridin-3-ylmethyl)-benzoimidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-(pyridin-3-yl-methyl)-benzoimidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(2-phenylimidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(5-fluoroindol-1-yl)-butyl)-imino)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((4-(5,6-dimethyl-benzoimidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5,6-dimethylbenzoimidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(3H-imidazo[4,5-c]pyridin-3-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(2-methyl-3H-imidazo[4,5-b]pyridin-3-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-methyl-3H-imidazo[4,5-b]pyridin-3-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(1H-imidazo[4,5-c]pyridin-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-amino-9H-purin-9-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-phenylimidazol-1-yl)-butyl)-imino)]erythromycin A,
2-α-Fluoro-5-O-(3′-N-desmethyl-3′-N-acetyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-cyclopropyl methyl)-desosaminyl-11,12-dideoxy-3-O-decladinosyl-6-O-methyl-3-oxo-12,11-[oxycarbonyl-((4-(3H-imidazo[4,5-b]pyridin-3-yl)-butyl)-imino)]erythromycin A,
2-α-Fluoro-5-O-(3′-N-desmethyl-3′-N-cyclopropyl methyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-cyclopropylmethyl)-desosaminyl-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(benzotriazol-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(4-phenyl-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-trifluoromethyl-benzoimidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4,5-diphenyl-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4,5-diphenyl-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4-phenyl-imidazol-1-yl)-butyl)-imino)]erythromycin A,
2-α-Fluoro-5-O-(3′-N-desmethyl-3′-N-cyclopropylmethyl)-desosaminyl-11,12-dideoxy-3-O-decladinosyl-6-O-methyl-3-oxo-12,11-[oxycarbonyl-((4-(4-phenyl-imidazol-1-yl)-butyl)-imino)]erythromycin A,
2-α-Fluoro-5-O-(3′-N-desmethyl-3′-N-cyclopropyl methyl)-desosaminyl-11,12-dideoxy-3-O-decladinosyl-6-O-methyl-3-oxo-12,11-[oxycarbonyl-((4-(benzotriazol-1-yl)-butyl)-imino)]erythromycin A,
2-α-Fluoro-5-O-(3′-N-desmethyl-3′-N-cyclopropyl methyl)-desosaminyl-11,12-dideoxy-3-O-decladinosyl-6-O-methyl-3-oxo-12,11-[oxycarbonyl-((4-(4-(pyridin-3-yl)-imidazol-1-yl)-butyl)-imino)]erythromycin A,
2-α-Fluoro-5-O-(3′-N-desmethyl-3′-N-acetyl)-desosaminyl-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A,
2-α-Fluoro-5-O-(3′-N-desmethyl-3′-N-acetyl)-desosaminyl-11,12-dideoxy-3-O-decladinosyl-6-O-methyl-3-oxo-12,11-[oxycarbonyl-((4-(4-phenyl-imidazol-1-yl)-butyl)-imino)]erythromycin A,
2-α-Fluoro-5-O-(3′-N-desmethyl-3′-N-acetyl)-desosaminyl-11,12-dideoxy-3-O-decladinosyl-6-O-methyl-3-oxo-12,11-[oxycarbonyl-((4-(1H-imidazo[4,5-b]pyridin-1-yl)-butyl)-imino)]erythromycin A,
2-α-Fluoro-5-O-(3′-N-desmethyl-3′-N-acetyl)-desosaminyl-11,12-dideoxy-3-O-decladinosyl-6-O-methyl-3-oxo-12,11-[oxycarbonyl-((4-(3H-imidazo[4,5-b]pyridin-3-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(3,5-diphenyl)-pyrazol-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(4-(pyridin-3-yl)-imidazol-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(1H-imidazo[4,5-b]pyridin-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3,5-diphenyl)-pyrazol-1-yl)-butyl)-imino)]erythromycin A,
2-α-Fluoro-5-O-(3′-N-desmethyl-3′-N-cyclopropyl methyl)-desosaminyl-11,12-dideoxy-3-O-decladinosyl-6-O-methyl-3-oxo-12,11-[oxycarbonyl-((4-(1H-imidazo[4,5-b]pyridin-1-yl)-butyl)-imino)]erythromycin A,
2-α-Fluoro-5-O-(3′-N-desmethyl-3′-N-acetyl)-desosaminyl-11,12-dideoxy-3-O-decladinosyl-6-O-methyl-3-oxo-12,11-[oxycarbonyl-((4-(4-(pyridin-3-yl)-imidazo-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(4-(2,4-difluoro-phenyl)-imidazol-1-yl)-butyl)-imino)]erythromycin A,
2-α-Fluoro-5-O-(3′-N-desmethyl-3′-N-2-ethyl)-desosaminyl-11,12-dideoxy-3-O-decladinosyl-6-O-methyl-3-oxo-12,11-[oxycarbonyl-((4-(4-(2,4-difluorophenyl)-imidazol-1-yl)-butyl)-imino)]erythromycin A,
2-α-Fluoro-5-O-(3′-N-desmethyl-3′-N-2-allyl)-desosaminyl-11,12-dideoxy-3-O-decladinosyl-6-O-methyl-3-oxo-12,11-[oxycarbonyl-((4-(4-(2,4-difluoro-phenyl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(furan-3-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(furan-3-yl)-imidazol-1-yl)-butyl)-imino)]erythromycin A hydrochloride salt,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-((4-phenyl-2H-pyrazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyridin-3-yl)-1H-imidazol-1-yl)-pentyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyridin-4-yl)-1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4-(pyridin-4-yl)-1H-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-phenyl-2H-pyrazol-1-yl)-butyl)-imino)erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(1H-indazol-2-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(1H-indazol-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyridin-3-yl)-2H-pyrazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(1H-indazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyrazin-2-yl)-1H-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(thiophen-3-yl)-1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4-(thiophen-3-yl)-1H-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(quinolin-3-yl)-1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(1H-indazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyrimin-5-yl)-1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4-(pyrimin-5-yl)-1H-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(furan-2-yl)-1H-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(thiophen-2-yl)-1H-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(4-chloro-phenyl) 1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4-(4-chloro-phenyl)-1H-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyridin-2-yl)-1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4-(pyridin-2-yl)-1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((9-(4-amino-butyl)-9H-purin-6-yl)-imino)]erythromycin A,
2-α-Fluoro-5-O-(3′-N-desmethyl-3′-N-2-allyl)-desosaminyl-11,12-dideoxy-3-O-decladinosyl-6-O-methyl-3-oxo-12,11-[oxycarbonyl-((4-(4-(pyridin-3-yl)-1H-pyrazol-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(4-(4-chloro-phenyl)-1H-imidazol-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(4-(pyridin-2-yl)-1H-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-imidazo[4,5-b]pyridine-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(tetrazol-1-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(1H-benzoimidazol-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(6-fluoro-pyridin-3-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-imidazo[4,5-c]pyridine-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-([1,2,4]trizol-1-yl)-phenyl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(imidazol-1-yl)-phenyl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(6-chloro-pyridin-3-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(3-(1-(4-aminobutyl)-1H-imidazol-4-yl)-phenyl)imino]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyridin-3-yl)-thiazol-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(3-(2-(4-aminobutyl)-thiazol-4-yl)-phenyl)imino]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyrazol-1-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-([1,4′]-bipyrazolyl-1′-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(imidazol-1-yl)-pyrazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyrazol-1-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-([3,3′]bithiophenyl-5-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-([2,3′]bithiophenyl-5′-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(furan-2-yl)-thiophen-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(oxazol-5-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3-(pyrrol-1-yl)-[1,2,4]triazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-(thiophen-2-yl)-tetrazol-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(thiophen-3-yl)-pyrazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(furan-3-yl)-pyrazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(furan-2-yl)-pyrazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-phenyl-tetrazol-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-(4-methoxy-phenyl)-tetrazol-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-(furan-3-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-pyrazol-1-yl-pyridin-3-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyridin-3-yl)-pyrazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-thiophen-2-yl-pyridin-3-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-phenyl-thiophen-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(N-(4-amino-butyl)-N-thiazol-2-yl-nicotinimido)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(N-(4-amino-butyl)-N-thiazol-2-yl-nicotinimido)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(6-pyrrol-1-yl-pyridin-3-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-pyrrol-1-yl-pyridin-3-yl)-butyl)-imino)]erythromycin A,
2-∝-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-pyrrol-1-yl-thiazol-5-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-imidazol-1-yl-pyridin-3-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(tetrahydro-furan-2-yl)-pyridin-3-yl]-butyl)imino]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-tolyl-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-aminophenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-methyl-3-aminophenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(imidazol-1yl)-phenyl)imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N,N-dicyclopropyl-amino)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N,N-dimethyl-amino)phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(tetrazol-1yl)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(2-(pyrrol-1yl)-thiozol-5-yl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-trifluoromethyl-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(N-(thiazol-2-yl)-benzimido)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(2-amino-pyrimidin-5-yl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-(pyrrol-1-yl)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(pyrrol-1yl)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-acetyl-amino)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-isopropyl-amino)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-nitrophenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-benzoyl-amino)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3,4-dimethyl-phenyl)-imidazol-1-yl)-butyl)-imino)]erythromycin A,
5-O-(3′-N-desmethyl-3′-N-ethyl)-11,12-dideoxy-3-O-decladinosyl-6-O-methyl-3-oxo-12,11-[oxycarbonyl-((4-(4-Pyridin-3-yl-thiazol-2-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-fluorophenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-methoxy-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(N-(benzthiazol-2-yl)-benzimido)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(N-(thiazol-2-yl)-nicotinamido)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-methyl-amino)-phenyl)-imidazol-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(2-aminopyridin-4-yl)-imidazol-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(2-aminopyridin-5-yl)-2H-tetrazol-5-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3,4-difluorophenyl)-imidazol-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(2-chloropyridin-4-yl)-imidazol-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(6-aminopyridin-3-yl)-imidazol-1-yl)-butyl)-imino)]erythromycin A, or
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(5-(2-Chloro-pyridin-4-yl)-tetrazol-2-yl])-butyl)-imino)]erythromycin A,
   or pharmaceutically acceptable salts, pharmaceutically acceptable solvates, enantiomers, diastereomers or polymorphs thereof.

In yet another aspect, provided herein are pharmaceutical compositions comprising therapeutically effective amounts of one or more compounds of compounds described herein together with one or more pharmaceutically acceptable carriers, excipients, or diluents.

In another aspect, provided herein are methods for treating or preventing conditions caused by or contributed to by bacterial infections comprising administering to a mammal in need thereof therapeutically effective amounts of one or more compounds of compounds described herein.

The methods may include one or more of the following embodiments. For example, the condition can be selected from community acquired pneumonia, upper or lower respiratory tract infections, skin or soft tissue infections, hospital acquired lung infections, hospital acquired bone or joint infections, mastitis, catether infection, foreign body, prosthesis infections or peptic ulcer disease. In another embodiment, the bacterial infection can be caused by Gram-positive, Gram-negative or anaerobic bacteria.

In yet another embodiment, the Gram-positive, Gram-negative or anaerobic bacteria can be selected from Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus or Enterobactericeae. In a preferred embodiment, the bacterium is cocci. In another preferred embodiment, the cocci is drug resistant.

In another aspect, provided herein are processes for preparing compounds of Formula XIII,

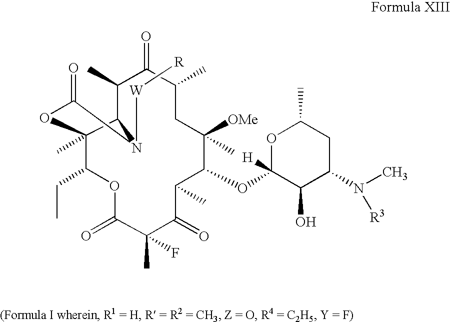

or their pharmaceutically acceptable salts, pharmaceutically acceptable solvates, enantiomers, diastereomers or polymorphs, wherein R³ can be alkyl, alkenyl, alkynyl, cycloalkyl, aryl, heterocycle, aralkyl, (heterocycle)alkyl or COR¹¹ (wherein R¹¹ can be hydrogen, alkyl or aralkyl, with the proviso that R³ is not methyl); W can be alkenyl, -G(CH₂)_{q}J-, —CR⁹R¹⁰, —NR⁹— or —SO₂ (wherein q can be an integer of from 2 to 6; G can be no atom, —CO, —CS or —SO₂; R⁹ and R¹⁰ can independently be hydrogen or alkyl; and J can be no atom, —CR⁹R¹⁰ or N(R¹²)(CH₂)ₘ {wherein m can be an integer of from 0 to 6; R⁹ and R¹⁰ can be the same as defined earlier; and R¹² can be hydrogen, alkyl, alkylene, alkynyl, COR⁸ or —(CH₂)ₘ—R⁸, wherein R⁸ can be alkyl, aryl or heterocycle}); and R can be aryl or heterocycle;

which processes comprise the steps of:
(a) hydrolyzing clarithromycin of Formula II,

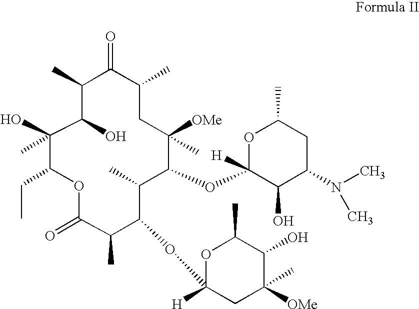

to form a compound of Formula III,

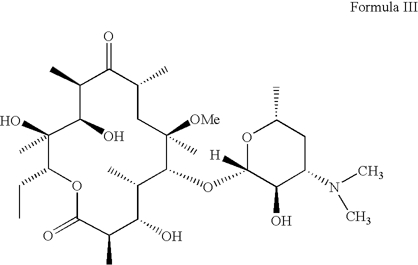

(b) protecting the compound of Formula III by reacting with one or more reagents of Formula R¹₂O or R¹X (wherein X can be halogen) to form a compound of Formula IV,

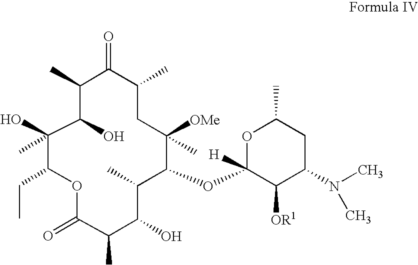

(c) desmethylating the compound of Formula IV at the 3′-N-dimethyl group to form a compound of Formula V,

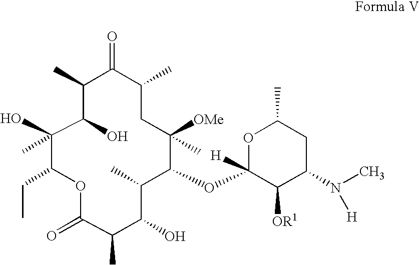

(d) alkylating the compound of Formula V with one or more reagents of Formula R³CHO, R₂³CO or R³X (wherein X can be halogen) to form a compound of Formula VI (wherein R³ can be the same as defined earlier),

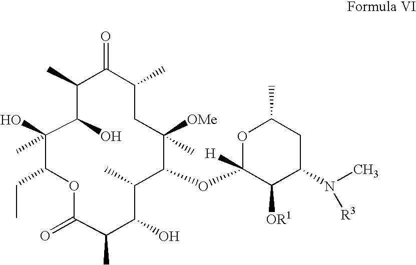

(e) converting the compound of Formula VI with one or more suitable reagents to form a compound of Formula VII,

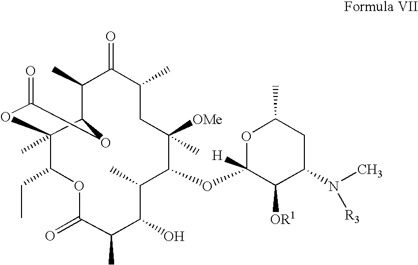

(f) reacting the compound of Formula VII with one or more organic bases to form a compound of Formula VIII,

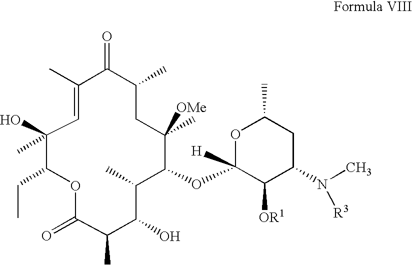

(g) oxidizing the compound of Formula VIII to form a compound of Formula IX,

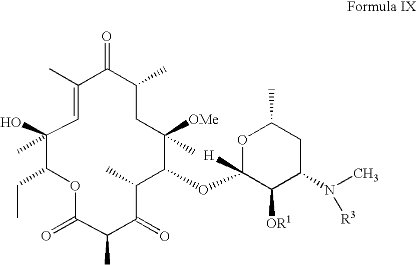

(h) reacting the compound of Formula IX with N,N′-carbonyldimidazole to form a compound of Formula X,

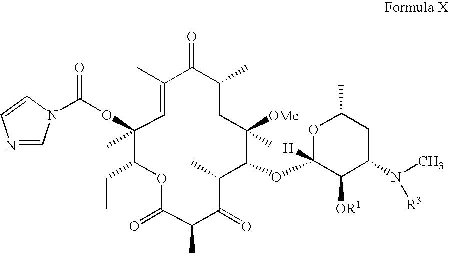

(i) reacting the compound of Formula X with a compound of Formula R—W—NH₂ to form a compound of Formula XI (wherein W and R can be the same as defined earlier),

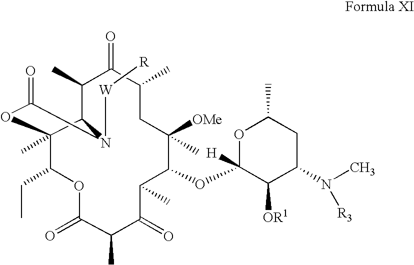

(j) fluorinating the compound of Formula XI to form a compound of Formula XII, and

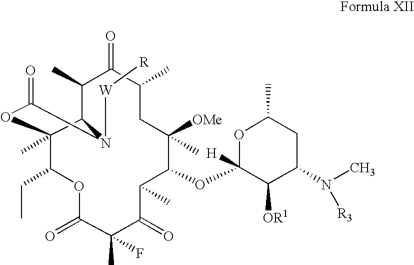

(k) deprotecting the compound of Formula XII to form a compound of Formula XIII.

In another aspect, provided herein are processes for preparing compounds of Formula XIII,

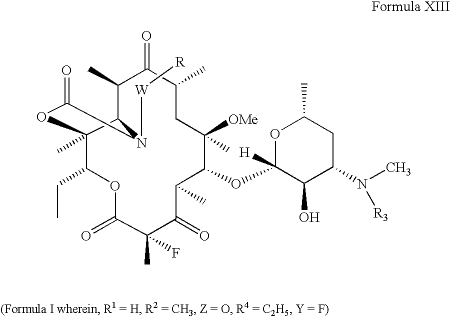

or their pharmaceutically acceptable salts, pharmaceutically acceptable solvates, prodrugs, stereoisomers or polymorphs, wherein R can be alkyl, alkenyl, alkynyl, cycloalkyl, aryl, heterocycle, aralkyl, (heterocycle)alkyl or COR¹¹ (wherein R¹¹ can be hydrogen, alkyl or aralkyl, with the proviso that R³ is not methyl); W can be alkenyl, -G(CH₂)_{q}J-, —CR⁹R¹⁰, —NR⁹— or —SO₂ (wherein q can be an integer of from 2 to 6; G can be no atom, —CO, —CS or —SO₂; R⁹ and R¹⁰ can independently be hydrogen or alkyl; and J can be no atom, —CR⁹R¹⁰ or N(R¹²)(CH₂)ₘ {wherein m can be an integer of from 0 to 6; R⁹ and R¹⁰ can be the same as defined earlier; and R¹² can be hydrogen, alkyl, alkylene, alkynyl, COR⁸ or —(CH₂)ₘ—R⁸, wherein R⁸ can be alkyl, aryl or heterocycle}); and R can be aryl or heterocycle;

which processes comprise the steps of:
(a) reacting a compound of Formula IV with a suitable reagent,

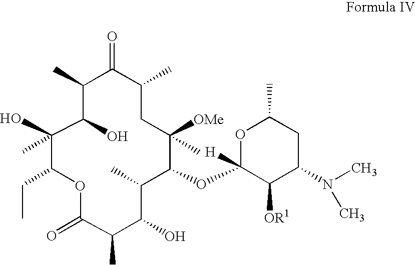

to form a compound of Formula XIV,

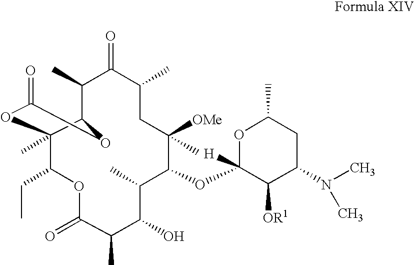

(b) reacting the compound of Formula XIV with one or more organic bases to form a compound of Formula XV,

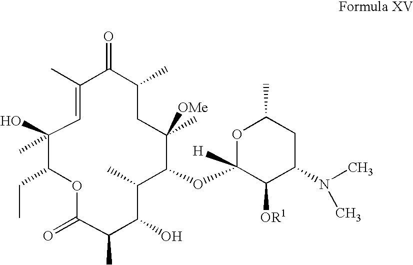

(c) oxidizing the compound of Formula XV to form a compound of Formula XVI,

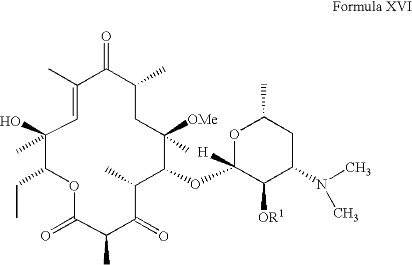

(d) desmethylating the compound of Formula XVI at the 3′-N-dimethyl group to form a compound of Formula XVII,

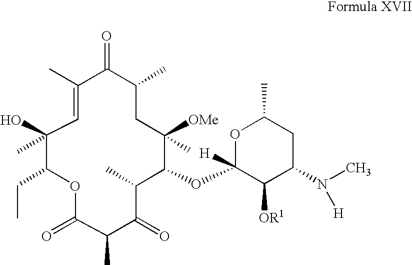

(e) alkylating the compound of Formula XVII with one or more reagents of Formula R³CHO, R³₂CO or R³X (wherein X can be halogen) to form a compound of Formula IX,

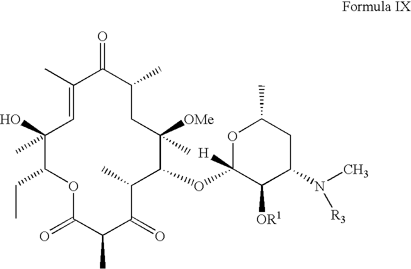

(f) fluorinating the compound of Formula IX to form a compound of Formula XVIII,

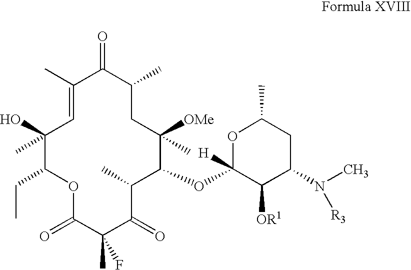

(g) reacting the compound of Formula XVIII with N,N′-carbonyldiimidazol to form a compound of Formula XIX,

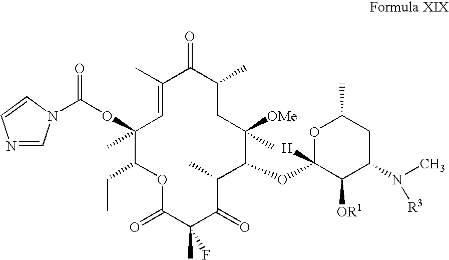

(h) reacting the compound of Formula XIX with a compound of Formula R—W—NH₂ to form a compound of Formula XII, and

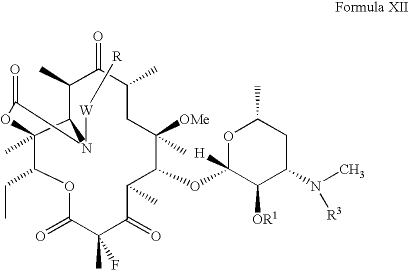

(i) deprotecting the compound of Formula XII to form a compound of Formula XIII.

In another aspect, provided herein are processes for preparing compounds of Formula XIII,

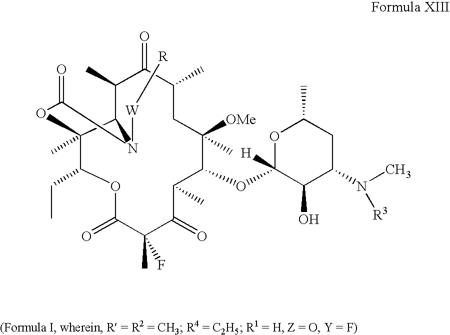

or their pharmaceutically acceptable salts, pharmaceutically acceptable solvates, stereoisomers, prodrugs or polymorphs, wherein R³ can be alkyl, alkenyl, alkynyl, cycloalkyl, aryl, heterocycle, aralkyl, (heterocycle)alkyl or COR¹¹, wherein R¹¹ can be hydrogen, alkyl or aralkyl, with the proviso that R³ is not methyl; W can be alkenyl, -G(CH₂)_{q}J-, —CR⁹R¹⁰, —NR⁹— or —SO₂ (wherein q can be an integer of from 2 to 6; G can be no atom, —CO, —CS or —SO₂; R⁹ and R¹⁰ can independently be hydrogen or alkyl; and J can be no atom, —CR⁹R¹⁰ or N(R¹²)(CH₂)ₘ {wherein m can be an integer of from 0 to 6; R⁹ and R¹⁰ can be the same as defined earlier; and R¹² can be hydrogen, alkyl, alkylene, alkynyl, COR⁸ or —(CH₂)ₘ—R⁸, wherein R⁸ can be alkyl, aryl or heterocycle}); and R can be aryl or heterocycle;

which processes comprises the steps of:
(a) fluorinating the compound of Formula XVI,

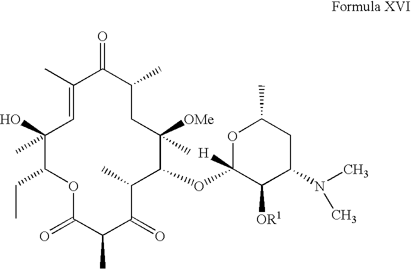

to form a compound of Formula XX,

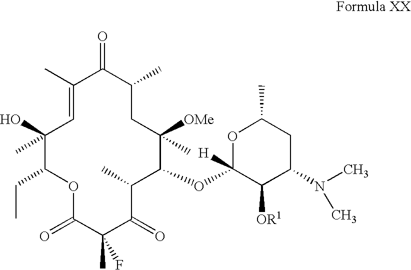

(b) reacting the compound of Formula XX with N,N′-carbanoyldiimidazole to form a compound of Formula XXI,

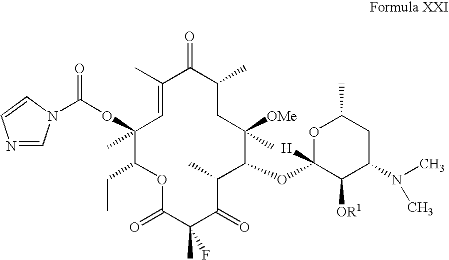

(c) reacting the compound of Formula XXI with a compound of Formula R—W—NF₂ to form a compound of Formula XXII,

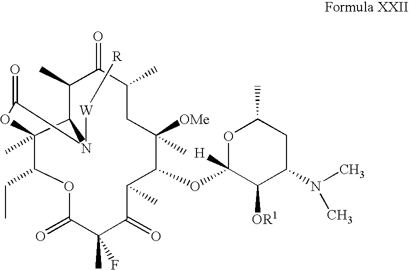

(d) deprotecting the compound of Formula XXII to form a compound of Formula XXIII,

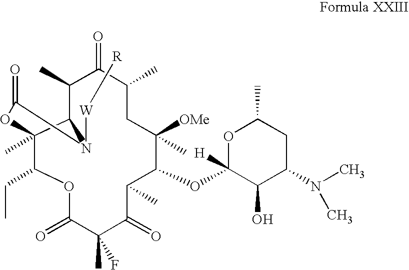

(e) desmethylating the compound of Formula XXIII at the 3′-N-dimethyl group to form a compound of Formula XXIV, and

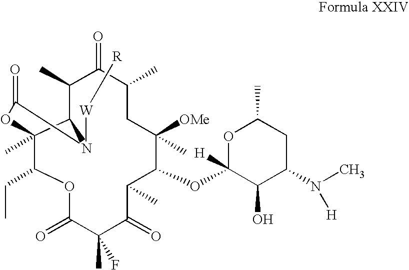

(f) alkylating the compound of Formula XXIV with one or more reagents of Formula R³CHO, R³₂CO or R³X (wherein X can be halogen) to form a compound of Formula XIII.

In another aspect, provided herein are processes for preparing compounds of Formula XIII,

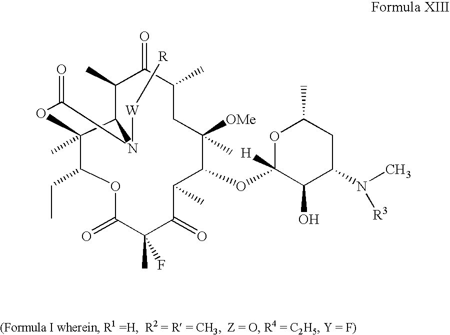

or their pharmaceutically acceptable salts, pharmaceutically acceptable solvates, stereoisomers, prodrugs or polymorphs, wherein R³ can be alkyl, alkenyl, alkynyl, cycloalkyl, aryl, heterocycle, aralkyl, (heterocycle)alkyl or COR¹¹, wherein R¹¹ can be hydrogen, alkyl or aralkyl, with the proviso that R³ is not methyl; W can be alkenyl, -G(CH₂)_{q}J-, —CR⁹R¹⁰, —NR⁹— or —SO₂ (wherein q can be an integer of from 2 to 6; G can be no atom, —CO, —CS or —SO₂; R⁹ and R¹⁰ can independently be hydrogen or alkyl; and J can be no atom, —CR⁹R¹⁰ or N(R¹²)(CH₂)ₘ {wherein m can be an integer of from 0 to 6; R⁹ and R¹⁰ can be the same as defined earlier; and R¹² can be hydrogen, alkyl, alkylene, alkynyl, COR⁸ or —(CH₂)ₘ—R⁸, wherein R⁸ can be alkyl, aryl or heterocycle}); and R can be aryl or heterocycle;

which processes comprise the steps of:
(a) deprotecting the compound of Formula XX (wherein R¹ can be COPh),

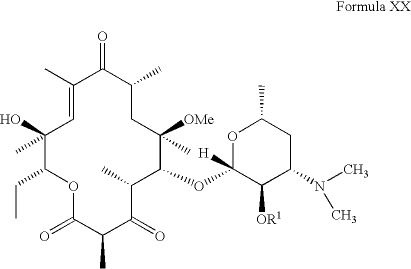

to form a compound of Formula XXV,

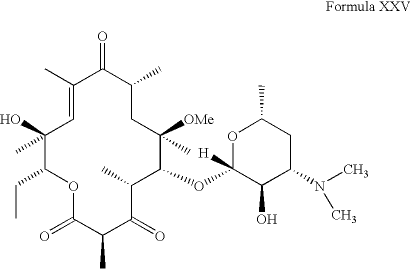

(b) desmethylating the compound of Formula XXV at 3-N′-dimethyl group to form a compound of Formula XXVI,

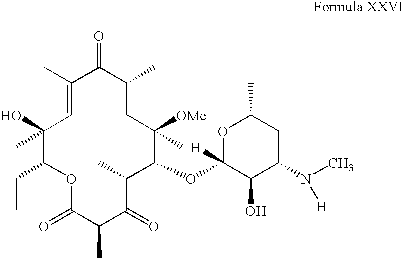

(c) alkylating the compound of Formula XXVI with one or more reagents of Formula R³CHO, R³₂CHO or R³X (wherein X can be halogen) to form a compound of Formula XXVII,

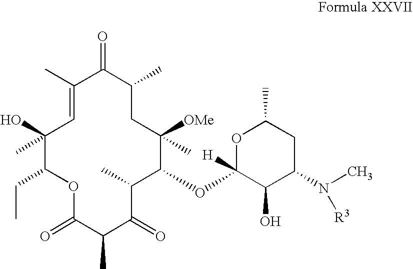

(d) protecting the compound of Formula XXVII with one or more reagents of Formula R¹₂O or R¹X (wherein X can be halogen) to form a compound of Formula XVIII (wherein R¹ can be COCH₃),

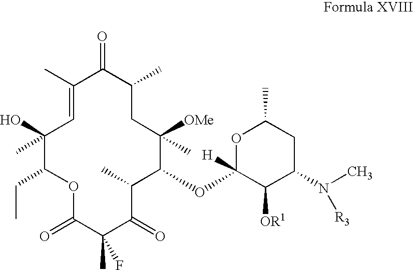

(e) reacting the compound of Formula XVII with N,N′-carbonyldiimidazole to form a compound of Formula XIX,

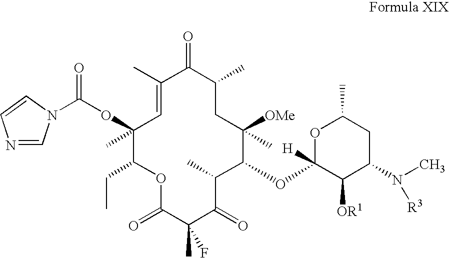

(f) reacting the compound of Formula XIX with a compound of Formula R—W—NH₂ to form a compound of Formula XII, and

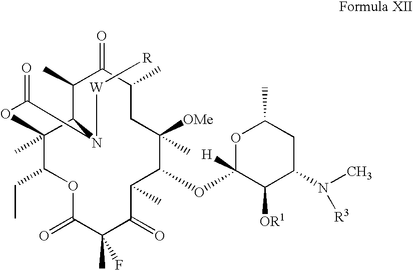

(g) deprotecting the compound of Formula XII to form a compound of Formula XIII.

### DETAILED DESCRIPTION OF THE INVENTION

In accordance with one aspect, provided herein are compounds having the structure of Formula I,

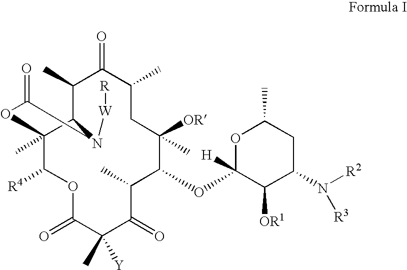

their pharmaceutically acceptable salts, pharmaceutically acceptable solvates, stereoisomers, prodrugs or polymorphs wherein,
R¹ can be hydrogen or hydroxyl protecting group;
R² and R³ can independently be alkyl, alkenyl, alkynyl, cycloalkyl, aryl, heterocycle, aralkyl, (heterocycle)alkyl or COR¹¹ (wherein R¹¹ can be hydrogen, alkyl or aralkyl), with the proviso that R² and R³ are not simultaneously methyl;
W can be alkenyl, -G(CH₂)_{q}J-, —CR⁹R¹⁰, —NR⁹— or —SO₂, wherein
   q can be an integer of from 2 to 6; G can be no atom, —CO, —CS or —SO₂; R⁹ and R¹⁰ can independently be hydrogen or alkyl; J can be no atom, —CR⁹R¹⁰or N(R¹²)(CH₂)ₘ, wherein
      m can be an integer of from 0 to 6; R⁹ and R¹⁰ are the same as defined earlier; R¹² can be hydrogen, alkyl, alkylene, alkynyl, COR⁸ or —(CH₂)ₘ—R⁸, wherein
         R⁸ can be alkyl, aryl or heterocycle;
R can be aryl or heterocycle;
R⁴ can be alkyl, alkenyl or alkynyl;
R′ can be alkyl or —(CH₂)ᵣ—U, wherein r can be an integer from 1 to 4 and U can be alkenyl or alkynyl;
Y can be halogen, cyano or alkyl; and
Z can be oxygen, sulfur or NOR¹¹, wherein R¹¹ can be hydrogen, alkyl or aralkyl.

In one embodiment, R can be heterocycle; R² and R³ can be respectively methyl and alkyl (except methyl), alkenyl, cycloalkyl or COR¹¹; W can be -G(CH₂)_{q}J- or CR⁹R¹⁰, wherein G, q, J, R⁹, R¹⁰ and R¹¹ are the same as defined above.

In another embodiment, R¹ can be hydrogen or a hydroxy protecting group, wherein the hydroxy protecting can be benzoyl, tetrahydropyranyl or a trialkylsilylether; R² can be CH₃; R³ can be C₂H₅, —CH₂—CH═CH₂ or —CH₂CH₂F; W can be —(CH₂)₄-J-, wherein J can be CH₂ or (CH₂)₀₋₁—N(CO)—Rₐ; and R can be,

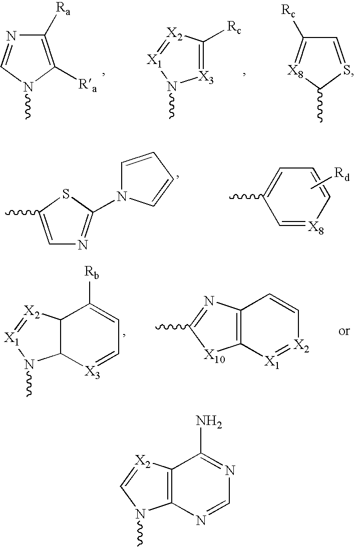

wherein
X₁-X₃ can independently be CH or N; X₄-X₈ can independently be CH, CR₄ or N; X₉ can be O, S, N, NH or CH; X₁₀ can be NH or S; Rₐ can be thienyl, furyl, pyrazolyl, oxazolyl, tetrazolyl, imidazolyl, pyridinyl, fluoropyridinyl, chloropyridinyl, aminopyridinyl, pyrazinyl, pyrimidinyl, aminopyrimidinyl, quinolinyl, pyrrolo-pyridyl, pyrrolo-thiazolyl or optionally substituted phenyl; R′a can be hydrogen or furyl; R_{b} can be hydrogen or amino; R_{c} can be hydrogen, thienyl, furyl, pyrazolyl, pyrazinyl, pyridinyl, aminopyridinyl, pyrimidinyl, pyrrolyl, imidazolyl or optionally substituted phenyl; and R_{d} can be thienyl, pyrazolyl, imidazolyl, triazolyl, pyrrolyl or tetrahydrofuryl.

In accordance with a second aspect, provided herein are methods for treating or preventing a mammal suffering from conditions caused by or contributed to by Gram-positive, Gram-negative or anaerobic bacteria comprising administering to a mammal in need thereof therapeutically effective amounts of one or more compounds or one or more pharmaceutical compositions described herein.

Bacterial infection may be caused by one or more bacteria, for example, Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus or Enterobactericeae.

The conditions treated or prevented may be, for example, community acquired pneumonia, upper and lower respiratory tract infections, skin and soft tissue infections, hospital acquired lung infections or bone and joint infections, or other bacterial infections, for example, mastitis, catether infection, foreign body, prosthesis infections or peptic ulcer disease.

In accordance with a third aspect, provided herein are processes for preparing the described compounds.

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulfinyl, sulfonyl group or —NRₐ—, wherein Rₐ can be hydrogen, alkyl, alkenyl, alkynyl cycloalkyl or aryl. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further (referred herein as “substituted alkyl”) with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl (for R₆-R₉, alkyl is not substituted with aryl), heterocyclyl, heteroaryl, arylthio, thiol, alkylthio, aryloxy, nitro, aminosulfonyl, aminocarbonylamino, —NHC(═O)Rₖ, —NRₚR_{q}, —C(═O)NRₚR_{q}, —NHC(═O)NRₚR_{q}, —C(═O)heteroaryl, C(═O)heterocyclyl, —O—C(═O)NRₚR_{q} {wherein Rₚ and R_{q} are independently selected from alkyl, alkenyl, cycloalkyl, cycloalkenyl, aryl, aralkyl, heterocyclyl, heteroaryl, heterocyclylalkyl, heteroarylalkyl}, nitro, hydroxyamino, alkoxyamino or S(O)ₘR₆₆ (wherein m is an integer from 0-2 and R₆₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —NRₚR_{q}, —C(═O)NRₚR_{q}, —OC(═O)NRₚR_{q}(—NHC(═O)NR_{fp}R_{q} (wherein Rₚ and R_{q} are the same as defined earlier), hydroxy, alkoxy, halogen, CF₃, cyano, and S(O)ₘR₆₆ (wherein m is an integer from 0-2 and R₆₆ are the same as defined earlier); or an alkyl group also may be interrupted by 1-5 atoms of groups independently selected from oxygen, sulfur or —NRₐ— {wherein Rₐ is selected from hydrogen, alkyl, cycloalkyl, alkenyl, cycloalkenyl, alkynyl, aryl, acyl, aralkyl, —C(═O)ORₚ (wherein Rₚ is the same as defined earlier), S(O)ₘR⁶⁶ (wherein m is an integer from 0-2 and R₆₆ is as defined earlier), or —C(═O)NRₚR_{q} (wherein Rₚ and R_{q} are as defined earlier)}. Unless otherwise constrained by the definition, all substituents may be substituted further by 1-3 substituents selected from alkyl, carboxy, carboxyalkyl, —NRₚR_{q}, —C(═O)NRₚR_{q}, —O—C(═O)NRₚR_{q} (wherein Rₚ and R_{q} are the same as defined earlier) hydroxy, alkoxy, halogen, CF₃, cyano, and S(O)ₘR₆₆ (wherein m is an integer from 0-2 and R₆₆ is same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans, or geminal geometry. It can be optionally interrupted by atom(s) or group(s) independently chosen from oxygen, sulfur, phenylene, sulfinyl, sulfonyl and —NRₐ—, wherein Rₐ can be hydrogen, alkyl, alkenyl, alkynyl, cycloalkyl or aryl. In the event that alkenyl is attached to a heteroatom, the double bond cannot be alpha to the heteroatom. Alkenyl groups may be substituted further (referred to herein as “substituted alkenyl”) with one or more substituents selected from alkyl, alkenyl, alkynyl, alkoxy, cycloalkyl, cycloalkenyl, acyl, acylamino, acyloxy, —NHC(═O)Rₚ, —NRₚR_{q}, —C(═O)NRₚR_{q}, —NHC(═O)NRₚR_{q}, —O—C(═O)NRₚR_{q} (wherein Rₚ and R_{q} are the same as defined earlier), alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro, or SO₂R₆₆ (wherein R₆₆ are is same as defined earlier). Unless otherwise constrained by the definition, alkenyl substituents optionally may be substituted further by 1-3 substituents selected from alkyl, carboxy, hydroxy, alkoxy, halogen, —CF₃, cyano, —NRₚR_{q}, —C(═O)NRₚR_{q}, —O—C(═O)NRₚR_{q} (wherein Rₚ and R_{q} are the same as defined earlier) and —SO₂R₆₆ (where R₆₆ is same as defined earlier). Groups such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkynyl,” unless otherwise specified, refers to a monoradical of an unsaturated hydrocarbon, having from 2 to 20 carbon atoms. It can be optionally interrupted by atom(s) or group(s) independently chosen from oxygen, sulfur, phenylene, sulfinyl, sulfonyl and —NRₐ—, where Rₐ can be hydrogen, alkyl, alkenyl, alkynyl, cycloalkyl or aryl. In the event that alkynyl is attached to a heteroatom, the triple bond cannot be alpha to the heteroatom. Alkynyl groups may be substituted further (referred to herein as “substituted alkynyl”) with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)Rₚ, —NRₚR_{q}, —NHC(═O)NRₚR_{q}, —C(═O)NRₚR_{q}, —O—C(═O)NRₚR_{q} (wherein Rₚ and R_{q} are the same as defined earlier), S(O)ₘR₆₆ (wherein m is an integer from 0-2 and R₆₆ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NRₚR_{q}, —C(═O)NRₚR_{q}, —NHC(═O)NRₚR_{q}, —C(═O)NRₚR_{q} (wherein Rₚ and R_{q} are the same as defined earlier), cyano, or S(O)ₘR₆₆ (wherein m is an integer from 0-2 and R₆₆ is same as defined earlier). Groups such as ethynyl, (—C≡CH), propargyl (or propynyl, —CH₂C≡CH), and the like exemplify this term.

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl, and bicyclo[2.2.1]heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NRₚR_{q}, —NHC(═O)NRₚR_{q}, —NHC(═O)Rₚ, —C(═O)NRₚR_{q}, —O—C(═O)NRₚR_{q} (wherein Rₚ and R_{q} are the same as defined earlier), nitro, heterocyclyl, heteroaryl, heterocyclylalkyl, heteroarylalkyl, or S(O)ₘR₆₆ (wherein m is an integer from 0-2 and is same as defined earlier). Unless otherwise constrained by the definition, cycloalkyl substituents optionally may be substituted further by 1-3 substituents selected from alkyl, carboxy, hydroxy, alkoxy, halogen, CF₃, —NRₚR_{q}, —C(═O)NRₚR_{q}, —NHC(═O)NRₚR_{q}, —O—C(═O)NRₚR_{q} (wherein Rₚ and R_{q} are the same as defined earlier), cyano or S(O)ₘR₆₆ (wherein m is an integer from 0-2 and R₆₆ is same as defined earlier). As used herein the term “halogen or halo” refers to fluorine, chlorine, bromine or iodine.

As used herein the term “hydroxyl protected” includes, but is not limited to, acyl, aroyl, alkyl, aryl, butyldiphenylsilyl, methoxymethyl and methylthiomethyl, and the like.

As used herein the term “thio” refers to the group —SH.

The term “alkoxy” denotes the group O-alkyl or O-cycloalkyl, wherein alkyl and cycloalkyl are the same as defined above. Examples of alkoxy include, but are not limited to, methoxy, ethoxy, cyclopentoxy, and the like.

As used herein the term “thioalkyl” refers to —SR⁵ wherein R⁵ is alkyl or cycloalkyl.

As used herein the term “haloalkyl” refers to alkyl of which one or more hydrogen(s) is/are replaced by halogen.

The term “aryl” herein refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₛ (wherein Rₛ is hydrogen, alkyl, alkenyl, cycloalkyl, aralkyl, heterocyclylalkyl, heteroarylalkyl), NHC(═O)Rₚ, —NRₚR_{q}, —C(═O)NRₚR_{q}, —NHC(═O)NRₚR_{q}, —O—C(═O)NRₚR_{q} (wherein Rₚ and R_{q} are the same as defined earlier), S(O)ₘR₆₆ (wherein m is an integer from 0-2 and R₆₆ is same as defined earlier), carboxy, heterocyclyl, heteroaryl, heterocyclylalkyl, heteroarylalkyl or amino carbonyl amino. The aryl group optionally may be fused with a cycloalkyl group, wherein the cycloalkyl group may optionally contain heteroatoms selected from O, N or S. Groups such as phenyl, naphthyl, anthryl, biphenyl, and the like exemplify this term.

The term “aralkyl,” unless otherwise specified, refers to alkyl-aryl linked through an alkyl portion (wherein alkyl is as defined above) and the alkyl portion contains 1-6 carbon atoms and aryl is as defined above. Examples of aralkyl include, but are not limited to, benzyl, napthylmethyl, phenethyl and phenylpropyl, and the like.

The terms “heterocycle” or “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, alkoxy, cycloalkyl, acyl, amino, optionally substituted aryl, alkoxy, alkaryl, cyano, nitro, oxo, carboxy, guanidine, haloalkyl, optionally substituted heterocyclyl, optionally substituted heterocyclylalkyl, heteroaryl, —CORₚ, —O—C(═O)Rₚ, —O—C(═O)ORₚ, —C(═O)NRₚR_{q}, S(O)ₘR₆₆, —O—C(═O)NRₚR_{q}, nitro, —NHC(═O)NRₚR_{q}, —NRₚR_{q} (wherein m, R₆₆, Rₚ and R_{q} are as defined earlier), —NHCORₚ, —NHSO₂Rₚ, and —SO₂NHRₚ, mercapto or thioalkyl.

Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocycles include, but not limited to, azabicyclohexyl, azetidinyl, benzoimidazolyl, 1,4-benzodioxanyl, 1,3-benzodioxolyl, benzoxazolyl, benzothiazolyl, benzothiazinyl, benzotriazolyl, benzoxazinyl, carbaxolyl, dihydrobenzofuryl, dihydroimidazolyl, dihydropyranyl, dihydrofuranyl, dihydroindolyl, dihydroisoxazolyl, dihydropyridinyl, dioxanyl, dioxolanyl, furyl, homopiperidinyl, imidazolyl, imidazolinyl, imidazolidinyl, imidazopyridinyl, indolinyl, indolyl, isoindole 1,3-dione, isoquinolinyl, isothiazolidinyl, isothiazolyl, isoxazolidinyl, isoxazolyl, morpholinyl, napthyridinyl, oxazolidinyl, oxazolyl, phenoxazinyl, phenothiazinyl, piperazinyl, piperidinyl, purinyl, pyrazinyl, pyrazolinyl, pyrazolyl, pyridinyl, pyridyl, pyrimidinyl, pyrrolidinyl, pyrrolinyl, pyrrolyl, pyrrolopyridinyl, quinolinyl, tetrahydrofuranyl, tetrahydropyranyl, tetrazolyl, thiazolidinyl and thiazolyl, and thienyl and the like.

As used herein the term “(heterocyclyl)alkyl” refers to heterocycle which is bonded to an alkylene chain, wherein heterocyclyl and alkyl are the same as defined above. Examples of heterocycle alkyl include, but are not limited to, isothiazolidinyl ethyl, isothiazolyl propyl, pyrazinyl methyl, pyrazolinyl propyl and pyridyl butyl, pyridyl methyl and the like.

As used herein the term “polymorphs” refers to all crystalline forms and amorphous forms of the compounds described herein. In addition, some of the compounds described herein may form solvates with water (i.e., hydrate, hemihydrate or sesquihydrate) or common organic solvents. Such solvates are also encompassed within the scope of this invention.

The phrase “pharmaceutically acceptable salts” denotes salts of the free base, which possess the desired pharmacological activity of the free base and which are neither biologically nor otherwise undesirable. Suitable pharmaceutically acceptable salts may be prepared from an inorganic or organic acid. Example of such inorganic acids include, but not limited to, hydrochloric, hydrobromic, hydroiodic, nitrous (nitrite salt), carbonic, sulfuric, phosphoric acid and like. Appropriate organic acids include, but not limited to, aliphatic, cycloaliphatic, aromatic, heterocyclic, carboxylic and sulfonic classes of organic acids, for example, formic, acetic, propionic, succinic, glycolic, gluconic, lactic, malic, tartaric, citric, ascorbic, glucuronic, maleic, fumeric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, beta-hydroxybutyric, cyclohexylaminosulfonic, galactaric and galacturonic acid and the like.

The term pharmaceutically acceptable carriers” is intended to include non-toxic, inert solid, semi-solid or liquid filler, diluent, encapsulating material or formulation auxiliary of any type.

The compounds of present invention include stereoisomers. The term “stereoisomer” refers to compounds, which have identical chemical composition, but differ with regard to arrangement of the atoms and the groups in space. These include enantiomers, diastereomers, geometrical isomers, atropisomer and comformational isomers. Geometric isomers may occur when a compound contains a double bond or some other feature that gives the molecule a certain amount of structural rigidity. An enantiomer is a stereoisomer of a reference molecule that is the nonsuperimposable mirror image of the reference molecule. A diastereomer is a stereoisomer of a reference molecule that has a shape that is not the mirror image of the reference molecule. An atropisomer is a conformation of a reference compound that converts to the reference compound only slowly on the NMR or laboratory time scale. Conformational isomers (or conformers or rotational isomers or rotamers) are stereoisomers produced by rotation about σ bonds, and are often rapidly interconverting at room temperature. Racemic mixtures are also encompassed within the scope of this invention.

Compounds described herein may be prepared by techniques well known to one of ordinary skill in the art. In addition, compounds described herein may also be prepared by the following reaction sequences as depicted in Schemes I, II, III or IV below.

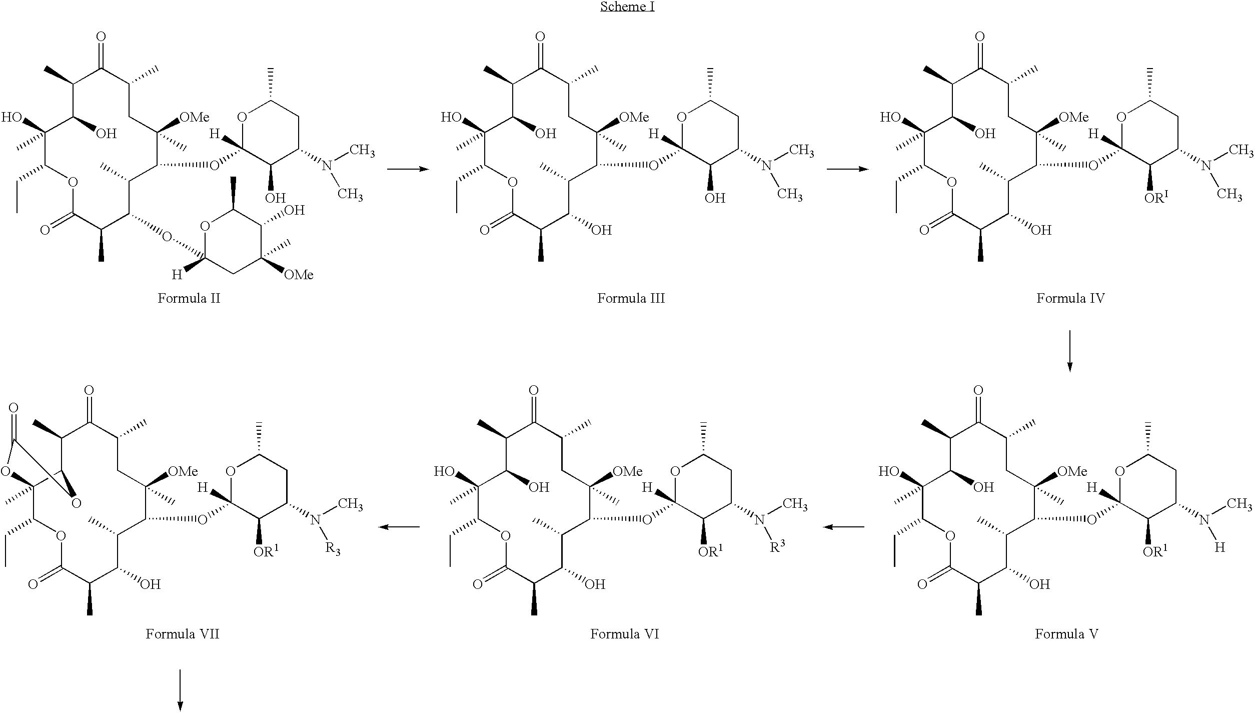

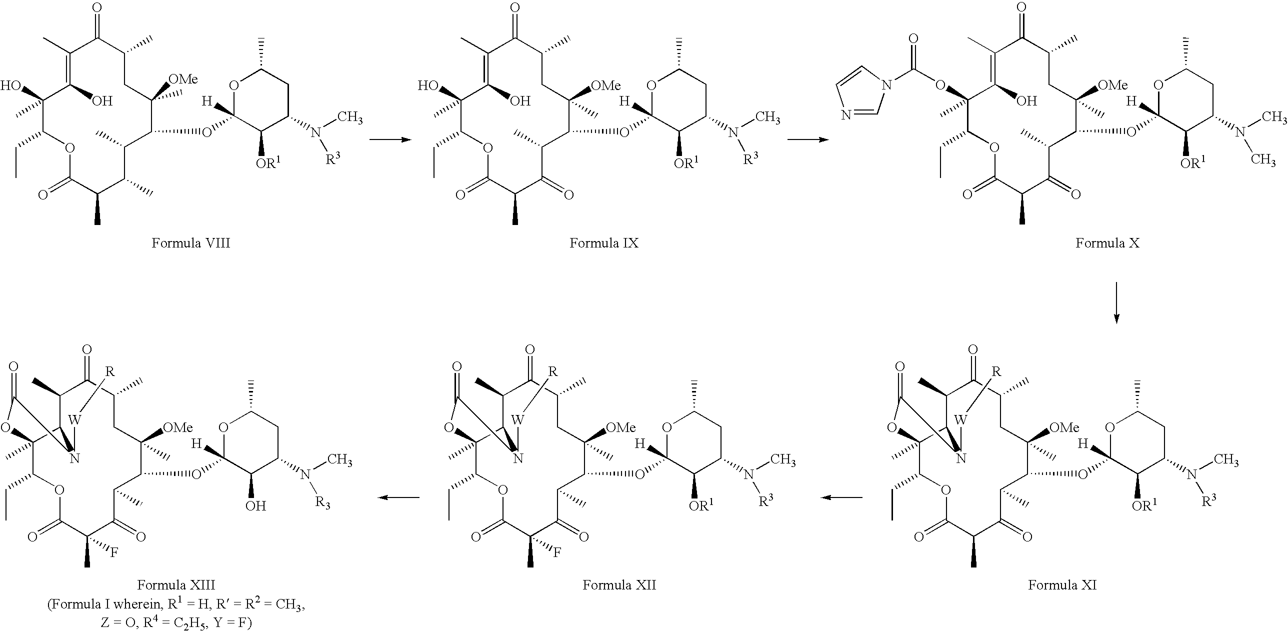

Compounds of Formula XIII can be prepared according to Scheme I. Thus, clarithromycin of Formula II can be hydrolyzed to form a compound of Formula III. The compound of Formula II can be protected by reacting with one or more reagents of Formula R¹₂O or R¹X (wherein X is halogen) to form a compound of Formula IV (wherein R¹═COPh). The compound of Formula IV can be desmethylated at the 3′-N-dimethyl group to form a compound of Formula V. The compound of Formula V can be alkylated by reacting with one or more reagents of Formula R³CHO, R³₂CO or R³X (wherein X is halogen) to form a compound of Formula VI (wherein R³ is the same as defined earlier).

The compound of Formula VI can be reacted with one or more reagents, for example, triphosgene, ethylene dicarbonate or a mixture thereof, to form a compound of Formula VII. The compound of Formula VII can be reacted with one or more organic bases (for example, tetramethyl guanidine, trimethylamine or mixtures thereof) to form a compound of Formula VIII. The compound of Formula VIII can be oxidized to form a compound of Formula IX. The compound of Formula IX can be reacted with N,N′-carbonyldiimidazole to form a compound of Formula X. The compound of Formula X can be reacted with a compound of Formula R—W—NH₂ to form a compound of Formula XI (wherein W and R are the same as defined earlier). The compound of Formula XI can be fluorinated to form a compound of Formula XII. The compound of Formula XII can be deprotected to form a compound of Formula XIII.

Clarithromycin of Formula II can be hydrolyzed in the presence of an inorganic or organic acid, for example, hydrochloric acid, sulfuric acid or dichloroacetic acid.

The compound of Formula III can be hydroxyl protected by reacting with one or more reagents of Formula R¹₂O or R¹X in one or more solvents, for example, dichloromethane, dichloroethane, chloroform, carbon tetrachloride, ethyl acetate or mixtures thereof. The protection reactions can also be carried out in the presence of one or more organic bases, for example, triethylamine, diisopropylethylamine, pyridine, tributylamine, 4-(N-dimethylamino)pyridine or mixtures thereof.

Compounds of Formula IV can be desmethylated in the presence of one or more desmethylating agents, for example, N-iodosuccinimide iodine in acetic acid, diisopropylazodicarboxylate or mixtures thereof. Such desmethylation reactions can also be carried out in one or more solvents, for example, acetonitrile, tetrahydrofuran, dichloromethane, dichloroethane, chloroform, carbon tetrachloride, ethyl acetate or mixtures thereof.

Desmethylation reactions can be quenched in the presence of one or more quenching agents, for example, sodium bisulfite, sodium carbonate, potassium carbonate, cesium carbonate, sodium acetate or mixtures thereof.

Compounds of Formula V can be alkylated with one or more reagents of Formula R³CHO, R³₂CO or R³X in one or more solvents, for example, dimethylformamide, acetonitrile, methanol, acetone, tetrahydrofuran or mixtures thereof. Alkylation reactions can also be carried out in the presence of one or more inorganic or organic bases, for example, sodium hydrogen carbonate, potassium carbonate, cesium carbonate, sodium hydride, pyridine, triethylamine, sodium acetate, sodium thiosulfate, diisopropyl ethylamine or mixtures thereof. Alkylation reactions can also be carried out in the presence of one or more reducing agents (for example, sodium cyanoborohydride, sodium borohydride, sodium triacetoxyborohydride or mixtures there) and in the presence of one or more organic acids (for example, acetic acid or dichloroacetic acid in a solvent, for example, methanol, ethanol, propanol, isopropanol or mixtures thereof).

Compounds of Formula VI can be reacted to form compounds of Formula VII in one or more solvents, for example, chloroform, dichloromethane, carbon tetrachloride, dichloroethane or mixtures thereof. These reactions can also be carried out in the presence of one or more organic bases, for example, triethylamine, diisopropyl ethylamine, pyridine, tributylamine, 4-(N-dimethylamino)pyridine or mixtures thereof.

Compounds of Formula VII can be reacted with one or more organic bases in one or more solvents, for example, dimethylformamide, tetrahydrofuran, dimethylsulfoxide or mixtures thereof.

Compounds of Formula VIII can be oxidized by reacting with one or more oxidizing agents, for example, Dess-Martin periodinane, N-chlorosuccinimide, pyridinium chlorochromate, Swern Oxidation reagent (oxalyl chloride and dimethylsulfoxide), Pfitzner-Moffatt Oxidation reagent (dicyclohexylcarbodiimide and dimethylsulfoxide), Jones Oxidation reagent (chromic acid, aqueous sulfuric acid and acetone), pyridinium dichromate, 1-ethyl-3-(3-dimethylaminopropyl)carbodiimide hydrochloride or mixtures thereof. N-Chlorosuccinamide can be used in combination with dimethyl sulfide and 1-ethyl-3(3-dimethylaminopropyl)carbodiimide hydrochloride can be used in combination with dimethylsulfoxide. Compounds of Formula VIII can also be oxidized in one or more solvents, for example, chloroform, dichloromethane, carbon tetrachloride, dimethylsulfoxide, dichloroethane or mixtures thereof.

Compounds of Formula IX can be reacted with N,N′-carbonyldiimidazole in one or more solvents, for example, dimethylformamide, acetonitrile, tetrahydrofuran or mixtures thereof. This reaction can also be carried out in the presence of one or more inorganic bases, for example, sodium hydrogen carbonate, sodium acetate, sodium thiosulfate, potassium carbonate, cesium carbonate or sodium hydride.

Compounds of Formula X can be reacted with compounds of Formula R—W—NH₂ in one or more solvent systems, for example, acetonitrile/water, dimethylformamide/water, dimethylformamide or combinations thereof.

Compounds of Formula XI can be fluorinated in the presence of one or more fluorinating agents, for example, N-fluorobenzene sulfonimide, selectfluor or mixtures thereof. The fluorination reactions can also be carried out by procedures described in G. Sankar Lai and Syvret R. G., Chem. Rev., 96:1737-1755 (1996). The fluorination reactions can also be carried out in one or more solvents, for example, dimethylformamide, tetrahydrofuran, dimethylsulfoxide or mixtures thereof. Further, the fluorination reactions can also be carried out in the presence of one or more inorganic bases, for example, potassium carbonate, sodium hydride, sodium acetate, sodium thiosulfate, potassium tert-butoxide, sodium tert-butoxide, lithium diisopropylamide, sodium methoxide, sodium ethoxide, potassium methoxide, potassium ethoxide, sodium isopropoxide, potassium isopropoxide, lithium carbonate or mixtures thereof.

Compounds of Formula XII can be deprotected in one or more alcohols, for example, methanol, ethanol, propanol, isopropanol or mixtures thereof.

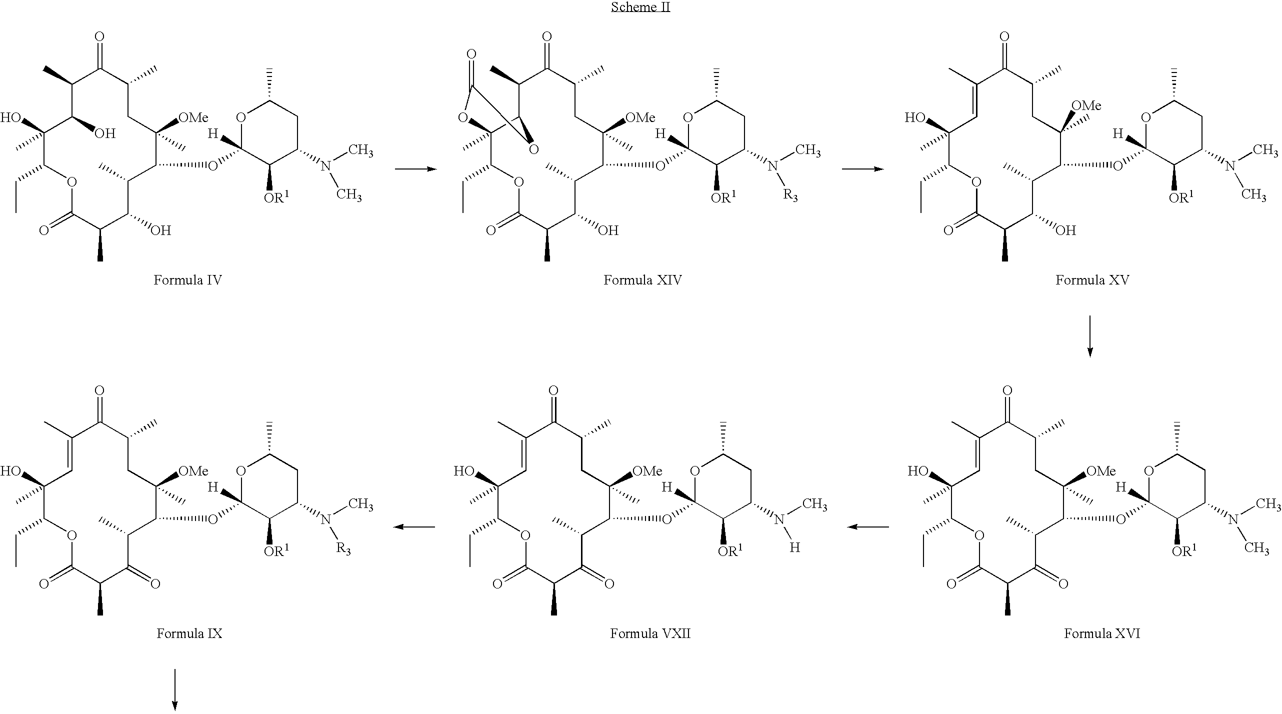

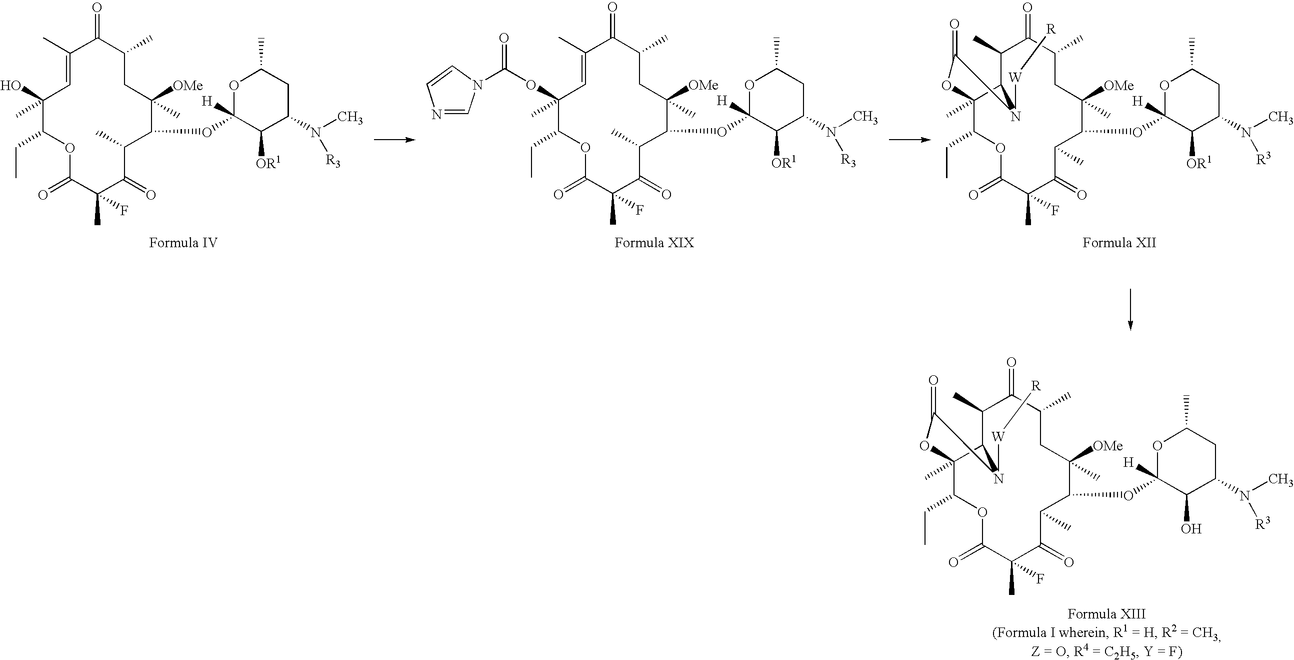

Compounds of Formula XIII can also be prepared according to Scheme II. Thus, compounds of Formula IV can be reacted with one or more reagents, for example, triphosgene, ethylene dicarbonate or mixtures thereof, to form compounds of Formula XIV. Compounds of Formula XIV can be reacted with one or more organic bases, for example, tetramethyl guanidine, trimethyl amine or mixtures thereof, to form compounds of Formula XV. Compounds of Formula XV can be oxidized to form compounds of Formula XVI. Compounds of Formula XVI can be desmethylated at the 3′-N-dimethyl group to form compounds of Formula XVII. Compounds of Formula XVII can be alkylated with one or more reagents of Formula R³CHO, R³₂CO or R³X (wherein X is a halogen) to form compounds of Formula IX (wherein R³ is the same as defined earlier). Compounds of Formula IX can be fluorinated to form compounds of Formula XVIII. Compounds of Formula XVIII can be reacted with N,N′-carbonyldiimidazole to form compounds of Formula XIX. Compounds of Formula XIX can be reacted with compounds of Formula R—W—NH₂ to form compounds of Formula XII (wherein W and R are the same as defined earlier). Compounds of Formula XII can be deprotected to form compounds of Formula XIII.

Compounds of Formula IV can be reacted to form compounds of Formula XIV in one or more solvents, for example, dichloromethane, chloroform, carbon tetrachloride, dichloroethane or mixtures thereof. These reactions can also be carried out in the presence of one or more organic bases, for example, triethylamine, pyridine, diisopropylethylamine, 4-(N-dimethylamino)pyridine or mixtures thereof.

Compounds of Formula XIV can be reacted with one or more organic bases in one or more solvents, for example, dimethylformamide, tetrahydrofuran, dimethylsulfoxide or mixtures thereof.

Compounds of Formula XV can be oxidized in the presence of one or more oxidizing agents, for example, Dess-Martin periodinane, N-chlorosuccinimide, pyridinium chlorochromate, Swern Oxidation reagent (oxalyl chloride and dimethylsulfoxide), Pfitzner-Moffatt Oxidation reagent (dicyclohexylcarbodiimide and dimethylsulfoxide), Jones Oxidation reagent (chromic acid, aqueous sulfuric acid and acetone), pyridinium dichromate, 1-ethyl-3-(3-dimethyl aminopropyl)carbodiimide hydrochloride or mixtures thereof. Such oxidation reactions can also be carried out in one or more solvents, for example, chloroform, dichloromethane, carbon tetrachloride, dimethylsulfoxide, dichloroethane or mixtures thereof.

Compounds of Formula XVI can be desmethylated in the presence of one or more desmethylating agents, for example, N-iodo succinimide, iodine in acetic acid, diisopropyl azodicarboxylate or mixtures thereof. The desmethylation reactions can also be carried out in one or more solvents, for example, acetonitrile, tetrahydrofuran, dichloromethane, dichloroethane, chloroform, carbontetrachloride, ethyl acetate or mixtures thereof.

Desmethylation reactions can be quenched in the presence of one or more quenching agents, for example, sodium bisulfite, sodium carbonate, potassium carbonate, cesium carbonate, sodium acetate or mixtures thereof.

Compounds of Formula XVII can be alkylated with one or more reagents of Formula R²CHO, R₂²CO or R²X in one or more solvents, for example, dimethylformamide, acetonitrile, methanol, acetone, tetrahydrofuran or mixtures thereof. Alkylation reactions can also be carried out in the presence of one or more inorganic or organic bases, for example, sodium hydrogen carbonate, potassium carbonate, cesium carbonate, sodium hydride, pyridine, triethylamine, sodium acetate, sodium thiosulfate, diisopropylethylamine or mixtures thereof.

Compounds of Formula XVII can be fluorinated in the presence of one or more fluorinating agents, for example, N-fluorobenzene sulfonimide, selectfluor or mixtures thereof. The fluorination reactions can also be carried out by procedures described in G. Sankar Lai and Syvret R. G., Chem. Rev., 96:1737-1755 (1996). The fluorination reactions can also be carried out in one or more solvents, for example, dimethylformamide, tetrahydrofuran, dimethylsulfoxide or mixtures thereof. The fluorination reactions can also be carried out in the presence of one or more inorganic bases, for example, potassium carbonate, cesium carbonate, sodium hydride, sodium acetate, sodium thiosulfate, potassium tert-butoxide or mixtures thereof.

Compounds of Formula XVIII can be reacted with N,N′-carbonyldiimidazole in one or more solvents, for example, dimethylformamide, acetonitrile, tetrahydrofuran or mixtures thereof. These reactions can also be carried out in the presence of one or more inorganic bases, for example, sodium hydrogen carbonate, potassium carbonate, cesium carbonate, sodium acetate, sodium thiosulfate, sodium hydride or mixtures thereof.

Compounds of Formula XIX can be reacted with compounds of Formula R—W—NH₂ in one or more solvent systems, for example, dimethylformamide/water, acetonitrile/water, dimethylformamide or combinations thereof.

Compounds of Formula XII can be deprorected in one or more alcohols, for example, methanol, ethanol, propanol, isopropanol or mixtures thereof.

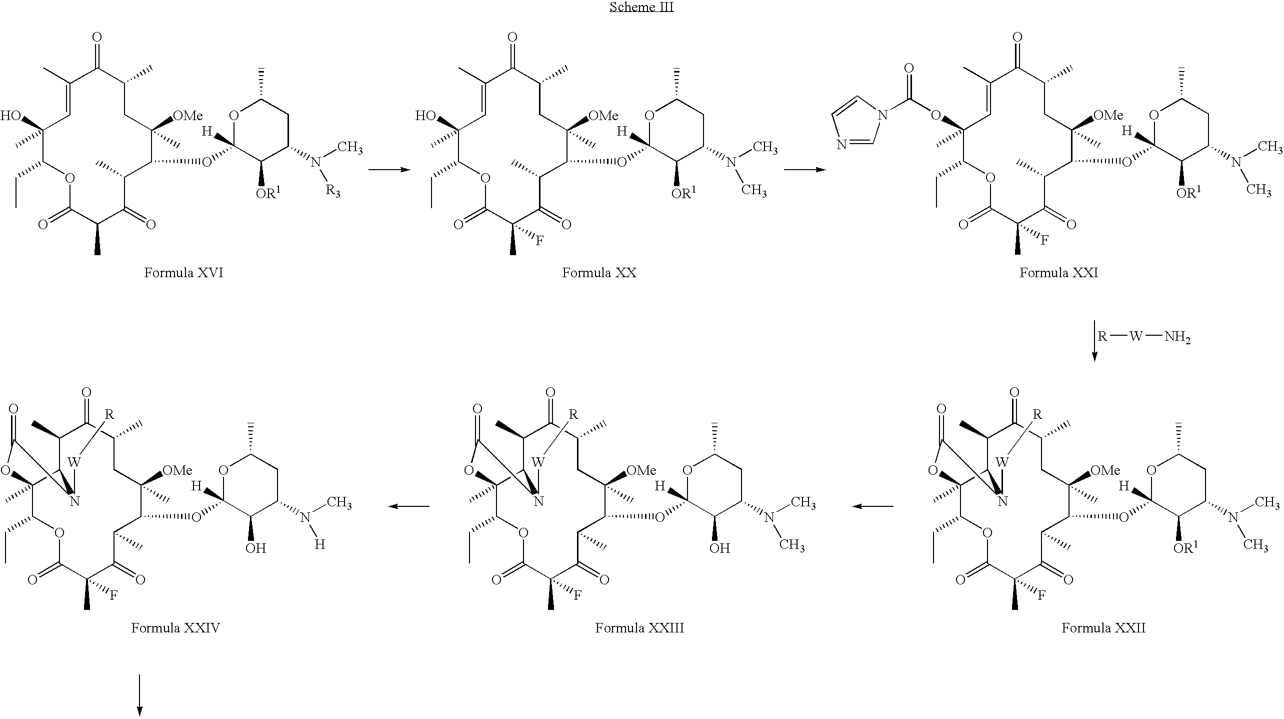

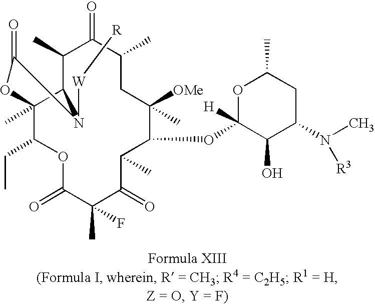

Compounds of Formula XIII can also be prepared according to Scheme III. Thus, Compounds of Formula XVI can be fluorinated to form compounds of Formula XX. Compounds of Formula XX can be reacted with N,N′-carbonyldiimidazole to form compounds of Formula XXI. Compounds of Formula XXI can be reacted with compounds of Formula R—W—NH₂ to form compounds of Formula XXII (wherein R and W are the same as defined earlier). Compounds of Formula XXII can be deprotected to form compounds of Formula XXIII. Compounds of Formula XXIII can be desmethylated at the 3′-N-dimethyl group to form compounds of Formula XXIV. Compounds of Formula XXIV can be alkylated with one or more reagents of Formula R³CHO, R³₂CO or R³X (wherein X is halogen) to form compounds of Formula XIII (wherein R³ is the same as defined earlier).

Compounds of Formula XVI can be fluorinated in the presence of one or more fluorinating agents, for example, N-fluorobenzene sulfonimide, selectfluor or mixtures thereof. The fluorination reactions can also be carried out by procedures described in G. Sankar Lai and Syvret R. G., Chem. Rev., 96:1737-1755 (1996). The fluorination reactions can also be carried out in one or more solvents, for example, dimethylformamide, tetrahydrofuran, dimethyl sulfoxide or mixtures thereof. The fluorination reactions can also be carried out in the presence of one or more inorganic bases, for example, potassium carbonate, sodium hydride, sodium acetate, sodium hydrogen carbonate, cesium carbonate, sodium thiosulfate, potassium tert-butoxide, sodium tert-butoxide, lithium diisopropylamide, sodium methoxide, sodium ethoxide, potassium methoxide, potassium ethoxide, sodium isopropoxide, potassium isopropoxide, lithium carbonate or mixtures thereof.

Compounds of Formula XX can be reacted with N,N′-carbonyldiimidazole in one or more solvents, for example, dimethylformamide, tetrahydrofuran, acetonitrile or mixtures thereof. These reactions can also be carried out in the presence of one or more inorganic bases, for example, sodium hydrogen carbonate, potassium carbonate, cesium carbonate, sodium acetate, sodium thiosulfate, sodium hydride or mixtures thereof.

Compounds of Formula XXI can be reacted with compounds of Formula R—W—NH₂ in one or more solvent systems, for example, acetonitrile/water, dimethylformamide/water or combinations thereof.

Compounds of Formula XXII can be deprotected in one or more alcohols, for example, methanol, ethanol, propanol, isopropanol or mixtures thereof.

Compounds of Formula XXIII can be desmethylated in one or more solvents, for example, acetonitrile, tetrahydrofuran, dichloromethane, dichloroethane, chloroform, carbontetrachloride, ethyl acetate or mixtures thereof. These desmethylation reactions can also be carried out in the presence of one or more desmethylating agents, for example, N-iodosuccinimide, iodine in acetic acid, diisopropyl azodicarboxylate or mixtures thereof.

The desmethylation reactions can be quenched in the presence of one or more quenching agents, for example, sodium bisulfite, sodium carbonate, potassium carbonate, cesium carbonate, sodium acetate or mixtures thereof.

Compounds of Formula XXIV can be alkylated with one or more reagents of Formula R³CHO, R³₂CO or R³X in one or more solvents, for example, dimethylformamide, acetonitrile, methanol, acetone, tetrahydrofuran or mixtures thereof. These alkylation reactions can be carried out in the presence of oen or more inorganic or organic bases, for example, sodium hydrogen carbonate, potassium carbonate, cesium carbonate, sodium hydride, pyridine, triethylamine, sodium acetate, sodium thiosulfate, diisopropyl ethylamine or mixtures thereof.

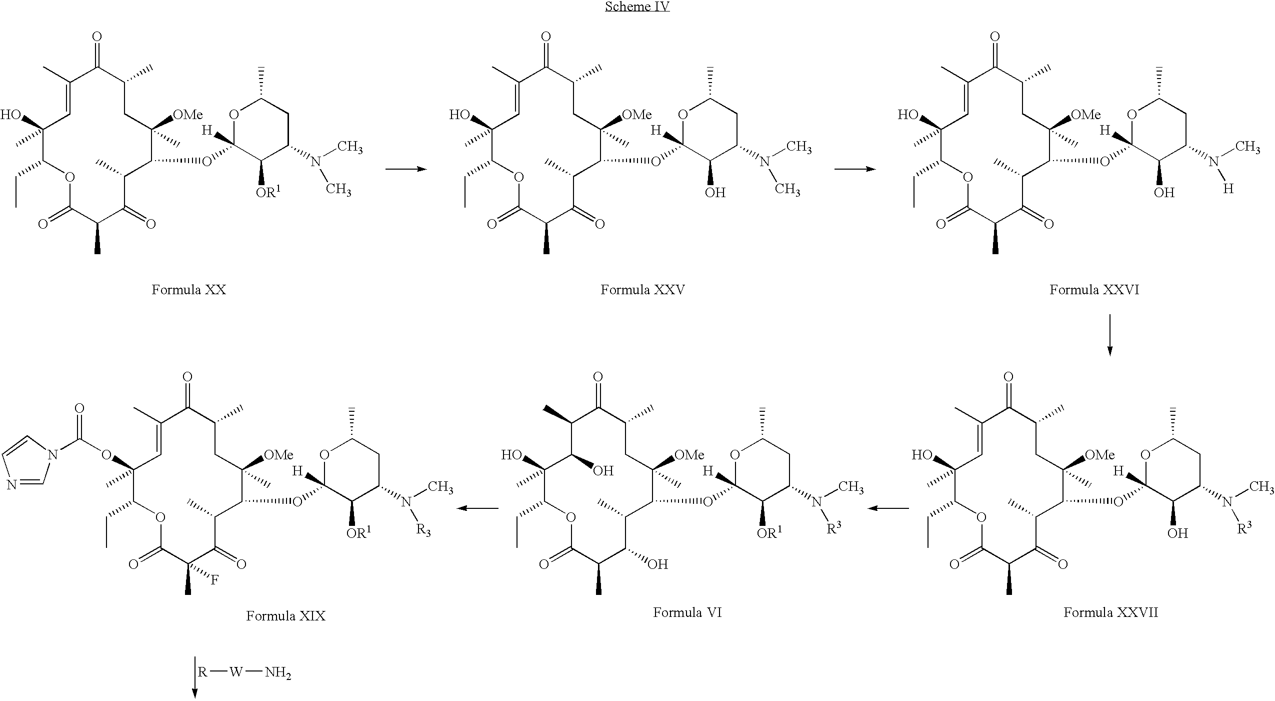

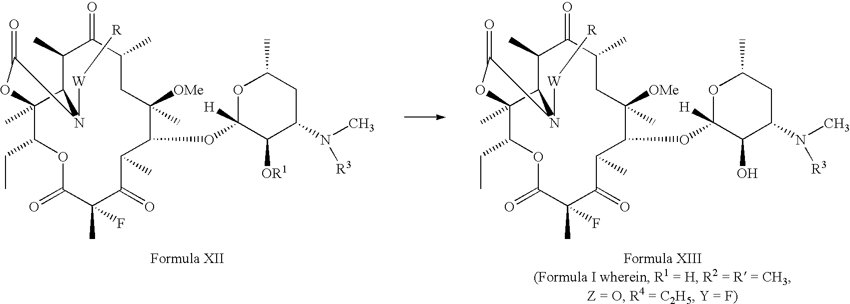

Compounds of Formula XIII can also be prepared according to Scheme IV. Thus, compounds of Formula XX (wherein R¹ is COPh) can be deprotected to form compounds of Formula XXV. Compounds of Formula XXV can be desmethylated at the 3′-N-dimethyl group to form compounds of Formula XXVI. Compounds of Formula XXVI can be alkylated with one or more reagents of Formula R³CHO, R³₂CHO or R³X (X is halogen) to form compounds of Formula XXVII (R³ is the same as defined earlier). Compounds of Formula XXVII can be protected with one or more reagents of Formula R¹₂O or R¹X (wherein X is halogen) to form compounds of Formula XVIII (wherein R¹ is COCH₃). Compounds of Formula XVIII can be reacted with N,N′-carbonyldiimidazole to form compounds of Formula XIX. Compounds of Formula XIX can be reacted with compounds of Formula R—W—NH₂ to form compounds of Formula XII (R is the same as defined earlier). Compounds of Formula XII can be deprotected to form compounds of Formula XIII.

Compounds of Formula XX can be deprotected in one or more alcohols, for example, methanol, ethanol, propanol, isopropanol or mixtures thereof.

Compounds of Formula XXV can be desmethylated in the presence of one or more desmethylating agents, for example, N-iodosuccinimide, iodine in acetic acid, diisopropyl azodicarboxylate or mixtures thereof. Desmethylation reactions can also be carried out in one or more solvents, for example, acetonitrile, tetrahydrofuran, dichloromethane, dichloroethane, chloroform, carbon tetrachloride, ethyl acetate or mixtures thereof.

The desmethylation reactions can be quenched in the presence of one or more quenching agents, for example, sodium bisulfite, sodium carbonate, potassium carbonate, cesium carbonate, sodium acetate or mixtures thereof.

Compounds of Formula XXVI can be alkylated by reaction with one or more reagents of Formula R³CHO, R³₂CO on R³X in one or more solvents, for example, dimethylformamide, acetonitrile, tetrahydrofuran, acetone, methanol or mixtures thereof. Alkylation reactions can also be carried out in the presence of one or more inorganic or organic bases, for example, sodium hydrogen carbonate, potassium carbonate, cesium carbonate, sodium hydride, pyridine, triethylamine, sodium acetate, sodium thiosulfate, diisopropyl ethylamine or mixtures thereof.

Compounds of Formula XXVII can be hydroxyl protected by reaction with one or more reagents of Formula R¹₂O or R¹X in one or more solvents, for example, dichloromethane, dichloroethane, carbontetrachloride, chloroform, acetone or mixtures thereof. Hydroxyl protection reactions can also be carried out in the presence of one or more inorganic bases, for example, potassium carbonate, cesium carbonate, sodium carbonate, sodium bicarbonate, potassium bicarbonate or mixtures thereof.

Compounds of Formula XVIII can be reacted with N,N′-carbonyldiimidazole in one or more solvents, for example, dimethylformamide, tetrahydrofuran, acetonitrile or mixtures thereof. Such reactions can also be carried out in the presence of one or more inorganic bases, for example, sodium hydrogen carbonate, potassium carbonate, cesium carbonate, sodium acetate, sodium thiosulfate, sodium hydride or mixtures thereof.

Compounds of Formula XIX can be reacted with compounds of Formula R—W—NH₂ in one or more solvent systems, for example, acetonitrile/water, dimethylformamide/water, dimethyformamide or combinations thereof.

Compounds of Formula XII can be deprotected in one or more alcohols, for example, methanol, ethanol, propanol, isopropanol or mixtures thereof.

In the above schemes, where specific reagents, for example, bases, acids oxidizing agents, solvents, etc., are described, it is to be understood that other reagents, e.g., bases, acids, oxidizing agents, solvents, etc., known to one of ordinary skill in the art may be used. Similarly, reaction temperatures and durations may be adjusted according to the desired needs without undue experimentation and well within the abilities of one of ordinary skill in the art. All the epimers, unless otherwise specified in the above schemes, are also encompassed within the scope of the invention.

Compounds of the present invention useful for such purpose are listed below:
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-imidazol-1-yl)-propyl)-imino]erythromycin A (Compound No. 1),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((1H)-imidazo(4,5-b)-pyridin-1-yl)-butyl)-imino)]erythromycin A (Compound No. 2),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((3H)-imidazo(4,5-b)-pyridin-1-yl)-butyl)-imino)]erythromycin A (Compound No. 3),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(benzoimidazol-1-yl)-butyl)-imino)]erythromycin A (Compound No. 4),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(imidazol-1-yl)-butyl)-imino)]erythromycin A (Compound No. 5),
2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((3-imidazol-1-yl)-propyl)-imino]erythromycin A (Compound No. 6),
2-α-Fluoro-5-O-(3′-N-desmethyl-3′-N-isopropyl)-11,12-dideoxy-3-O-decladinosyl-6-O-methyl-3-oxo-12,11-[oxycarbonyl-((3-imidazol-1-yl)-propyl)-imino]erythromycin A (Compound No. 7),
2-α-Fluoro-5-O-(3′-N-desmethyl-3′-N-n-propyl)-11,12-dideoxy-3-O-decladinosyl-6-O-methyl-3-oxo-12,11-[oxycarbonyl-((3-imidazol-1-yl)-propyl)-imino]erythromycin A (Compound No. 8),
2-α-Fluoro-5-O-(3′-N-desmethyl-3′-N-n-propyl)-11,12-dideoxy-3-O-decladinosyl-6-O-methyl-3-oxo-12,11-[oxycarbonyl-((4-(benzoimidazol-1-yl)-butyl)-imino)]erythromycin A (Compound No. 9),
2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-(pyrrolo-(2,3-b)pyridin-1-yl)-butyl)-imino)]erythromycin A (Compound No. 10),
2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-(pyrrolo-(2,3-b)pyridin-1-yl)-butyl)-imino)]erythromycin A (Compound No. 11),
2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-(benzoimidazol-1-yl)-butyl)-imino)]erythromycin A (Compound No. 12),
2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-(benzoimidazol-1-yl)-butyl)-imino)]erythromycin A (Compound No. 13),
2-α-Fluoro-5-O-(3′-N-desmethyl-3′-N-n-propyl)-11,12-dideoxy-3-O-decladinosyl-6-O-methyl-3-oxo-12,11-[oxycarbonyl-((4-(imidazol-1-yl)-butyl)-imino)]erythromycin A (Compound No. 14),
2-α-Fluoro-5-O-(3′-N-desmethyl-3′-N-ethyl)-11,12-dideoxy methyl-3-oxo-12,11-[oxycarbonyl-((4-(pyrrolo-(2,3-b)-pyridin-1-yl)-butyl)-imino)]erythromycin A (Compound No. 15),
2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-((1H)-imidazo(4,5-b)-pyridin-1-yl)-butyl)-imino)]erythromycin A (Compound No. 16),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(purin-9-yl)-butyl)-imino)]erythromycin A (Compound No. 17),
2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-((3H)-imidazo(4,5-b)-pyridin-1-yl)-butyl)-imino)]erythromycin A (Compound No. 18),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-pyridin-3-yl-imidazol-1-yl)-butyl)-imino)]erythromycin A (Compound No. 19),
2-α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-quinolin-4-ylmethyl)-2-aminoethyl)-imino]erythromycin A (Compound No. 20),
2-α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-pyridine-3-ylmethyl)-2-aminoethyl)-imino]erythromycin A (Compound No. 21),
2-α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N-pyridine-4-ylmethyl)-2-aminoethyl)-imino]erythromycin A (Compound No. 22),
2-α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-pyridine-2-ylmethyl)-2-aminoethyl)-imino]erythromycin A (Compound No. 23),
2-α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-pyridine-2-ylmethyl)-2-aminoethyl)-imino]erythromycin A (Compound No. 24),
2-α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-pyridine-3-ylmethyl)-2-aminoethyl)-imino]erythromycin A (Compound No. 25),
2-α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-pyridine-4-ylmethyl)-2-aminoethyl)-imino]erythromycin A (Compound No. 26),
2-α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-quinoline-4-ylmethyl)2-aminoethyl)-imino]erythromycin A (Compound No. 27),
2-α-Fluoro-5-O-(3′-N-desmethyl-3′-N-ethyl)-11,12-dideoxy-3-O-decladinosyl-6-O-methyl-3-oxo-12,11-[oxycarbonyl-((N′-pyridine-3-ylmethyl)-2-aminoethyl)-imino]erythromycin A (Compound No. 28),
2-α-Fluoro-5-O-(3′-N-desmethyl-3′-N-allyl)-11,12-dideoxy-3-O-decladinosyl-6-O-methyl-3-oxo-12,11-[oxycarbonyl-((N′-acetyl-N′-pyridine-3-ylmethyl)-2-aminoethyl)-imino]erythromycin A (Compound No. 29),
2-α-Fluoro-5-O-(3′-N-desmethyl-3′-N-allyl)-11,12-dideoxy-3-O-decladinosyl-6-O-methyl-3-oxo-12,11-[oxycarbonyl-((N′-pyridine-3-ylmethyl)-2-aminoethyl)-imino]erythromycin A (Compound No. 30),
2-α-Fluoro-5-O-(3′-N-desmethyl-3′-N-ethyl)-11,12-dideoxy-3-O-decladinosyl-6-O-methyl-3-oxo-12,11-[oxycarbonyl-((N′-acetyl-N′-pyridine-3-ylmethyl)-2-aminoethyl)-imino]erythromycin A (Compound No. 31),
2-α-Fluoro-5-O-(3′-N-desmethyl-3′-N-ethyl)-11,12-dideoxy-3-O-decladinosyl-6-O-methyl-3-oxo-12,11-[oxycarbonyl-((N′,N′-di-pyridine-3-ylmethyl)-2-aminoethyl)-imino]erythromycin A (Compound No. 32),
2-α-Fluoro-5-O-(3′-N-desmethyl-3′-N-allyl)-11,12-dideoxy-3-O-decladinosyl-6-O-methyl-3-oxo-12,11-[oxycarbonyl-((N′,N′-di-pyridine-3-ylmethyl)-2-aminoethyl)-imino]erythromycin A (Compound No. 33),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-(quinolin-3-yl)-butyl)-imino)]erythromycin A (Compound No. 34),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(quinolin-8-yl)-butyl)-imino)]erythromycin A (Compound No. 35),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-2-yl)-butyl)-imino)]erythromycin A (Compound No. 36),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-3-yl)-butyl)-imino)]erythromycin A (Compound No. 37),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-4-yl)-butyl)-imino)]erythromycin A (Compound No. 38),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-(pyridin-2-yl)-butyl)-imino)]erythromycin A (Compound No. 39),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-(pyridin-4-yl)-butyl)-imino)]erythromycin A (Compound No. 40),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-(pyridin-3-yl)-pentyl)-imino]erythromycin A (Compound No. 41),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-(pyridin-3-yl)-butyl)-imino)]erythromycin A (Compound No. 42),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(indol-1-yl)-butyl)-imino)]erythromycin A (Compound No. 43),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A (Compound No. 44),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((5-nitro)-indol-1-yl)-butyl)-imino)]erythromycin A (Compound No. 45),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((1H)-imidazo[4,5-c]pyridin-1-yl)-butyl)-imino)]erythromycin A (Compound No. 46),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(benzotriazol-2-yl)-butyl)-imino)]erythromycin A (Compound No. 47),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((3H)-imidazo[4,5-c]pyridin-3-yl)-butyl)-imino)]erythromycin A (Compound No. 48),
2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A (Compound No. 49),
2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-(benzotriazol-1-yl)-butyl)-imino)]erythromycin A (Compound No. 50),
2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-(benzotriazol-2-yl)-butyl)-imino)]erythromycin A (Compound No. 51),
2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-((1H)-imdazo[4,5-b]pyridin-1-yl)-butyl)-imino)]erythromycin A (Compound No. 52),
2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-(indol-1-yl)-butyl)-imino)]erythromycin A (Compound No. 53),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-(benzotriazol-1-yl)-butyl)-imino)]erythromycin A (Compound No. 54),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-(benzoimidazol-1-yl)-butyl)-imino)]erythromycin A (Compound No. 55),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A (Compound No. 56),
2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A (Compound No. 57),
2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-(benzotriazol-2-yl)-butyl)-imino)]erythromycin A (Compound No. 58),
2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-(indol-1-yl)-butyl)-imino)]erythromycin A (Compound No. 59),
2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A (Compound No. 60),
2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-((5-nitro)-indol-1-yl)-butyl)-imino)]erythromycin A (Compound No. 61),
2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-((5-nitro)-indol-1-yl)-butyl)-imino)]erythromycin A (Compound No. 62),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((5-fluoro)-indol-1-yl)-butyl)-imino)]erythromycin A (Compound No. 63),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(benzotriazol-1-yl)-butyl)-imino)]erythromycin A (Compound No. 64),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-methyl)-benzoimidazol-1-yl)-butyl)-imino)]erythromycin A (Compound No. 65),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-ethyl)-benzoimidazol-1-yl)-butyl)-imino)]erythromycin A (Compound No. 66),
2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-(benzotriazol-1-yl)-butyl)-imino)]erythromycin A (Compound No. 67),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((2-ethyl)-benzoimidazol-1-yl)-butyl)-imino)]erythromycin A (Compound No. 68),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-pyridin-3-yl-imidazol)-1-yl)-butyl)-imino)]erythromycin A (Compound No. 69),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((2-methyl)-benzoimidazol-1-yl)-butyl)-imino)]erythromycin A (Compound No. 70),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A (Compound No. 71),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((1H)-imidazo[4,5-b]pyridin-1-yl)-butyl)-imino)]erythromycin A (Compound No. 72),
2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A (Compound No. 73),
2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((5-nitro)-indol-1-yl)-butyl)-imino)]erythromycin A (Compound No. 74),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((2-pyridin-3-ylmethyl)-benzoimidazol-1-yl)-butyl)-imino)]erythromycin A (Compound No. 75),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-pyridin-3-ylmethyl)-benzoimidazol-1-yl)-butyl)-imino)]erythromycin A (Compound No. 76),
2-α-Fluoro-5-O-(3*-N-desmethyl-3′-N-allyl)-11,12-dideoxy-3-O-decladinosyl-6-O-methyl-3-oxo-12,11-[oxycarbonyl-((4-((2-phenyl)-imidazol-1-yl)-butyl)-imino)]erythromycin A (Compound No. 77),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((5-fluoro)-indol-1-yl)-butyl)-imino)]erythromycin A (Compound No. 78),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((5,6-dimethyl)-benzoimidazol-1-yl)-butyl)-imino)]erythromycin A (Compound No. 79),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((5,6-dimethyl)-benzoimidazol-1-yl)-butyl)-imino)]erythromycin A (Compound No. 80),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((3H)-imidazo[4,5-c]pyridin-3-yl)-butyl)-imino)]erythromycin A (Compound No. 81),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((2-methyl)-(3H)-imidazo[4,5-b]pyridin-3-yl)-butyl)-imino)]erythromycin A (Compound No. 82),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-methyl)-(3H)-imidazo[4,5-b]pyridin-3-yl)-butyl)-imino)]erythromycin A (Compound No. 83),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((1H)-imidazo[4,5-c]pyridin-1-yl)-butyl)-imino)]erythromycin A (Compound No. 84),
2-α-Fluoro-5-O-(3′-N-desmethyl-3′-N-ethyl)-11,12-dideoxy-3-O-decladinosyl-6-O-methyl-3-oxo-12,11-[oxycarbonyl-((9-(4-amino-butyl)-9H-purin-6-yl)-imino]erythromycin A (Compound No. 85),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-phenyl)-imidazol-1-yl)-butyl)-imino)]erythromycin A (Compound No. 86),
2-α-Fluoro-5-O-(3′-N-desmethyl-3′-N-acetyl)-11,12-dideoxy-3-O-decladinosyl-6-O-methyl-3-oxo-12,11-[oxycarbonyl-((4-(benzoimidazol-1-yl)-butyl)-imino)]erythromycin A (Compound No. 87),
2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A (Compound No. 88),
2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-(benzoimidazol-1-yl)-butyl)-imino)]erythromycin A (Compound No. 89),
2-α-Fluoro-5-O-(3′-N-desmethyl-3′-N-cyclopropylmethyl)-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A (Compound No. 90),
2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A (Compound No. 91),
2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-(benzotriazol-1-yl)-butyl)-imino)]erythromycin A (Compound No. 92),
2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-(benzoimidazol-1-yl)-butyl)-imino)]erythromycin A (Compound No. 93),
2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A (Compound No. 94),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-trifluoromethyl)-benzoimidazol-1-yl)-butyl)-imino)]erythromycin A (Compound No. 95),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4,5-diphenyl)-imidazol-1-yl)-butyl)-imino)]erythromycin A (Compound No. 96),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4,5-diphenyl)-imidazol-1-yl)-butyl)-imino)]erythromycin A (Compound No. 97),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A (Compound No. 98),
2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A (Compound No. 99),
2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-(benzotriazol-1-yl)-butyl)-imino)]erythromycin A (Compound No. 100),
2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((4-pyridin-3-yl)-imidazol-1-yl)-butyl)-imino)]erythromycin A (Compound No. 101),
2-α-Fluoro-5-O-(3′-N-desmethyl-3′-N-acetyl)-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A (Compound No. 102),
2-α-Fluoro-5-O-(3′-N-desmethyl-3′-N-acetyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A (Compound No. 103),
2-α-Fluoro-5-O-(3′-N-desmethyl-3′-N-acetyl)-11,12-dideoxy-3-O-decladinosyl-6-O-methyl-3-oxo-12,11-[oxycarbonyl-((4-((1H)-imidazo[4,5-b]pyridin-1-yl)-butyl)-imino)]erythromycin A (Compound No. 104),
2-α-Fluoro-5-O-(3′-N-desmethyl-3′-N-acetyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A (Compound No. 105),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((3,5-diphenyl)-pyrazol-1-yl)-butyl)-imino)]erythromycin A (Compound No. 106),
2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((4-pyridin-3-yl)-imidazol-1-yl)-butyl)-imino)]erythromycin A (Compound No. 107),
2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((1H)-imidazo[4,5-b]pyridin-1-yl)-butyl)-imino)]erythromycin A (Compound No. 108),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((3,5-diphenyl)-pyrazol-1-yl)-butyl)-imino)]erythromycin A (Compound No. 109),
2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((1H)-imidazo[4,5-b]pyridin-1-yl)-butyl)-imino)]erythromycin A (Compound No. 110),
2-α-Fluoro-5-O-(3′-N-desmethyl-3′-N-acetyl)-11,12-dideoxy-3-O-decladinosyl-6-O-methyl-3-oxo-12,11-[oxycarbonyl-((4-((4-pyridin-3-yl)-imidazo)-1-yl)-butyl)-imino)]erythromycin A (Compound No. 111),
2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((4-(2,4-difluoro)-phenyl)-imidazol-1-yl)-butyl)-imino)]erythromycin A (Compound No. 112),
2-α-Fluoro-5-O-(3′-N-desmethyl-3′-N-2-ethyl)-11,12-dideoxy-3-O-decladinosyl-6-O-methyl-3-oxo-12,11-[oxycarbonyl-((4-((4-(2,4-difluoro)-phenyl)-imidazol)-1-yl)-butyl)-imino)]erythromycin A (Compound No. 113),
2-α-Fluoro-5-O-(3′-N-desmethyl-3′-N-2-allyl)-11,12-dideoxy-3-O-decladinosyl-6-O-methyl-3-oxo-12,11-[oxycarbonyl-((4-((4-(2,4-difluoro)-phenyl)-imidazol)-1-yl)-butyl)-imino)]erythromycin A (Compound No. 114),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-furan-3-yl)-imidazol-1-yl)-butyl)-imino)]erythromycin A (Compound No. 115),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-furan-3-yl)-imidazol-1-yl)-butyl)-imino)]erythromycin A hydrochloride salt (Compound No. 115a),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-phenyl-pyrazol-1-yl)-butyl)-imino))]erythromycin A (Compound No. 116),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-pyridin-3-yl-1H-imidazol-1-yl)-pentyl)-imino)]erythromycin A (Compound No. 117),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-pyridin-4-yl-1H-imidazol-1-yl)-butyl)-imino))]erythromycin A (Compound No. 118)
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-pyridin-4-yl-1H-imidazol-1-yl)-butyl)-imino))]erythromycin A (Compound No. 119),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-phenyl-pyrazol-1-yl)-butyl)-imino)) erythromycin A (Compound No. 120),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((indazol-2-yl)-butyl)-imino))]erythromycin A (Compound No. 121),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((indazol-2-yl)-butyl)-imino))]erythromycin (Compound No. 122),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((3-pyridin-3-yl-pyrazol-1-yl)-butyl)-imino))]erythromycin A (Compound No. 123),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((indazol-1-yl)-butyl)-imino))]erythromycin A (Compound No. 124),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-pyrazin-2-yl-1H-imidazol-1-yl)-butyl)-imino))]erythromycin A (Compound No. 125),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-thiophen-3-yl-1H-imidazol-1-yl)-butyl)-imino))]erythromycin A (Compound No. 126),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-thiophen-3-yl-1H-imidazol-1-yl)-butyl)-imino))]erythromycin A (Compound No. 127),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-quinolin-3-yl-1H-imidazol-1-yl)-butyl)-imino))]erythromycin A (Compound No. 128),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-(indazol-1-yl)-butyl)-imino))]erythromycin A (Compound No. 129),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-pyrimidin-5-yl-1H-imidazol-1-yl)-butyl)-imino))]erythromycin A (Compound No. 130),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-pyrimidin-5-yl-1H-imidazol-1-yl)-butyl)-imino))]erythromycin A (Compound No. 131),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-furan-2-yl-1H-imidazol-1-yl)-butyl)-imino))]erythromycin A (Compound No. 132),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-thiophen-2-yl-1H-imidazol-1-yl)-butyl)-imino))]erythromycin A (Compound No. 133),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(4-chloro-phenyl)-1H-imidazol-1-yl)-butyl)-imino))]erythromycin A (Compound No. 134),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-(4-chloro-phenyl)-1H-imidazol-1-yl)-butyl)-imino))]erythromycin A (Compound No. 135),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-pyridin-2-yl-1H-imidazol-1-yl)-butyl)-imino))]erythromycin A (Compound No. 136),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-pyridin-2-yl-1H-imidazol-1-yl)-butyl)-imino))]erythromycin A (Compound No. 137),
2-α-Fluoro-5-O-(3′-N-desmethyl-3′-N-allyl)-11,12-dideoxy-3-O-decladinosyl-6-O-methyl-3-oxo-12,11-[oxycarbonyl-((9-(4-amino-butyl)-9H-purin-6-yl)-imino)]erythromycin A (Compound No. 138),
2-α-Fluoro-5-O-(3′-N-desmethyl-3′-N-allyl)-11,12-dideoxy-3-O-decladinosyl-6-O-methyl-3-oxo-12,11-[oxycarbonyl-((4-((3-pyridin-3-yl-1H-pyrazol-1-yl)-butyl)-imino))]erythromycin A (Compound No. 139),
2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((4-(4-chloro-phenyl)-1H-imidazol-1-yl)-butyl)-imino))]erythromycin A (Compound No. 140),
2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((4-pyridin-2-yl)-1H-imidazol-1-yl)-butyl)-imino))]erythromycin A (Compound No. 141),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-Imidazo[4,5-b]pyridine-2-yl)-butyl)-imino)erythromycin A (Compound No. 142),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Tetrazol-1-yl-imidazol-1-yl)-butyl)-imino)erythromycin A (Compound No. 143),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(1H-Benzoimidazol-2-yl)-butyl)-imino)erythromycin A (Compound No. 144),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[4-(6-Fluoro-pyridin-3-yl)-imidazol-1-yl]-butyl)-imino)erythromycin A (Compound No. 145),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3-H-Imidazo[4,5-c]pyridine-2-yl)-butyl)iminoerythromycin A (Compound No. 146),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-[1,2,4]Trizol-1-yl-phenyl)-butyl)-imino)erythromycin A (Compound No. 147),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Imidazol-1-yl-phenyl)-butyl)-imino)erythromycin A (Compound No. 148),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[4-(6-Chloro-pyridin-3-yl)-imidazol-1-yl]-butyl)-imino)erythromycin A (Compound No. 149),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(3-[1-(4-Amino-butyl)-1H-imidazol-4-yl]-phenyl)iminoerythromycin A (Compound No. 150),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Pyridin-3-yl-thiazol-2-yl)-butyl)-imino)erythromycin A (Compound No. 151),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(3-[2-(4-Amino-butyl)-thiazol-4-yl]-phenyl)iminoerythromycin A (Compound No. 152),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Pyrazol-1-yl-imidazol-1-yl)-butyl)-imino)erythromycin A (Compound No. 153),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-([1,4′]-Bipyrazolyl-1′-yl)-butyl)-imino)erythromycin A (Compound No. 154),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Imidazol-1-yl-pyrazol-1-yl)-butyl)iminoerythromycin A (Compound No. 155),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Pyrazol-1-yl-imidazol-1-yl)-butyl)iminoerythromycin A (Compound No. 156),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[3,3′]Bithiophenyl-5-yl-butyl)-imino)erythromycin A (Compound No. 157),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[2,3′]Bithiophenyl-5′-yl-butyl)-imino)erythromycin A (Compound No. 158),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Furan-2-yl-thiaophen-2-yl)-butyl)-imino)erythromycin A (Compound No. 159),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Oxazol-5-yl-imidazol-1-yl)-butyl)-imino)erythromycin A (Compound No. 160),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3-Pyrrol-1-yl-[1,2,4]triazol-1-yl)-butyl)iminoerythromycin A (Compound No. 161),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-Thiophen-2-yl-tetrazol-2-yl)-butyl)-imino)erythromycin A (Compound No. 162),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Thiophen-3-yl-pyrazol-1-yl)-butyl)-imino) erythromycin A (Compound No. 163),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Furan-3-yl-pyrazol-1-yl)-butyl)-imino)erythromycin A (Compound No. 164),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Furan-2-yl-pyrazol-1-yl)-butyl)-imino)erythromycin A (Compound No. 165),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-Phenyl-tetrazol-2-yl)-butyl)-imino)erythromycin A (Compound No. 166),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[5-(4-Methoxy-phenyl)-tetrazol-2-yl]-butyl)-imino)erythromycin A (Compound No. 167),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-Furan-3-yl-imidazol-1-yl)-butyl)-imino)erythromycin A (Compound No. 168),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-Pyrazol-1-yl-pyridin-3-yl)-butyl)-imino)erythromycin A (Compound No. 169),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Pyridin-3-yl-pyrazol-1-yl)-butyl)-imino)erythromycin A (Compound No. 170),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-Thiophen-2-yl-pyridin-3-yl)-butyl)-imino)-erythromycin A (Compound No. 171),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Phenyl-thiophen-2-yl)-butyl)-imino)erythromycin A (Compound No. 172),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(N-(4-Amino-butyl)-N-thiazol-2-yl-nicotinimido)]erythromycin A (Compound No. 173),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(N-(4-Amino-butyl)-N-thiazol-2-yl-nicotinimido)]erythromycin A (Compound No. 174),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[4-(6-Pyrrol-1-yl-pyridin-3-yl)-imidazol-1-yl]-butyl)-imino)erythromycin A (Compound No. 175),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-Pyrrol-1-yl-pyridin-3-yl)-butyl)-imino)erythromycin A (Compound No. 176),
2-∝-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-Pyrrol-1-yl-thiazol-5-yl)-butyl)-imino)erythromycin A (Compound No. 177),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-Imidazol-1-yl-pyridin-3-yl)-butyl)iminoerythromycin A (Compound No. 178),
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[4-(Tetrahydro-furan-2-yl)-pyridin-3-yl]-butyl)iminoerythromycin A (Compound No. 179),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-p-tolyl-imidazol-1-yl)-butyl)-imino)]erythromycin A (Compound No. 180),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-aminophenyl)-imidazol-1-yl)-butyl)-imino)]erythromycin A (Compound No. 181),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-methyl-3-aminophenyl)-imidazol-1-yl)-butyl)-imino)]erythromycin A (Compound No. 182),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(3-imidazol-1-yl)-phenyl)imidazol-1-yl)-butyl)-imino)]erythromycin A (Compound No. 183),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N,N-dicyclopropyl-amino)-phenyl)-imidazol-1-yl)-butyl)-imino)]erythromycin A (Compound No. 184),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N,N-dimethyl-amino)phenyl)-imidazol-1-yl)-butyl)-imino)]erythromycin A (Compound No. 185),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(3-(tetrazol-1-yl)-phenyl)-imidazol-1-yl)-butyl)-imino)]erythromycin A (Compound No. 186),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(2-(pyrrol-1-yl)-thiozol-5-yl)-imidazol-1-yl)-butyl)-imino)]erythromycin A (Compound No. 187),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(3-trifluoromethyl-phenyl)-imidazol-1-yl)-butyl)-imino)]erythromycin A (Compound No. 188),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((N-(thiazol-2-yl)-benzimido)-butyl)-imino)]erythromycin A (Compound No. 189),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(2-amino-pyrimidin)-5-yl)-imidazol-1-yl)-butyl)-imino)]erythromycin A (Compound No. 190),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(4-(pyrrol-1-yl)-phenyl)-imidazol-1-yl)-butyl)-imino)]erythromycin A (Compound No. 191),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(3-(pyrrol-1-yl)-phenyl)-imidazol-1-yl)-butyl)-imino)]erythromycin A (Compound No. 192),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-acetyl-amino)-phenyl)-imidazol-1-yl)-butyl)-imino)]erythromycin A (Compound No. 193),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-isopropyl-amino)-phenyl)-imidazol-1-yl)-butyl)-imino)]erythromycin A (Compound No. 194),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(3-nitrophenyl)-imidazol-1-yl)-butyl)-imino)]erythromycin A (Compound No. 195),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-benzoyl-amino)-phenyl)-imidazol-1-yl)-butyl)-imino)]erythromycin A (Compound No. 196),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(3,4-dimethyl-phenyl)-imidazol-1-yl)-butyl)-imino)]erythromycin A (Compound No. 197),
5-O-(3′-N-desmethyl-3′-N-ethyl)-11,12-dideoxy-3-O-decladinosyl-6-O-methyl-3-oxo-12,11-[oxycarbonyl-(((4-(4-Pyridin-3-yl-thiazol-2-yl)-butyl)-imino)]erythromycin A (Compound No. 198),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(4-fluorophenyl)-imidazol-1-yl)-butyl)-imino)]erythromycin A (Compound No. 199),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(4-methoxy-phenyl)-imidazol-1-yl)-butyl)-imino)]erythromycin A (Compound No. 200),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((N-(benzthiazol-2-yl)-benzimido)-butyl)-imino)]erythromycin A (Compound No. 201),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((N-(thiazol-2-yl)-nicotinamido)-butyl)-imino)]erythromycin A (Compound No. 202),
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-methyl-amino)-phenyl)-imidazol-1-yl)-butyl)-imino)]erythromycin A (Compound No. 203),
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(4-(2-aminopyridyl)-imidazol-1-yl)-butyl)-imino)]erythromycin A (Compound No. 204),
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(5-(2-aminopyridyl)-2H-tetrazol-5-yl)-butyl)-imino)]erythromycin A (Compound No. 205),
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3,4-difluorophenyl)-imidazol-1-yl)-butyl)-imino)]erythromycin A (Compound No. 206),
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(4-(2-chloropyridyl)-imidazol-1-yl)-butyl)-imino)]erythromycin A (Compound No. 207),
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-(6-aminopyridyl)-imidazol-1-yl)-butyl)-imino)]erythromycin A (Compound No. 208),
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(5-(2-Chloro-pyridin-4-yl)-tetrazol-2-yl])-butyl)-imino)]erythromycin A (Compound No. 209),
   pharmaceutically acceptable salts, pharmaceutically acceptable solvates, stereoisomers, prodrugs, metabolites or polymorphs thereof.

The compounds described herein are pharmacologically active against Gram-positive, Gram-negative and anaerobic bacteria and accordingly, are useful as antibacterial agents for treating bacterial infections in a patient in need thereof, for example, in a human or an animal. Because of their antibacterial activity, the compounds described herein may be administered to an animal for treatment orally, topically, rectally, internasally, or by parenteral route. Pharmaceutical compositions described herein comprise pharmaceutically effective amounts of compounds described herein formulated together with one or more pharmaceutically acceptable carriers, excipients or diluents.

Solid form preparations for oral administration include capsules, tablet, pills, powder, granules, cachets and suppositories. For solid form preparations, active compounds can be mixed with one or more inert, pharmaceutically acceptable excipients or carrier, for example, sodium citrate, dicalcium phosphate and/or fillers or extenders (for example, starches, lactose, sucrose, glucose, mannitol, silicic acid or mixtures thereof); binders, for example, carboxymethylcellulose, alginates, gelatins, polyvinylpyrrolidinone, sucrose, acacia or mixtures thereof; disintegrating agents, for example, agar-agar, calcium carbonate, potato starch, alginic acid, certain silicates, sodium carbonate or mixtures thereof; absorption acceletors, for example, quaternary ammonium compounds; wetting agents, for example, cetyl alcohol, glycerol mono stearate or mixtures thereof; adsorbents, for example, Kaolin; lubricants, for example, talc, calcium stearate, magnesium stearate, solid polyethyleneglycol, sodium luaryl sulfate or mixtures thereof; or mixtures thereof.

Capsules, tablets or pills may also comprise buffering agents.

Tablets, capsules, pills or granules can be prepared using one or more coatings or shells, for example, enteric coatings or other coatings known to one of ordinary skill in the art.

Liquid form preparations for oral administration include pharmaceutically acceptable emulsions, solutions, suspensions, syrups or elixirs. In such liquid form preparations, active compounds can be mixed with water or one or more other solvents, solubilizing agents or emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, for example, cottonseed, groundnut, corn, germ, olive, castor and sesame oil), glycerol, fatty acid esters of sorbitan or mixtures thereof. Oral compositions can also include one or more adjuants, for example, wetting agents, emulsifying agents, suspending agents, sweetening agents, flavouring agents, perfuming agents or mixtures thereof.

Injectable preparations, for example, sterile injections, aqueous suspensions may be formulated according to methods known to one of ordinary skill in the art, and in particular, using one or more suitable dispersing or wetting and suspending agents. Acceptable vehicles and solvents that may be employed include one or more of water, Ringer's solution, isotonic sodium chloride or mixtures thereof.

Dosage forms for tropical or transdermal administration of a compound of the present invention include ointments, pastes, creams, lotions, gels, powders, solutions, sprays, inhalants or patches. Active compounds can be admixed under sterile condition with one or more pharmaceutically acceptable carriers and optionally any preservatives or buffers as may be required. Ophthalmic formulations, eardrops, eye ointments, powders and solutions are also encompassed within the scope of this invention.

Pharmaceutical preparations may be in unit dosage form. In unit dosage form, the preparations can be subdivided into unit doses containing appropriate quantities of active components. Unit dosage forms can be packaged preparations containing discrete capsules, powders, in vials or ampoules, ointments, capsules, sachets, tablets, gels, creams or any combination and number of such packaged forms.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

### General Procedure

### Example 1

### Preparation of R—W—NH₂

(a) Preparation of 4-(3H)-imidazol[4,5-b]pyridin-3-yl-butylamine wherein W=—(CH₂)₄—

4-(3H)-imidazol[4,5-b]pyridin-3-yl-butylamine was prepared according to a procedure described in U.S. Pat. No. 5,635,485, which is incorporated herein in its entirety. In particular, 10.3 g of potassium carbonate were added to a solution of 5.95 g of 4-azabenzimidazole and 15.5 g of N-4-bromobutyl-phthalimide in 30 mL of dimethylformamide and the mixture was stirred for 20 hours at ambient temperature. The insoluble part was filtered off and rinsed with methylene chloride. The organic phase was washed with water, then dried over magnesium sulfate and evaporated. The oily residue obtained was washed with petroleum ether, then with isopropyl ether to obtain 16.3 g of a yellow solid, which was purified by chromatography on silica, eluting with a methylene chloride:acetone mixture to obtain 4.9 g of product (A) melting at 143° C.

A mixture of 32.86 g of product (A) obtained above, 697 mL of ethanol and 20 mL of hydrazine was refluxed for 19 hours. The mixture was allowed to return to ambient temperature, filtered, rinsed and evaporated to dryness. The residue was taken up in methylene chloride, filtered, rinsed and evaporated to dryness to obtain 18.87 g of the desired product.

### NMR CDCl₃-250 MHz

1.52 (m)-2.00 (m); 1.63 (wide s); 2.76 (t); 4.33 (t); 7.24 (dd, J= 8 and 5); 8.08 (dd, J=8 and 1.5); 8.40 (dd, J= 5 and 1.5); 8.08 (s).
(b) Preparation of N*1*-pyridin-4-yl-methylethane-1,2-diamine wherein W=—(CH₂)₂—NH—CH₂—

Pyridin-3-carboxaldehyde (46.7 mmol) was added to a solution of ethylene diamine (467.2 mmol) in methanol (50 mL) at 0-5° C. The reaction mixture was allowed to come to ambient temperature, sodium borohydride (46.7 mmol) was added in portions, and the reaction mixture was stirred for about 4 hours. The solvent was removed under reduced pressure to yield 5.2 g of crude product. The crude product was purified by silica gel column chromatography using 10% methanol:dichloromethane using 5% triethyl amine.
(c) wherein W=—(CH₂)₂—N(CH₃)—CH₂—

### Step I: Preparation of Methylpyridin-4-ylmethylamine

40% methyl amine (68.7 mmol) was added to a solution of pyridine 4-carbaldehyde (57.3 mmol) in methanol (90 mL) at 0-5° C. and stirred for about 15 minutes, followed by adding sodium borohydride (57.3 mmol). The reaction mixture was refluxed for about 1 hour, cooled to ambient temperature and solvent was removed under reduced pressure. Water was added to the resulting residue the mixture was extracted with dichloromethane. The dichloromethane layers were combined, mixed and washed successively with water and then brine, and dried over anhydrous sodium sulfate. Solvent was evaporated under reduced pressure to yield a crude product.

### Step II: Preparation of 2-[2-(methylpyridin-4-ylmethylamino) ethylisoindole-1,3-dione

Potassium carbonate (55.2 mmol) and 2-bromoethylfthalimide (66.2 mmol) was added to a solution of N*1*-pyridin-4-yl-methylethane-1,2-diamine (55.2 mmol) in dimethylformamide (50 mL). The reaction mixture was heated to 80° C. and stirred for about 16 hours. The reaction mixture was cooled to ambient temperature, poured into water and extracted with dichloromethane. The organic layer was washed with water, dried over anhydrous sodium sulfate and concentrated under reduced pressure to yield the title product.

### Step III: Preparation of N*1*-methyl-N*1*-pyridin-4-ylmethylethane-1,2-diamine

Hydrazine hydrate (202.8 mmol) was added to a solution of 2-[2-(methylpyridin-4-ylmethylamino)ethylisoindole-1,3-dione (20.28 mmol) in methanol (210 mL). The reaction mixture was refluxed for about 2 hours. The reaction mixture was cooled to ambient temperature and a white crystalline solid was filtered, washed with methanol (50 mL), and the solvent was removed under reduced pressure. Diethyl ether was added to the resulting residue and then stirred for about 30 minutes. A resulting solid was filtered and washed with ether. The filtrate was concentrated under reduced pressure to afford oily product.

### Example 2

### Synthetic Procedure of Scheme I

(a) Preparation of Compound of Formula III

Clarithromycin (25 g, 33.4 mmol) was added in portions to an aqueous solution of hydrochloric acid at ambient temperature. The reaction mixture was neutralized with solid sodium bicarbonate and the aqueous layer was extracted with ethyl acetate. The organic layer was washed successively with water and then brine, dried over anhydrous sodium sulfate and the solvent was removed under reduced pressure to yield a crude product. The crude product was crystallized from ethyl acetate and hexane to yield the title compound.
(b) Preparation of Compound of Formula IV

Benzoic anhydride (2.5 equiv.) followed by triethylamine (6 equiv.) was added to a solution of compound of Formula III (1 equiv.) in dichloromethane and stirred at ambient temperature for about 40 hours. The reaction was quenched by adding sodium bicarbonate solution. The aqueous layer was extracted with dichloromethane, washed successively with water and then brine, dried over anhydrous sodium sulfate and the solvent was removed under reduced pressure to yield a crude product. The crude product obtained was crystallized from ethyl acetate and hexane to yield the title compound.
(c) Preparation of Compound of Formula V

N-iodosuccinimide (2 equiv.) was added to a solution of compound of Formula IV (1 equiv.) in dry acetonitrile:dichloromethane (2:1) at about 0° C. The reaction mixture was allowed to come to ambient temperature while being stirred. A sodium bisulfite solution was added to the reaction mixture with stirring followed by adding a sodium carbonate solution with further stirring of the reaction mixture. Dichloromethane was evaporated under reduced pressure. The aqueous layer was extracted with ethyl acetate, washed successively with water and then brine, dried over anhydrous sodium sulfate and the solvent was removed under reduced pressure to yield a crude product. The crude product was purified by silica gel column chromatography (thoroughly neutralized with triethylamine) using 17-25% acetone in hexane to yield the title compound.
(d) Preparation of Compound of Formula VI

Compounds of Formula VI can be and were prepared by three different methods.

### Method A

Solid sodium hydrogen carbonate (5 equiv.) and ethyl iodide (6 equiv.) were added to a solution of compound of Formula V (1 equiv.) in acetonitrile under argon at ambient temperature and stirred for about 24 hours. The reaction was quenched by adding water. The reaction mixture was then diluted with ethyl acetate and washed with water followed by brine, dried over anhydrous sodium sulfate and concentrated under reduced pressure to yield a crude product. The crude product was purified by silica gel column chromatography (thoroughly neutralized with triethylamine) using 10-15% acetone in hexane to yield the title compound.

### Method B

The compound of Formula V (1 equiv.) in acetone and methanol was stirred at ambient temperature. A solution of acetic acid was added with stirring and sodium cyanoborohydride (2 equiv.) was then added with continued stirring. The solvent was evaporated under reduced pressure and a crude product was extracted with ethyl acetate. The ethyl acetate layer was combined and washed with water followed by brine, dried over anhydrous sodium sulfate and concentrated under reduced pressure. The crude product was purified by silica gel column chromatography (thoroughly neutralized with triethylamine) using 10-20% acetone in hexane to yield the title compound.

### Method C

(1-ethoxycyclopropoxy)trimethylsilane (3 equiv.) followed by glacial acetic acid (10 equiv.) were added to a solution of compound of Formula V (1 equiv.) in methanol at ambient temperature with stirring. The reaction mixture was cooled and sodium cyanoborohydride (5 equiv.) was then added. The reaction mixture was then stirred and refluxed. Methanol was removed under reduced pressure and water was then added. The organic layer was extracted with ethyl acetate. The ethyl acetate layer was combined, washed with water followed by brine, dried over anhydrous sodium sulfate and concentrated under reduced pressure. The crude product was purified by silica gel column chromatography (thoroughly neutralized with triethylamine) using 10-20% acetone in hexane to yield the title compound.
(e) Preparation of Compound of Formula VII

Triphosgene (1.5 equiv.) was added to a solution of compound of Formula VI (1 equiv.) in dichloromethane. Pyridine (15 equiv.) was then slowly added. After complete addition, the reaction mixture was stirred for about 4 hours and then quenched by adding ice-cold water. The reaction mixture was diluted with dichloromethane, washed with water followed by brine, dried over anhydrous sodium sulfate and concentrated under reduced pressure to yield the title compound.
(f) Preparation of Compound of Formula VIII

Tetramethyl guanidine (2.2 equiv.) was added to a solution of compound of Formula VII (1 equiv.) in dimethylformamide. The reaction mixture was heated to 70° C., stirred for about 10 hours, and then cooled to ambient temperature. The organic layer was extracted with ethyl acetate, washed with water followed by brine, dried over anhydrous sodium sulfate and concentrated under reduced pressure to yield the title compound.
(g) Preparation of Compound of Formula IX

Dess-Martine Periodinane (2.5 equiv.) was added to a solution of compound of Formula VIII (1 equiv.) in dichloromethane. The reaction mixture was refluxed for about one hour, cooled to ambient temperature, quenched by adding a saturated aqueous potassium carbonate solution followed by a saturated sodium thiosulfate solution, and the reaction mixture was then stirred. The aqueous layer was separated and extracted with dichloromethane. The dichloromethane layer was washed with water followed by brine, dried over anhydrous sodium sulfate and concentrated under reduced pressure to yield the title compound.
(h) Preparation of Compound of Formula X

N,N′-carbonyldiimidazole (3 equiv.) was added to a solution of compound of Formula IX (1 equiv.) in dimethylformamide:tetrahydrofuran (3:2) at ambient temperature. The reaction mixture was cooled, sodium hydride (3 equiv.) was added in portions and the reaction mixture was stirred. The reaction mixture was quenched by adding water. It was extracted with ethyl acetate. The organic layer was washed with water followed by brine, dried over anhydrous sodium sulfate and concentrated under reduced pressure to afford the desired product.
(i) Preparation of Compound of Formula XI

Compounds of Formula XI can be prepared by two different methods.

### Method A

Compound of Formula X (1 equiv.) and R—W—NH₂ (2 equiv.) were taken in water in acetonitrile, heated at 70° C. and stirred for about 20 hours. The reaction mixture was then cooled to ambient temperature and acetonitrile was removed under reduced pressure. The resulting residue was taken in ethyl acetate and washed with water followed by brine, dried over anhydrous sodium sulfate, and filtered. The filtrate thus obtained was collected and concentrated under reduced pressure. The crude product was purified by silica gel column chromatography (thoroughly neutralized with triethylamine) using 25-30% acetone in hexane to yield the title compound.

### Method B

Compound of Formula X (1 equiv.) and R—W—NH₂ (2 equiv.) were taken in dimethylformamide and heated at 70° C. and stirred for about 20 hours. The reaction mixture was cooled to ambient temperature and dimethylformamide was removed under reduced pressure. The resulting crude product was taken in ethyl acetate and washed with water followed by brine, dried over anhydrous sodium sulfate and concentrated under reduced pressure. The crude product was purified by silica gel column chromatography (thoroughly neutralized with triethylamine) using 25-30% acetone in hexane to yield the title compound.
(j) Preparation of Compound of Formula XII

Compounds of Formula XII can be and were prepared by two different methods.

### Method A

Sodium hydride (1.5 equiv.) was added in portions to a cold solution of compound of Formula XI (1 equiv.) in dimethylformamide. N-fluorobenzene sulfonimide (1.2 equiv.) was then added to the reaction mixture. The reaction mixture was quenched by adding water and then extracted with ethyl acetate. The oganic layer was washed with water followed by brine, dried over anhydrous sodium sulfate and concentrated under reduced pressure to yield the title compound.

### Method B

Potassium tert-butoxide (1.5 equiv.) was added to a solution of compound of Formula XI (1 equiv.) in tetrahydrofuran at −15° C. The reaction mixture was stirred for 20 minutes and N-fluorobenzene sulfonimide (1.2 equiv.) in tetrahydrofuran was then added. The reaction mixture was stirred at −15° C. for about 3 hours, quenched by adding water, and extracted with ethyl acetate. The organic layer was washed with water followed by brine, dried over anhydrous sodium sulfate and concentrated under reduced pressure to yield the title compound.
(k) Preparation of Compound of Formula XIII

Compound of Formula XII (560 mg, 0.6 mmol) was taken in methanol and refluxed. The reaction mixture was cooled to ambient temperature and methanol was removed under reduced pressure. A resulting solid product was purified over a silica gel column (thoroughly neutralized with triethylamine) using 20-40% acetone in hexane or 2-6% methanol in dichloromethane to yield the title compound.

### Example 3

### Synthetic Procedure of Scheme II

(a) Preparation of Compound of Formula XIV

Triphosgene (1.5 equiv) was added to a solution of compound of Formula IV (1 equiv) in dichloromethane and pyridine (15 equiv) was then slowly added. After complete addition, the reaction mixture was stirred for about 3-4 hours at 0° C. The reaction mixture was quenched by adding ice-cold water. The reaction mixture was diluted with dichloromethane and washed with water followed by brine, dried over anhydrous sodium sulfate and concentrated under reduced pressure to yield the title compound.
(b) Preparation of Compound of Formula XV

Tetramethyl guanidine (2.2 equiv.) was added to a solution of compound of Formula XIV (1 equiv.) in dimethylformamide. The reaction mixture was heated to 65-70° C. for about 3-4 hours and then cooled to ambient temperature. The organic layer was extracted with ethyl acetate and washed with water followed by brine, dried over anhydrous sodium sulfate and concentrated under reduced pressure to yield the title compound.
(c) Preparation of Compound of Formula XVI

Dess-Martine Periodinane (2.5 equiv.) was added to a solution of compound of Formula XV (1 equiv.) in dichloromethane and the reaction mixture was stirred at 30° C. for about 1 hour. The reaction mixture was quenched by adding saturated aqueous potassium carbonate solution followed by saturated sodium thiosulfate solution and the reaction mixture was stirred. The resulting aqueous layer was separated and extracted with dichloromethane. The dichloromethane layer was washed with water followed by brine, dried over anhydrous sodium sulfate and concentrated under reduced pressure to yield the title compound.
(d) Preparation of Compound of Formula XVII

N-iodosuccinimide (2 equiv.) was added to a solution of compound of Formula XVI (1 equiv.) in dry acetonitrile:dichloromethane (2:1). The reaction mixture was allowed to reach ambient temperature and stirred for about 3-4 hours. A sodium bisulfite solution was then added to the reaction mixture and stirred. Then sodium carbonate solution was added to the reaction mixture and stirring. Dichloromethane was evaporated under reduced pressure. The aqueous layer was extracted with ethyl acetate, washed successively with water followed by brine, dried over anhydrous sodium sulfate and the solvent was removed under reduced pressure to yield a crude product.
(e) Preparation of Compound of Formula Ix

Solid sodium hydrogen carbonate (5 equiv.) and ethyl iodide (6 equiv.) was added to a solution of compound of Formula XVII (1 equiv.) in acetonitrile under argon at ambient temperature and the reaction mixture was stirred for about 18-20 hours. The reaction mixture was quenched by adding water. The reaction mixture was then diluted with ethyl acetate, washed with water followed by brine, dried over anhydrous sodium sulfate and concentrated under reduced pressure to yield a crude product. The crude product was purified by silica gel column chromatography (thoroughly neutralized with triethylamine) using 10-15% acetone in hexane to yield the title compound.
(f) Preparation of compound of Formula XVIII

Compounds of Formula XVIII can be and were prepared by two different methods.

### Method A

Sodium hydride (1.5 equiv.) was added in portions to a solution of compound of Formula IX (1 equiv.) in dimethylformamide at 0° C. and then stirred for about 15 minutes. N-fluorobenzene sulfonimide (1.2 equiv.) was then added to the reaction mixture and stirred at 0° C. for about 3 hours. The reaction mixture was quenched by adding water and extracted with ethyl acetate. The organic layer was washed with water followed by brine, dried over anhydrous sodium sulfate and concentrated under reduced pressure to yield the title compound.

### Method B

Potassium fez-f-butoxide was added to a solution of compound of Formula IX (1 equiv.) in tetrahydrofuran at −15° C. and the reaction mixture was stirred for about 20 minutes. N-fluorobenzene sulfonimide (1.2 equiv.) in tetrahydrofuran was then added added to the reaction mixture and then stirred at −15° C. for about 2 hours, quenched by adding water and extracted with ethyl acetate. The organic layer was washed with water followed by brine, dried over anhydrous sodium sulfate and concentrated under reduced pressure to yield the title compound.
(g) Preparation of Compound of Formula XIX

N,N′-carbonyldiimidazole (3 equiv.) was added to a solution of compound of Formula XVIII (1 equiv.) in dimethylformamide:tetrahydrofuran (3:2) at ambient temperature. The reaction mixture was cooled to 0° C., sodium hydride (3 equiv.) was added in portions and the reaction mixture was stirred for about 30 minutes. The reaction mixture was quenched by adding water and then extracted with ethyl acetate. The ethyl acetate layer was washed with water followed by brine, dried over anhydrous sodium sulfate and concentrated under reduced pressure to yield the title compound.
(h) Preparation of Compound of Formula XII

Compound of Formula XIX (1 equiv.) and R—W—NH₂ (3 equiv.) were taken in 10% water in acetonitrile and heated to 65-70° C. for about 14 hours. The reaction mixture was cooled to ambient temperature and the acetonitrile-water mixture was removed under reduced pressure. The resulting residue was purified by silica gel column chromatography (thoroughly neutralized with triethylamine) using 25-30% acetone in hexane to yield the title compound.
(i) Preparation of Compound of Formula XIII

Compound of Formula XII was taken in methanol and refluxed for about 12 hours. The reaction mixture was cooled to ambient temperature and methanol was removed under reduced pressure. The resulting solid crude product was purified over a silica gel column using 2-6% methanol in dichloromethane to yield the title compound.

### Example 4

### Synthetic Procedure of Scheme III

(a) Preparation of Compound of Formula XX

Compounds of Formula XX can be and were prepared by two different methods.

### Method A

Sodium hydride (1.5 equiv.) was added in portions to a solution of compound of Formula XVI (1 equiv.) in dimethylformamide at 0° C. and the reaction mixture was stirred for about 15 minutes. N-fluorobenzene sulfonimide (1.2 equiv.) was then added and the reaction mixture was stirred at 0° C. for about 4 hours. The reaction mixture was quenched by adding water and extracted with ethyl acetate. The organic layer was washed with water followed by brine, dried over anhydrous sodium sulfate and concentrated under reduced pressure to yield the title compound.

### Method B

Potassium tert-butoxide (1.5 equiv) was added to a solution of compound of Formula XVI (1 equiv.) in tetrahydrofuran at −15° C. and the reaction mixture was stirred for about 20 minutes. N-fluorobenzene sulfonimide (1.2 equiv.) in tetrahydrofuran was then added and the reaction mixture was stirred at −15° C. for about 3 hours, quenched by adding water and extracted with ethyl acetate. The organic layer was washed with water followed by brine, dried over anhydrous sodium sulfate and concentrated under reduced pressure to yield the title compound.
(b) Preparation of compound of Formula XXI

N,N′-carbonyldiimidazole (3 equiv.) was added to a solution of compound of Formula XX (1 equiv.) in dimethylformamide:tetrahydrofuran (3:2) at ambient temperature. The reaction mixture was cooled to 0° C., sodium hydride (3 equiv.) was added in portions and the reaction mixture was stirred for about 30 minutes. The reaction was quenched by adding water and extracted with ethyl acetate. The ethyl acetate layer was washed with water followed by brine, dried over anhydrous sodium sulfate and concentrated under reduced pressure to yield the title compound.
(c) Preparation of Compound of Formula XXII

The compound of Formula XXI (1 equiv.) and R—W—NH₂ (2 equiv.) were taken in a mixture of 10% water in acetonitrile and heated to 65-70° C. for about 14 hours. The reaction mixture was cooled to ambient temperature and acetonitrile-water was removed under reduced pressure. The resulting residue was purified by silica gel column chromatography (thoroughly neutralized with triethylamine) using 25-30% acetone in hexane to yield the title compound.
(d) Preparation of compound of Formula XXIII

The compound of Formula XXII (560 mg, 0.6 mmol) was taken in methanol and refluxed. The reaction mixture was cooled to ambient temperature and methanol was evaporated under reduced pressure to yield the title compound.
(e) Preparation of Compound of Formula XXIV

N-iodosuccinimide (2 equiv.) was added to a solution of compound of Formula XXIII (1 equiv.) in dry acetonitrile and the reaction mixture was allowed to attain ambient temperature and stirred for 4 h. A sodium bisulfite solution was added to the reaction with stirring. A sodium carbonate solution was then added with stirring. The aqueous layer was extracted with ethyl acetate, washed successively with water followed by brine, dried over anhydrous sodium sulfate and the solvent was removed under reduced pressure to yield a crude product.
(f) Preparation of Compound of Formula XIII

Solid sodium hydrogen carbonate (5 equiv.) and ethyl iodide (6 equiv.) was added to a solution of compound of Formula XXIV (1 equiv.) in acetonitrile under argon at ambient temperature and stirred for about 20 hours. The reaction mixture was quenched by adding water. The reaction mixture was diluted with ethyl acetate and washed with water followed by brine, dried over anhydrous sodium sulfate and concentrated under reduced pressure to yield a crude product. The crude product was purified by silica gel column chromatography (thoroughly neutralized with triethylamine) using 30-35% acetone in hexane to yield the title compound.

### Example 5

### Synthetic Procedure for Scheme IV

(a) Preparation of Compound of Formula XXV

The compound of Formula XX was taken in methanol and refluxed. The reaction mixture was cooled to attain an ambient temperature and methanol was evaporated under reduced pressure. The crude product was purified by column chromatography.
(b) Preparation of Compound of Formula XXVI

N-iodosuccinimide (2 equiv.) was added to a solution of compound of Formula XXV (1 equiv.) in dry acetonitrile and the reaction mixture was allowed to attain ambient temperature and stirred for about 4 hours. A sodium bisulfite solution was added to the reaction mixture with stirring followed the addition of sodium carbonate solution with stirring. The aqueous layer was extracted with ethyl acetate, washed successively with water followed by brine, dried over anhydrous sodium sulfate and the solvent was removed under reduced pressure to yield a crude product.
(c) Preparation of compound of Formula XXVII

Solid sodium hydrogen carbonate (5 equiv.) and ethyl iodide (6 equiv.) was added to a solution of compound of Formula XXVI (1 equiv.) in acetonitrile under argon at ambient temperature and stirred for about 20 hours. The reaction mixture was quenched by adding water. The reaction mixture was diluted with ethyl acetate and washed with water followed by brine, dried over anhydrous sodium sulfate and concentrated under reduced pressure to yield a crude product. The crude product was purified by silica gel column chromatography (thoroughly neutralized with triethylamine) using 20-25% acetone in hexane to yield the title compound.
(d) Preparation of Compound of Formula XVIII

Potassium carbonate (2 equiv.) was added to a solution of compound of Formula XXVII (1 equiv.) in acetone and the reaction mixture was stirred for about 5 minutes. Aacetic anhydride (5 equiv.) was then added and the reaction mixture was stirred for about 24 hours at ambient temperature. The reaction mixture was poured into a chilled aqueous potassium carbonate solution and extracted with dichloromethane. The dichloromethane layer was washed with water followed by brine, dried over anhydrous sodium sulfate and concentrated to yield the title compound.
(e) Preparation of Compound of Formula XIX

N,N′-carbonyldiimidazole (3 equiv.) was added to a solution of compound of Formula XVIII (1 equiv.) in dimethylformamide:tetrahydrofuran (3:2) at ambient temperature. The reaction mixture was cooled to 0° C., sodium hydride (3 equiv.) was added in portions and the reaction mixture was stirred for about 30 minutes. The reaction mixture was quenched by adding water. This was extracted with ethyl acetate. Ethyl acetate layer was washed with water followed by brine, dried over anhydrous sodium sulfate and concentrated under reduced pressure to yield the title compound.
(f) Preparation of Compound of Formula XII

The compound of Formula XIX (1 equiv.) and R—W—NH₂ (2 equiv.) were taken in a mixture of 10% water in acetonitrile and heated to 65-70° C. for about 14 hours. The reaction mixture was cooled to ambient temperature and acetonitrile-water was removed under reduced pressure. The resulting residue was purified by silica gel column chromatography (thoroughly neutralized with triethylamine) using 25-30% acetone in hexane to yield the title compound.
(g) Preparation of Compound of Formula XIII

The compound of Formula XII was taken in methanol and refluxed. The reaction mixture was cooled to ambient temperature and methanol was removed under reduced pressure. The resulting solid crude product was purified by silica gel chromatography using 2-6% methanol in dichloromethane to yield the title compound.
(h) Preparation of a Hydrochloride Salt of Compound of Formula XIII

The compound of Formula XIII (0.09 mol) was taken in ethanol, cooled to 0° C. and 3N ethanolic hydrochloric acid (0.0945 mol) was added. The reaction mixture was stirred at 0° C. for about 2 hours and ethanol was removed under reduced pressure. The residue was dried under vacuum, triturated with diethyl ether and ether was decantated off to yield the title compound.

The following compounds were prepared following the above general procedures:
Compound No. 1: 2-α-Fluoro-5-O-(3′-N-desmethyl-3′-N-ethyl)-11,12-dideoxy-3-O-decladinosyl-6-O-methyl-3-oxo-12,11-[oxycarbonyl-((3-imidazol-1-yl)-propyl)-imino]erythromycin A; MS (+ ion mode): m/z 753.6 [M+1]
Compound No. 2: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((1H)-imidazo(-4,5-b)-pyridin-1-yl)-butyl)-imino)]erythromycin A; MS (+ ion mode): m/z 818.4 [M+1]
Compound No. 3: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((3H)-imidazo(4,5-b)-pyridin-1-yl)-butyl)-imino)]erythromycin A; MS (+ ion mode): m/z 818.3 [M+1]
Compound No. 4: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(benzoimidazol-1-yl)-butyl)-imino)]erythromycin A; MS (+ion mode): m/z 817.6 [M+1]
Compound No. 5: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(imidazol-1-yl)-butyl)-imino)]erythromycin A; MS (+ ion mode): m/z 767.6 [M+1]
Compound No. 6: 2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((3-imidazol-1-yl)-propyl)-imino]erythromycin A; MS (+ ion mode): m/z 765.6 [M+1]
Compound No. 7: 2-α-Fluoro-5-O-(3′-N-desmethyl-3′-N-isopropyl)-11,12-dideoxy-3-O-decladinosyl-6-O-methyl-3-oxo-12,11-[oxycarbonyl-((3-imidazol-1-yl)-propyl)-imino]erythromycin A; MS (+ ion mode): m/z 767.5 [M+1]
Compound No. 8: 2-α-Fluoro-5-O-(3′-N-desmethyl-3′-N-propyl)-11,12-dideoxy-3-O-decladinosyl-6-O-methyl-3-oxo-12,11-[oxycarbonyl-((3-imidazol-1-yl)-propyl)-imino]erythromycin A; MS (+ ion mode): m/z 767.6 [M+1]
Compound No. 9: 2-α-Fluoro-5-O-(3′-N-desmethyl-3′-N-propyl)-11,12-dideoxy-3-O-decladinosyl-6-O-methyl-3-oxo-12,11-[oxycarbonyl-((4-(benzoimidazol-1-yl)-butyl)-imino)]erythromycin A; MS (+ ion mode): m/z 831.3 [M+1]
Compound No. 10: 2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-pyrrolo-(2,3-b)pyridin-1-yl)-butyl)-imino)]erythromycin A; MS (+ ion mode): m/z 829.4 [M+1]
Compound No. 11: 2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-(pyrrolo-(2,3-b)pyridin-1-yl)-butyl)-imino)]erythromycin A; MS (+ ion mode): m/z 831.6 [M+1]
Compound No. 12: 2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-(benzoimidazol-1-yl)-butyl)-imino)]erythromycin A; MS (+ ion mode): m/z 831.4 [M+1]
Compound No. 13: 2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-(benzoimidazol-1-yl)-butyl)-imino)]erythromycin A; MS (+ ion mode): m/z 829.7 [M+1]
Compound No. 14: 2-α-Fluoro-5-O-(3′-N-desmethyl-3′-N-n-propyl)-11,12-dideoxy-3-O-decladinosyl-6-O-methyl-3-oxo-12,11-[oxycarbonyl-((4-(imidazol-1-yl)-butyl)-imino)]erythromycin A; MS (+ ion mode): m/z 781.5 [M+1]
Compound No. 15: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyrrolo-(2,3-b)pyridin-1-yl)-butyl)-imino)]erythromycin A; MS (+ ion mode): m/z 817.6 [M+1]
Compound No. 16: 2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-((1H)-imidazo(4,5-b)-pyridin-1-yl)-butyl)-imino)]erythromycin A; MS (+ ion mode): m/z 832.9 [M+1]
Compound No. 17: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(purin-9-yl)-butyl)-imino)]erythromycin A; MS (+ ion mode): m/z 820.0 [M+1]
Compound No. 18: 2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-((3H)-imidazo(4,5-b)-pyridin-1-yl)-butyl)-imino)]erythromycin A; MS (+ ion mode): m/z 830.4 [M+1]
Compound No. 19: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-pyridin-3-yl-imidazol-1-yl)-butyl)-imino)]erythromycin A; MS (+ ion mode): m/z 844.55 [M+1]
Compound No. 20 :2α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-quinolin-4-ylmethyl)-2-aminoethyl)-imino]erythromycin A; MS (+ ion mode): m/z 843.90 [M+1]
Compound No. 21: 2-α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-pyridine-3-ylmethyl)-2-aminoethyl)-imino]erythromycin A; MS (+ ion mode): m/z 794.00 [M+1]
Compound No. 22: 2-α-Fluoro-5-O-(3′-N-desmethyl-3*-N-ethyl)-11,12-dideoxy-3-O-decladinosyl-6-O-methyl-3-oxo-12,11-[oxycarbonyl-((N′-methyl-N-pyridine-4-ylmethyl)-2-aminoethyl)-imino]erythromycin A; MS (+ ion mode): m/z 794.00 [M+1]
Compound No. 23: 2-α-Fluoro-5-O-(3′-N-desmethyl-3′-N-ethyl)-11,12-dideoxy-3-O-decladinosyl-6-O-methyl-3-oxo-12,11-[oxycarbonyl-((N′-methyl-N′-pyridine-2-ylmethyl)-2-aminoethyl)-imino]erythromycin A; MS (+ ion mode): m/z 794.01 [M+1]
Compound No. 24: 2-α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-pyridine-2-ylmethyl)-2-aminoethyl)-imino]erythromycin A; MS (+ ion mode): m/z 805.58 [M+1]
Compound No. 25: 2-α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-pyridine-3-ylmethyl)-2-aminoethyl)-imino]erythromycin A; MS (+ ion mode): m/z 805.45 [M+1]
Compound No. 26: 2-α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-pyridine-4-ylmethyl)-2-aminoethyl)-imino]erythromycin A; MS (+ ion mode): m/z 805.40 [M+1]
Compound No. 27: 2-α-Fluoro-5-O-(3′-N-desmethyl-3′-N-allyl)-11,12-dideoxy-3-O-decladinosyl-6-O-methyl-3-oxo-12,11-[oxycarbonyl-((N′-methyl-N′-quinoline-4-ylmethyl)-2-aminoethyl)-imino]erythromycin A; MS (+ ion mode): m/z 855.59 [M+1]
Compound No. 28: 2-α-Fluoro-5-O-(3′-N-desmethyl-3′-N-ethyl)-11,12-dideoxy-3-O-decladinosyl-6-O-methyl-3-oxo-12,11-[oxycarbonyl-((N′-pyridine-3-ylmethyl)-2-aminoethyl)-imino]erythromycin A; MS (+ ion mode): m/z 779.71 [M+1]
Compound No. 29: 2-α-Fluoro-5-O-(3′-N-desmethyl-3′-N-allyl)-11,12-dideoxy-3-O-decladinosyl-6-O-methyl-3-oxo-12,11-[oxycarbonyl-((N′-acetyl-N′-pyridine-3-ylmethyl)-2-aminoethyl)-imino]erythromycin A; MS (+ ion mode): m/z 833.78 [M+1]
Compound No. 30: 2-α-Fluoro-5-O-(3′-N-desmethyl-3″-N-allyl)-11,12-dideoxy-3-O-decladinosyl-6-O-methyl-3-oxo-12,11-[oxycarbonyl-((N′-pyridine-3-ylmethyl)-2-aminoethyl)-imino]erythromycin A; MS (+ ion mode): m/z 791.73 [M+1]
Compound No. 31: 2-α-Fluoro-5-O-(3′-N-desmethyl-3′-N-ethyl)-11,12-dideoxy-3-O-decladinosyl-6-O-methyl-3-oxo-12,11-[oxycarbonyl-((N′-acetyl-N′-pyridine-3-ylmethyl)-2-aminoethyl)-imino]erythromycin A; MS (+ ion mode): m/z 821.76 [M+1]
Compound No. 32: 2-α-Fluoro-5-O-(3′-N-desmethyl-3′-N-ethyl)-11,12-dideoxy-3-O-decladinosyl-6-O-methyl-3-oxo-12,11-[oxycarbonyl-((N′,N′-di-pyridine-3-ylmethyl)-2-aminoethyl)-imino]erythromycin A; MS (+ ion mode): m/z 870.54 [M+1]
Compound No. 33: 2-α-Fluoro-5-O-(3′-N-desmethyl-3′-N-allyl)-11,12-dideoxy-3-O-decladinosyl-6-O-methyl-3-oxo-12,11-[oxycarbonyl-((N′,N′-di-pyridine-3-ylmethyl)-2-aminoethyl)-imino]erythromycin A; MS (+ ion mode): m/z 882.69 [M+1]
Compound No. 34: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-(quinolin-3-yl)-butyl)-imino)]erythromycin A; MS (+ ion mode): m/z 840.7 [M+1]
Compound No. 35: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(quinolin-8-yl)-butyl)-imino)]erythromycin A; MS (+ ion mode): m/z 829[M+1]
Compound No. 36: 2-α-Fluoro-5-O-(3′-N-desmethyl-3″-N-ethyl)-11,12-dideoxy-3-O-decladinosyl-6-O-methyl-3-oxo-12,11-[oxycarbonyl-((4-(pyridin-2-yl)-butyl)-imino)]erythromycin A; MS (+ ion mode): m/z 778 [M+1]
Compound No. 37: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-3-yl)-butyl)-imino)]erythromycin A; MS (+ ion mode): m/z 778 [M+1]
Compound No. 38: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(pyridin-4-yl)-butyl)-imino)]erythromycin A; MS (+ ion mode): m/z 778 [M+1]
Compound No. 39: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-(pyridin-2-yl)-butyl)-imino)]erythromycin A; MS (+ ion mode): m/z 789 [M+1]
Compound No. 40: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-(pyridin-4-yl)-butyl)-imino)]erythromycin A; MS (+ ion mode): m/z 789 [M+1]
Compound No. 41: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-(pyridin-3-yl)-pentyl)-imino]erythromycin A; MS (+ ion mode): m/z 804.77 [M+1]
Compound No. 42: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-(pyridin-3-yl)-butyl)-imino)]erythromycin A; MS (+ ion mode): m/z 790.50 [M+1]
Compound No. 43: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(indol-1-yl)-butyl)-imino)]erythromycin A; MS (+ ion mode): m/z 816.8 [M+1]
Compound No. 44: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A; MS (+ ion mode): m/z 843.9 [M+1]
Compound No. 45: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((5-nitro)-indol-1-yl)-butyl)-imino)]erythromycin A; MS (+ ion mode): m/z 861.8 [M+1]
Compound No. 46: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((1H)-imidazo[4,5-c]pyridin-1-yl)-butyl)-imino)]erythromycin A; MS (+ ion mode): m/z 819.1 [M+1]
Compound No. 47: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(benzotriazol-2-yl)-butyl)-imino)]erythromycin A; MS (+ ion mode): m/z 818.9 [M+1]
Compound No. 48: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((3H)-imidazo[4,5-c]pyridin-3-yl)-butyl)-imino)]erythromycin A; MS (+ ion mode): m/z 819 [M+1]
Compound No. 49: 2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A; MS (+ ion mode): m/z 855.8 [M+1]
Compound No. 50: 2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-(benzotriazol-1-yl)-butyl)-imino)]erythromycin A; MS (+ ion mode): m/z 830.7 [M+1]
Compound No. 51: 2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-(benzotriazol-2-yl)-butyl)-imino)]erythromycin A; MS (+ ion mode): m/z 830.9 [M+1]
Compound No. 52: 2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-((1H)-imdazo[4,5-b]pyridin-1-yl)-butyl)-imino)]erythromycin A; MS (+ ion mode): m/z 830.9 [M+1]
Compound No. 53: 2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-(indol-1-yl)-butyl)-imino)]erythromycin A; MS (+ ion mode): m/z 828.9 [M+1]
Compound No. 54: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-(benzotriazol-1-yl)-butyl)-imino)]erythromycin A; MS (+ ion mode): m/z 830.9 [M+1]
Compound No. 55: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-(benzoimidazol-1-yl)-butyl)-imino)]erythromycin A; MS (+ ion mode): m/z 829.9 [M+1]
Compound No. 56: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A; MS (+ ion mode): m/z 830.9 [M+1]
Compound No. 57: 2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A; MS (+ ion mode): m/z 858.1 [M+1]
Compound No. 58: 2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-(benzotriazol-2-yl)-butyl)-imino)]erythromycin A; MS (+ ion mode): m/z 832.8 [M+1]
Compound No. 59: 2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-(indol-1-yl)-butyl)-imino)]erythromycin A; MS (+ ion mode): m/z 830.9 [M+1]
Compound No. 60: 2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A; MS (+ ion mode): m/z 832.9 [M+1]
Compound No. 61: 2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-((5-nitro)-indol-1-yl)-butyl)-imino)]erythromycin A; MS (+ ion mode): m/z 875.38 [M+1]
Compound No. 62: 2-α-Fluoro-5-O-(3′-N-desmethyl-3′-N-cyclopropyl)-11,12-dideoxy-3-O-decladinosyl-6-O-methyl-3-oxo-12,11-[oxycarbonyl-((4-((5-nitro)-indol-1-yl)-butyl)-imino)]erythromycin A; MS (+ ion mode): m/z 873.54 [M+1]
Compound No. 63: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((5-fluoro)-indol-1-yl)-butyl)-imino)]erythromycin A; MS (+ ion mode): m/z 834.57 [M+1]
Compound No. 64: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(benzotriazol-1-yl)-butyl)-imino)]erythromycin A; MS (+ ion mode): m/z 818.9 [M+1]
Compound No. 65: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-methyl)-benzoimidazol-1-yl)-butyl)-imino)]erythromycin A; MS (+ ion mode): m/z 831.57 [M+1]
Compound No. 66: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-ethyl)-benzoimidazol-1-yl)-butyl)-imino)]erythromycin A; MS (+ ion mode): m/z 845.58 [M+1]
Compound No. 67: 2-α-Fluoro-5-O-(3′-N-desmethyl-3′-N-isopropyl)-11,12-dideoxy-3-O-decladinosyl-6-O-methyl-3-oxo-12,11-[oxycarbonyl-((4-(benzotriazol-1-yl)-butyl)-imino)]erythromycin A; MS (+ ion mode): m/z 832.54 [M+1]
Compound No. 68: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((2-ethyl)-benzoimidazol-1-yl)-butyl)-imino)]erythromycin A; MS (+ ion mode): m/z 857.56 [M+1]
Compound No. 69: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-pyridin-3-yl-imidazol)-1-yl)-butyl)-imino)]erythromycin A; MS (+ ion mode): m/z 856.64 [M+1]
Compound No. 70: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((2-methyl)-benzoimidazol-1-yl)-butyl)-imino)]erythromycin A; MS (+ ion mode): m/z 843.53 [M+1]
Compound No. 71: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A; MS (+ ion mode): m/z 829.59 [M+1]
Compound No. 72: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((1H)-imidazo[4,5-b]pyridin-1-yl)-butyl)-imino)]erythromycin A; MS (+ ion mode): m/z 830.6 [M+1]
Compound No. 73: 2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A; MS (+ ion mode): m/z 836.65 [M+1]
Compound No. 74: 2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((5-nitro)-indol-1-yl)-butyl)-imino)]erythromycin A; MS (+ ion mode): m/z 879.5 [M+1]
Compound No. 75: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((2-pyridin-3-ylmethyl)-benzoimidazol-1-yl)-butyl)-imino)]erythromycin A; MS (+ ion mode): m/z 920.74 [M+1]
Compound No. 76: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-pyridin-3-ylmethyl)-benzoimidazol-1-yl)-butyl)-imino)]erythromycin A; MS (+ ion mode): m/z 908.71 [M+1]
Compound No. 77: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((2-phenyl)-imidazol-1-yl)-butyl)-imino)]erythromycin A; MS (+ ion mode): m/z 855.6 [M+1]
Compound No. 78: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((5-fluoro)-indol-1-yl)-butyl)-imino)]erythromycin A; MS (+ ion mode): m/z 846.57 [M+1]
Compound No. 79: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((5,6-dimethyl)-benzoimidazol-1-yl)-butyl)-imino)]erythromycin A; MS (+ ion mode): m/z 857.89 [M+1]
Compound No. 80: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((5,6-dimethyl)-benzoimidazol-1-yl)-butyl)-imino)]erythromycin A; MS (+ ion mode): m/z 845.8 [M+1]
Compound No. 81: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((3H)-imidazo[4,5-c]pyridin-3-yl)-butyl)-imino)]erythromycin A; MS (+ ion mode): m/z 831.7 [M+1]
Compound No. 82: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((2-methyl)-(3H)-imidazo[4,5-b]pyridin-3-yl)-butyl)-imino)]erythromycin A; MS (+ ion mode): m/z 844.67 [M+1]
Compound No. 83: 2-α-Fluoro-5-O-(3′-N-desmethyl-3*-N-ethyl)-11,12-dideoxy-3-O-decladinosyl-6-O-methyl-3-oxo-12,11-[oxycarbonyl-((4-((2-methyl)-(3H)-imidazo[4,5-b]pyridin-3-yl)-butyl)-imino)]erythromycin A; MS (+ ion mode): m/z 832.68 [M+1]
Compound No. 84: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((1H)-imidazo[4,5-c]pyridin-1-yl)-butyl)-imino)]erythromycin A; MS (+ ion mode): m/z 830.78 [M+1]
Compound No. 85: 2-α-Fluoro-5-O-(3′-N-desmethyl-3′-N-ethyl)-11,12-dideoxy-3-O-decladinosyl-6-O-methyl-3-oxo-12,11-[oxycarbonyl-((9-(4-amino-butyl)-9H-purin-6-yl)-imino]erythromycin A; MS (+ ion mode): m/z 834.78 [M+1]
Compound No. 86: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-phenyl)-imidazol-1-yl)-butyl)-imino)]erythromycin A; MS (+ ion mode): m/z 843.8 [M+1]
Compound No. 87: 2-α-Fluoro-5-O-(3′-N-desmethyl-3′-N-acetyl)-11,12-dideoxy-3-O-decladinosyl-6-O-methyl-3-oxo-12,11-[oxycarbonyl-((4-(benzoimidazol-1-yl)-butyl)-imino)]erythromycin A; MS (+ ion mode): m/z 831.82 [M+1]
Compound No. 88: 2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A; MS (+ ion mode): m/z 844.85 [M+1]
Compound No. 89: 2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-(benzoimidazol-1-yl)-butyl)-imino)]erythromycin A; MS (+ ion mode): m/z 843.86 [M+1]
Compound No. 90: 2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A; MS (+ ion mode): m/z 843.8 [M+1]
Compound No. 91: 2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A; MS (+ ion mode): m/z 835.83 [M+1]
Compound No. 92: 2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-(benzotriazol-1-yl)-butyl)-imino)]erythromycin A; MS (+ ion mode): m/z 836.74 [M+1]
Compound No. 93: 2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-(benzoimidazol-1-yl)-butyl)-imino)]erythromycin A; MS (+ ion mode): m/z 835.77 [M+1]
Compound No. 94: 2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A; MS (+ ion mode): m/z 861.8 [M+1]
Compound No. 95: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((2-trifluoromethyl)-benzoimidazol-1-yl)-butyl)-imino)]erythromycin A; MS (+ ion mode): m/z 885.75 [M+1]
Compound No. 96: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4,5-diphenyl)-imidazol-1-yl)-butyl)-imino)]erythromycin A; MS (+ ion mode): m/z 919.15 [M+1]
Compound No. 97: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4,5-diphenyl)-imidazol-1-yl)-butyl)-imino)]erythromycin A; MS (+ ion mode): m/z 931.85 [M+1]
Compound No. 98: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A; MS (+ ion mode): m/z 855.75 [M+1]
Compound No. 99: 2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A; MS (+ ion mode): m/z 869.85 [M+1]
Compound No. 100: 2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-(benzotriazol-1-yl)-butyl)-imino)]erythromycin A; MS (+ ion mode): m/z 844.8 [M+1]
Compound No. 101: 2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((4-pyridin-3-yl)-imidazol-1-yl)-butyl)-imino)]erythromycin A; MS (+ ion mode): m/z 870.82 [M+1]
Compound No. 102: 2-α-Fluoro-5-O-(3′-N-desmethyl-3′-N-acetyl)-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A; MS (+ ion mode): m/z 831.78 [M+1]
Compound No. 103: 2-α-Fluoro-5-O-(3′-N-desmethyl-3′-N-acetyl)-11,12-dideoxy-3-O-decladinosyl-6-O-methyl-3-oxo-12,11-[oxycarbonyl-((4-((4-phenyl)-imidazol-1-yl)-butyl)-imino)]erythromycin A; MS (+ ion mode): m/z 857.83 [M+1]
Compound No. 104: 2-α-Fluoro-5-O-(3′-N-desmethyl-3′-N-acetyl)-11,12-dideoxy-3-O-decladinosyl-6-O-methyl-3-oxo-12,11-[oxycarbonyl-((4-((1H)-imidazo[4,5-b]pyridin-1-yl)-butyl)-imino)]erythromycin A; MS (+ ion mode): m/z 832.77 [M+1]
Compound No. 105: 2-α-Fluoro-5-O-(3′-N-desmethyl-3′-N-acetyl)-11,12-dideoxy-3-O-decladinosyl-6-O-methyl-3-oxo-12,11-[oxycarbonyl-((4-((3H)-imidazo[4,5-b]pyridin-3-yl)-butyl)-imino)]erythromycin A; MS (+ ion mode): m/z 832.77 [M+1]
Compound No. 106: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((3,5-diphenyl)-pyrazol-1-yl)-butyl)-imino)]erythromycin A; MS (+ ion mode): m/z 931.85 [M+1]
Compound No. 107: 2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((4-pyridin-3-yl)-imidazol-1-yl)-butyl)-imino)]erythromycin A; MS (+ ion mode): m/z 862.69 [M+1]
Compound No. 108: 2-α-Fluoro-5-O-(3′-N-desmethyl-3*-N-2-fluoroethyl)-11,12-dideoxy-3-O-decladinosyl-6-O-methyl-3-oxo-12,11-[oxycarbonyl-((4-((1H)-imidazo[4,5-b]pyridin-1-yl)-butyl)-imino)]erythromycin A; MS (+ ion mode): m/z 834 [M+1]
Compound No. 109: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((3,5-diphenyl)-pyrazol-1-yl)-butyl)-imino)]erythromycin A; MS (+ ion mode): m/z 919.85 [M+1]
Compound No. 110: 2-α-Fluoro-5-O-(3′-N-desmethyl-3′-N-cyclopropyl methyl)-11,12-dideoxy-3-O-decladinosyl-6-O-methyl-3-oxo-12,11-[oxycarbonyl-((4-((1H)-imidazo[4,5-b]pyridin-1-yl)-butyl)-imino)]erythromycin A; MS (+ ion mode): m/z 844.8 [M+1]
Compound No. 111: 2-α-Fluoro-5-O-(3′-N-desmethyl-3′-N-acetyl)-11,12-dideoxy-3-O-decladinosyl-6-O-methyl-3-oxo-12,11-[oxycarbonyl-((4-((4-pyridin-3-yl)-imidazo)-1-yl)-butyl)-imino)]erythromycin A; MS (+ ion mode): m/z 858.64 [M+1]
Compound No. 112: 2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((4-(2,4-difluoro)-phenyl)-imidazol-1-yl)-butyl)-imino)]erythromycin A; MS (+ ion mode): m/z 897.6 [M+1]
Compound No. 113: 2-α-Fluoro-5-O-(3′-N-desmethyl-3′-N-2-ethyl)-11,12-dideoxy-3-O-decladinosyl-6-O-methyl-3-oxo-12,11-[oxycarbonyl-((4-((4-(2,4-difluoro)-phenyl)-imidazol)-1-yl)-butyl)-imino)]erythromycin A; MS (+ ion mode): m/z 879.62 [M+1]
Compound No. 114: 2-α-Fluoro-5-O-(3′-N-desmethyl-3′-N-2-allyl)-11,12-dideoxy-3-O-decladinosyl-6-O-methyl-3-oxo-12,11-[oxycarbonyl-((4-((4-(2,4-difluoro)-phenyl)-imidazol)-1-yl)-butyl)-imino)]erythromycin A; MS (+ ion mode): m/z 891.65[M+1]
Compound No. 115: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-furan-3-yl)-imidazol-1-yl)-butyl)-imino)]erythromycin A; MS (+ ion mode): m/z 833.56[M+1]
Compound No. 115a: 2-α-Fluoro-5-O-(3′-N-desmethyl-3′-N-ethyl)-11,12-dideoxy-3-O-decladinosyl-6-O-methyl-3-oxo-12,11-[oxycarbonyl(4-((4-furan-3-yl)-imidazol-1-yl)-butyl)-imino)]erythromycin A hydrochloride salt; M.Pt: 185-188° C. (decomposed)
Compound No. 116: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-phenyl-pyrazol-1-yl)-butyl)-imino))]erythromycin A; Mass: m/z 855.35 [M⁺+1]
Compound No. 117: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-pyridin-3-yl-1H-imidazol-1-yl)-pentyl)-imino)]erythromycin A; Mass: m/z 858.38 [M⁺+1]
Compound No. 118: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-pyridin-4-yl-1H-imidazol-1-yl)-butyl)-imino))]erythromycin A; Mass: m/z 844.41 [M⁺+1]
Compound No. 119: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-pyridin-4-yl-1H-imidazol-1-yl)-butyl)-imino))]erythromycin A; Mass: m/z 856.39 [M⁺+1]
Compound No. 120: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-phenyl-pyrazol-1-yl)-butyl)-imino)) erythromycin A; Mass: m/z 843.55 [M⁺+1]
Compound No. 121: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((indazol-2-yl)-butyl)-imino))]erythromycin A; Mass: m/z 817.54 [M⁺+1]
Compound No. 122: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((indazol-2-yl)-butyl)-imino))]erythromycin; Mass: m/z 829.60 [M⁺+1]
Compound No. 123: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((3-pyridin-3-yl-pyrazol-1-yl)-butyl)-imino))]erythromycin A; Mass: m/z 844.54 [M⁺+1]
Compound No. 124: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((indazol-1-yl)-butyl)-imino))]erythromycin A; Mass: m/z 817.52 [M⁺+1]
Compound No. 125: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-pyrazin-2-yl-1H-imidazol-1-yl)-butyl)-imino))]erythromycin A; Mass: m/z 845.56 [M⁺+1]
Compound No. 126: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-thiophen-3-yl-1H-imidazol-1-yl)-butyl)-imino))]erythromycin A; Mass: m/z 849.45 [M⁺+1]
Compound No. 127: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-thiophen-3-yl-1H-imidazol-1-yl)-butyl)-imino))]erythromycin A; Mass: m/z 861.45 [M⁺+1]
Compound No. 128: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-quinolin-3-yl-1H-imidazol-1-yl)-butyl)-imino))]erythromycin A; Mass: m/z 894.60 [M⁺+1]
Compound No. 129: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-(indazol-1-yl)-butyl)-imino))]erythromycin A; Mass: m/z 829.49 [M⁺+1]
Compound No. 130: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-pyrimidin-5-yl-1H-imidazol-1-yl)-butyl)-imino))]erythromycin A; Mass: m/z 845.46 [M⁺+1]
Compound No. 131: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-pyrimidin-5-yl-1H-imidazol-1-yl)-butyl)-imino))]erythromycin A; Mass: m/z 857.48 [M⁺+1]
Compound No. 132: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-furan-2-yl-1H-imidazol-1-yl)-butyl)-imino))]erythromycin A; Mass: m/z 833.56 [M⁺+1]
Compound No. 133: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-thiophen-2-yl-1H-imidazol-1-yl)-butyl)-imino))]erythromycin A; Mass: m/z 849.57 [M⁺+1]
Compound No. 134: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(4-chloro-phenyl)-1H-imidazol-1-yl)-butyl)-imino))]erythromycin A; Mass: m/z 877.40 [M⁺+1]
Compound No. 135: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-(4-chloro-phenyl)-1H-imidazol-1-yl)-butyl)-imino))]erythromycin A; Mass: m/z 889.40 [M⁺+1]
Compound No. 136: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-pyridin-2-yl-1H-imidazol-1-yl)-butyl)-imino))]erythromycin A; Mass: m/z 844.46 [M⁺+1]
Compound No. 137: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((4-pyridin-2-yl-1H-imidazol-1-yl)-butyl)-imino))]erythromycin A; Mass: m/z 856.44 [M⁺+1]
Compound No. 138: 2-α-Fluoro-5-O-(3′-N-desmethyl-3′-N-allyl)-11,12-dideoxy-3-O-decladinosyl-6-O-methyl-3-oxo-12,11-[oxycarbonyl-((9-(4-amino-butyl)-9H-purin-6-yl)-imino)]erythromycin A; Mass: m/z 846.45 [M⁺+1]
Compound No. 139: 2-α-Fluoro-5-O-(3′-N-desmethyl-3′-N-allyl)-11,12-dideoxy-3-O-decladinosyl-6-O-methyl-3-oxo-12,11-[oxycarbonyl-((4-((3-pyridin-3-yl-1H-pyrazol-1-yl)-butyl)-imino))]erythromycin A; Mass: m/z 856.61[M⁺+1]
Compound No. 140: 2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((4-(4-chloro-phenyl)-1H-imidazol-1-yl)-butyl)-imino))]erythromycin A; Mass: m/z 895.49 [M⁺+1]
Compound No. 141: 2-α-Fluoro-5-O-(3′-N-desmethyl-3′-N-2-fluoroethyl)-11,12-dideoxy-3-O-decladinosyl-6-O-methyl-3-oxo-12,11-[oxycarbonyl-((4-((4-pyridin-2-yl)-1H-imidazol-1-yl)-butyl)-imino))]erythromycin A; Mass: m/z 862.49 [M⁺+1]
Compound No. 142: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-Imidazo[4,5-b]pyridine-2-yl)-butyl)-imino)erythromycin A; Mass: m/z 818.95 [M⁺+1]
Compound No. 143: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Tetrazol-1-yl-imidazol-1-yl)-butyl)-imino)erythromycin A; Mass: m/z 835.44 [M⁺+1]
Compound No. 144: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(1H-Benzoimidazol-2-yl)-butyl)-imino)erythromycin A; Mass: m/z 817.45 [M⁺+1]
Compound No. 145: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[4-(6-Fluoro-pyridin-3-yl)-imidazol-1-yl]-butyl)-imino)erythromycin A; Mass: m/z 862.39 [M⁺+1]
Compound No. 146: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-Imidazo[4,5-c]pyridine-2-yl)-butyl)iminoerythromycin A; Mass: m/z 818.4 [M⁺+1]
Compound No. 147: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-[1,2,4]Trizol-1-yl-phenyl)-butyl)-imino)erythromycin A; Mass: m/z 844.4 [M⁺+1]
Compound No. 148: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Imidazol-1-yl-phenyl)-butyl)-imino)erythromycin A; Mass: m/z 843.5 [M⁺+1]
Compound No. 149: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[4-(6-Chloro-pyridin-3-yl)-imidazol-1-yl]-butyl)-imino)erythromycin A; Mass: m/z 878.4 [M⁺+1]
Compound No. 150: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(3-[1-(4-Amino-butyl)-1H-imidazol-4-yl]-phenyl)iminoerythromycin A; Mass: m/z 858.5 [M⁺+1]
Compound No. 151: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Pyridin-3-yl-thiazol-2-yl)-butyl)-imino)erythromycin A; Mass: m/z 861.3 [M⁺+1]
Compound No. 152: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(3-[2-(4-Amino-butyl)-thiazol-4-yl]-phenyl)iminoerythromycin A; Mass: m/z 875.4 [M⁺+1]
Compound No. 153: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Pyrazol-1-yl-imidazol-1-yl)-butyl)-imino)erythromycin A; Mass: m/z 833.4 [M⁺+1]
Compound No. 154: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-([1,4′]-Bipyrazolyl-1′-yl)-butyl) iminoerythromycin A; Mass: m/z 833.4 [M⁺+1]
Compound No. 155: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Imidazol-1-yl-pyrazol-1-yl)-butyl)-imino)erythromycin A; Mass: m/z 833.4 [M⁺+1]
Compound No. 156: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Pyrazol-1-yl-imidazol-1-yl)-butyl)-imino)erythromycin A; Mass: m/z 833.4 [M⁺+1]
Compound No. 157: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[3,3′]Bithiophenyl-5-yl-butyl)-imino)erythromycin A; Mass: m/z 865.3 [M⁺+1]
Compound No. 158: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[2,3′]Bithiophenyl-5′-yl-butyl)-imino)erythromycin A; Mass: m/z 865.3 [M⁺+1]
Compound No. 159: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Furan-2-yl-thiaophen-2-yl)-butyl)-imino)erythromycin A; Mass: m/z 849.4 [M⁺+1]
Compound No. 160: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Oxazol-5-yl-imidazol-1-yl)-butyl)-imino)erythromycin A; Mass: m/z 834.4 [M⁺+1]
Compound No. 161: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3-Pyrrol-1-yl-[1,2,4]triazol-1-yl)-butyl) iminoerythromycin A; Mass: m/z 833.4 [M⁺+1]
Compound No. 162: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-Thiophen-2-yl-tetrazol-2-yl)-butyl)-imino)erythromycin A; Mass: m/z 851.4 [M⁺+1]
Compound No. 163: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Thiophen-3-yl-pyrazol-1-yl)-butyl)-imino)erythromycin A; Mass: m/z 849.4 [M⁺+1]
Compound No. 164: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Furan-3-yl-pyrazol-1-yl)-butyl)-imino)erythromycin A; Mass: m/z 833.4 [M⁺+1]
Compound No. 165: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Furan-2-yl-pyrazol-1-yl)-butyl)-imino)erythromycin A; Mass: m/z 833.4 [M⁺+1]
Compound No. 166: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-Phenyl-tetrazol-2-yl)-butyl)-imino)erythromycin A; Mass: m/z 845.4 [M⁺+1]
Compound No. 167: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[5-(4-Methoxy-phenyl)-tetrazol-2-yl]-butyl)-imino)erythromycin A; Mass: m/z 875.3 [M⁺+1]
Compound No. 168: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-Furan-3-yl-imidazol-1-yl)-butyl)-imino)erythromycin A; Mass: m/z 833.5 [M⁺+1]
Compound No. 169: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-Pyrazol-1-yl-pyridin-3-yl)-butyl)-imino)erythromycin A; Mass: m/z 844.4 [M⁺+1]
Compound No. 170: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Pyridin-3-yl-pyrazol-1-yl)-butyl)-imino)erythromycin A; Mass: m/z 844.5 [M⁺+1]
Compound No. 171: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-Thiophen-2-yl-pyridin-3-yl)-butyl)-imino)erythromycin A; Mass: m/z 860.3 [M⁺+1]
Compound No. 172: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-Phenyl-thiophen-2-yl)-butyl)-imino)erythromycin A; Mass: m/z 859.4 [M⁺+1]
Compound No. 173: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(N-(4-Amino-butyl)-N-thiazol-2-yl-nicotinimido)]erythromycin A; Mass: m/z 890.09 [M⁺+1]
Compound No. 174: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(N-(4-Amino-butyl)-N-thiazol-2-yl-nicotinimido)]erythromycin A; Mass: m/z 904.4 [M⁺+1]
Compound No. 175: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[4-(6-Pyrrol-1-yl-pyridin-3-yl)-imidazol-1-yl]-butyl)-imino)erythromycin A; Mass: m/z 909.5 [M⁺+1]
Compound No. 176: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-Pyrrol-1-yl-pyridin-3-yl)-butyl)-imino)erythromycin A; Mass: m/z 834.4 [M⁺+1]
Compound No. 177: 2-∝-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-Pyrrol-1-yl-thiazol-5-yl)-butyl)-imino)erythromycin A; Mass: m/z 849.3 [M⁺+1]
Compound No. 178: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-Imidazol-1-yl-pyridin-3-yl)-butyl)-imino)erythromycin A; Mass: m/z 844.4 [M⁺+1]
Compound No. 179: 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-[4-(Tetrahydro-furan-2-yl)-pyridin-3-yl]-butyl)-imino)erythromycin A; Mass: m/z 848.4 [M⁺+1]
Compound No. 180: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-p-tolyl-imidazol-1-yl)-butyl)-imino)]erythromycin A; Mass: m/z 857.08[M⁺+1]
Compound No. 181: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-aminophenyl)-imidazol-1-yl)-butyl)-imino)]erythromycin A; Mass: m/z 858.07[M⁺+1]
Compound No. 182: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-methyl-3-aminophenyl)-imidazol-1-yl)-butyl)-imino)]erythromycin A; Mass: m/z 872.1[M⁺+1]
Compound No. 183: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(3-imidazol-1-yl)-phenyl)imidazol-1-yl)-butyl)-imino)]erythromycin A; Mass: m/z 909.12[M⁺+1]
Compound No. 184: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N,N-dicyclopropyl-amino)-phenyl)-imidazol-1-yl)-butyl)-imino)]erythromycin A; Mass: m/z 938.20[M⁺+1]
Compound No. 185: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N,N-dimethyl-amino)phenyl)-imidazol-1-yl)-butyl)-imino)]erythromycin A; Mass: m/z 886.12[M⁺+1]
Compound No. 186: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(3-(tetrazol-1yl)-phenyl)-imidazol-1-yl)-butyl)-imino)]erythromycin A; Mass: m/z 911.09[M⁺+1]
Compound No. 187: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(2-(pyrrol-1-yl)-thiozol-5-yl)-imidazol-1-yl)-butyl)-imino)]erythromycin A; Mass: m/z 915.04[M⁺+1]
Compound No. 188: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(3-trifluoromethyl-phenyl)-imidazol-1-yl)-butyl)-imino)]erythromycin A; Mass: m/z 857.08[M⁺+1]
Compound No. 189: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((N-(thiazol-2-yl)-benzimido)-butyl)-imino)]erythromycin A; Mass: m/z 903.4[M⁺+1]
Compound No. 190: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(2-amino-pyrimidin)-5-yl)-imidazol-1-yl)-butyl)-imino)]erythromycin A; Mass: m/z 680.04 [M⁺+1]
Compound No. 191: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(4-(pyrrol-1-yl)-phenyl)-imidazol-1-yl)-butyl)-imino)]erythromycin A; Mass: m/z 908.13 [M⁺+1]
Compound No. 192: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(3-(pyrrol-1-yl)-phenyl)-imidazol-1-yl)-butyl)-imino)]erythromycin A; Mass: m/z 908.13 [M⁺+1]
Compound No. 193: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-acetyl-amino)-phenyl)-imidazol-1-yl)-butyl)-imino)]erythromycin A; Mass: m/z 900.11[M⁺+1]
Compound No. 194: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-isopropyl-amino)-phenyl)-imidazol-1-yl)-butyl)-imino)]erythromycin A; Mass: m/z 900.15[M⁺+1]
Compound No. 195: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(3-nitrophenyl)-imidazol-1-yl)-butyl)-imino)]erythromycin A; Mass: m/z 888.05[M⁺+1]
Compound No. 196: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-benzoyl-amino)-phenyl)-imidazol-1-yl)-butyl)-imino)]erythromycin A; Mass: m/z 962.18[M⁺+1]
Compound No. 197: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(3,4-dimethyl-phenyl)-imidazol-1-yl)-butyl)-imino)]erythromycin A; Mass: m/z 871.11 [M⁺+1]
Compound No. 198: 5-O-(3′-N-desmethyl-3′-N-ethyl)-11,12-dideoxy-3-O-decladinosyl-6-O-methyl-3-oxo-12,11-[oxycarbonyl-(((4-(4-Pyridin-3-yl-thiazol-2-yl)-butyl)-imino)]erythromycin A; Mass: m/z 862.4 [M⁺+1]
Compound No. 199: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(4-fluorophenyl)-imidazol-1-yl)-butyl)-imino)]erythromycin A; Mass: m/z 861.05 [M⁺+1]
Compound No. 200: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((4-(4-methoxy-phenyl)-imidazol-1-yl)-butyl)-imino)]erythromycin A; Mass: m/z 873.08 [M⁺+1]
Compound No. 201: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((N-(benzthiazol-2-yl)-benzimido)-butyl)-imino)]erythromycin A; Mass: m/z 953.19 [M⁺+1]
Compound No. 202: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-((N-(thiazol-2-yl)-nicotinamido)-butyl)-imino)]erythromycin A; Mass: m/z 904.12 [M⁺+1]
Compound No. 203: 2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-methyl-amino)-phenyl)-imidazol-1-yl)-butyl)-imino)]erythromycin A; Mass: m/z 872.10 [M⁺+1]
Compound No. 204: 2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(4-(2-aminopyridyl)-imidazol-1-yl)-butyl)-imino)]erythromycin A; Mass: m/z 8590.6 [M⁺+1]
Compound No. 205: 2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(5-(2-aminopyridyl)-2H-tetrazol-5-yl)-butyl)-imino)]erythromycin A; Mass: m/z 861.03[M⁺+1]
Compound No. 206: 2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3,4-difluorophenyl)-imidazol-1-yl)-butyl)-imino)]erythromycin A; Mass: m/z 878.04 [M⁺+1]
Compound No. 207: 2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(4-(2-chloropyridyl)-imidazol-1-yl)-butyl)-imino)]erythromycin A; Mass: m/z 878.49 [M⁺+1]
Compound No. 208: 2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-(6-aminopyridyl)-imidazol-1-yl)-butyl)-imino)]erythromycin A; Mass: m/z 859.06 [M⁺+1]
Compound No. 209: 2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(5-(2-Chloro-pyridin-4-yl)-tetrazol-2-yl)-butyl)-imino)]erythromycin A; Mass: m/z 880.46[M⁺+1]

### Example 6

### Pharmacological Activity

Compounds described herein displayed antibacterial activity in vitro especially against strains that are resistant to macrolides either due to efflux (mef strains) or ribosomal modification (erm) strains. These compounds are useful in treating community acquired pneumonia, upper and lower respiratory tract infections, skin and soft tissue infections, hospital acquired lung infections, bone and joint infections, and other bacterial infections, for example, mastitis, catether infection, foreign body, prosthesis infections or peptic ulcer disease.

Minimum inhibitory concentration (MIC) has been an indicator of in vitro antibacterial activity widely used in the art.

### Procedure:

### Medium

a) Cation adjusted Mueller Hinton Agar (MHA-Difco)

### b) Trypticase Soya Agar (TSA)

### Inoculum Preparation

Cultures were streaked on TSA for aerobic cultures and MHA with 5% sheep blood for fastidious cultures. Aerobic cultures were incubated at 37° C. for about 18-24 hours. Fastidious cultures were incubated CO₂ incubation (5% CO₂) at 37° C. for about 18-24 hours. Three to four well-isolated colonies were taken and saline suspensions were prepared in sterile densimat tubes. The turbidity of the culture was adjusted to 0.5-0.7 Mc Farland standard (1.5×10⁸ CFU/mL). The cultures were diluted 10 fold in saline to obtain inoculum sizes of approximately 1-2×10⁷ organisms/mL.

### Preparation of Drug Concentration

1 mg/mL concentration of stock solution of drugs was prepared in dimethylsulfoxide/distilled water/solvent given in National Committee for Clinical Laboratory Standards (NCCLS) manual. Serial two-fold dilutions of the compounds and standard drugs were prepared as per NCCLS manual.

The stock solution was changed according to the need of the experiment.

### Preparation of Agar Plates

Two mL of respective drug concentration was added to 18 mL of Molten Mueller Hinton agar to achieve the required range, for example 0.015 μg/mL-16 μg/mL. For fastidious cultures 1 mL of sheep blood was added in Molten Mueller Hinton agar.

MHA and MHA with 5% sheep blood plates without antibiotic for each set were prepared for controls. One MHA and MHA with 5% sheep blood plate without antibiotic for determining quality check for media was prepared.

### Preparation of Teflon Template

1 μL of each culture on each plate was replicated with the help of a replicator (i.e., Denley's multipoint replicator). The spots were allowed to dry and the plates were incubated for about 18-24 hours at 37° C. Fastidious cultures were incubated at 37° C. in a CO₂ incubator. The results were noted comparing with the control plates.

### Endpoint Definition

The concentration of drug at which there was complete disappearance of growth spot or formation of less than 10 colonies per spot was considered as Minimum Inhibitory Concentration (MIC).

The MICs of Quality Control (QC) strains were plotted on the QC chart for agar dilution method. If the MICs were within the range, the results interpreted by comparing MICs of standards against all organisms with those of test compounds.

### Precautions & Quality Control Measures

### Quality Control Strains

Staphylococcus aureus ATCC 29213
Enterococcus faecalis ATCC 29212
Eschericia coli ATCC 25922
Pseudomonas aeruginosa ATCC 27853
All 60 cultures were visually checked for purity.

Media Control: NCCLS disc diffusion assay using 10 μg discs of Gentamicin (Difco) against Pseudomonas aeruginosa ATCC 27853. A zone diameter of 16-21 mm was considered for optimum cation (Magnesium and Calcium) content of the media. The diameter was plotted in the media QC chart.

### REFERENCES

National Committee for Clinical Laboratory Standards (NCCLS), Methods for Dilution Antimicrobial Susceptibility Tests for Bacteria That Grow Aerobically—Fifth Edition; Approved Standard. M7-A5, Vol. 20. No. 2 (January 2000).
National Committee for Clinical Laboratory Standards, Performance Standards for Antimicrobial Susceptibility Testing—Twelfth informational supplement, M 100-12, Vol. 22 No. 1 (January 2002).

Results:
a) The compounds described herein exhibited MIC values against Staphylococcus aureus in the range of between about 0.03 μg/mL to about 8 μg/mL, for example between about 0.03 μg/mL to about 1 μg/mL.
b) The compounds described herein exhibited MIC values against sensitive Streptococcus pneumoniae in the range of between about 0.008 μg/mL to about 16 μg/mL, for example between about 0.008 μg/mL to about 0.5 μg/mL.
c) The compounds described herein exhibited MIC values against erythromycin resistant Streptococcus pneumoniae in the range of between about 0.06 μg/mL to about 16 μg/mL, for example between about 0.06 μg/mL to about 4 μg/mL.
d) The compounds described herein exhibited MIC values against Haemophilus influenzae in the range of between about 0.03 μg/mL to about 32 μg/mL, for example between about 0.03 μg/mL to about 16 μg/mL.
e) The compounds described herein exhibited MIC values against Moraxella species in the range of between about 0.004 μg/mL to about 4 μg/mL, for example between about 0.004 μg/mL to about 1 μg/mL.
f) The compounds described herein exhibited MIC values against telithromycin resistant Streptococcus pneumoniae in the range of between about 0.5 μg/mL to about 16 μg/mL, for example between about 0.5 μg/mL to about 16 μg/mL.
g) The compounds described herein exhibited MIC values against sensitive Streptococcus pyogenes in the range of between about 0.008 μg/mL to about 1 μg/mL, for example between about 0.008 μg/mL to about 0.15 μg/mL.
h) The compounds described herein exhibited MIC values against erythromycin resistant Streptococcus pyogenes in the range of between about 0.015 μg/mL to about 16 μg/mL, for example between about 0.015 μg/mL to about 16 μg/mL.
i) The compounds described herein exhibited MIC values against methicillin resistant Staphylococcus aureus to about 16 μg/mL.
j) The compounds described herein exhibited MIC values against sensitive Enterococci species in the range of between about 0.03 μg/mL to about μg/mL, for example between about 0.03 μg/mL to about 0.5 μg/mL.
k) The compounds described herein exhibited MIC values against resistant Enterococci species in the range of between about 1 μg/mL to about 16 μg/mL, for example between about 1 μg/mL to about 16 μg/mL.

We Claim:

1. A compound having the structure of Formula I, 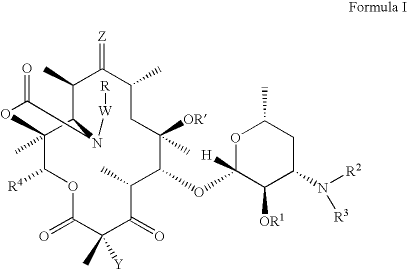
or a pharmaceutically acceptable salt, pharmaceutically acceptable solvate, enantiomer, diastereomer or polymorph thereof, wherein:
R¹ is hydrogen or a hydroxyl protecting group;
R² and R³ are independently alkyl, alkenyl, alkynyl, cycloalkyl, aryl, heterocycle, aralkyl, (heterocycle)alkyl or COR¹¹, wherein R¹¹ is hydrogen, alkyl or aralkyl, with the proviso that R² and R³ are not simultaneously methyl;
W is alkenyl, -G(CH₂)_{q}J-, —CR⁹R¹⁰, —NR⁹— or —SO₂, wherein
q is an integer of from 2 to 6;
G is no atom, —CO, —CS or —SO₂;
R⁹ and R¹⁰ are independently hydrogen or alkyl; and
J is no atom, —CR⁹R¹⁰ or N(R¹²)(CH₂)ₘ, wherein m is an integer of from 0 to 6; R⁹ and R¹⁰ are the same as defined earlier: and R¹² is hydrogen, alkyl, alkylene, alkynyl, COR⁸ or —(CH₂)ₘ—R⁸, wherein R⁸ is alkyl, aryl or heterocycle;
R is aryl or heterocycle;
R⁴ is alkyl, alkenyl or alkynyl;
R′ is alkyl or —(CH₂)ᵣ—U, wherein r is an integer of from 1 to 4 and U is alkenyl or alkynyl; and
Y is halogen, cyano or alkyl; Z is oxygen, sulfur or NOR¹¹, wherein R¹¹ is the same as defined earlier.

2. A compound of claim 1, wherein R is heterocycle; R² is methyl: R³ is alkyl (except methyl), alkenyl, cycloalkyl or COR¹¹; W is -G(CH₂)_{q}J- or CR⁹R¹⁰, wherein G, q, J, R⁹, R¹⁰ and R¹¹ are the same as defined in claim 1.

3. A compound of claim 1, wherein R¹ is hydrogen or a hydroxy protecting group (wherein the hydroxy protecting is benzoyl, tetrahydropyranyl or a trialkylsilylether); R² is CH₃; R³ is C₂H₅, —CH₂—CH═CH₂ or —CH₂CH₂F; W is —(CH₂)₄-J- wherein J is CH₂ or (CH₂)₀₋₁—N(CO)—Rₐ; and
R is 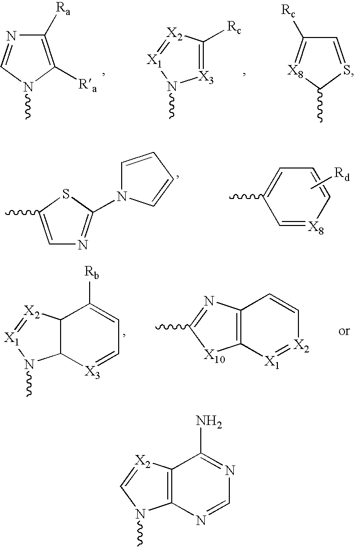
wherein
X₁₋X₃ are independently CH or N; X₄-X₈ are independently CH, CR₄ or N; X₉ is O, S, N, NH or CH; X₁₀ is NH or S; Rₐ is thienyl, furyl, pyrazolyl, oxazolyl, tetrazolyl, imidazolyl, pyridinyl, fluoropyridinyl, chloropyridinyl, aminopyridinyl, pyrazinyl, pyrimidinyl, aminopyrimidinyl, quinolinyl, pyrrolo-pyridyl, pyrrolo-thiazolyl or optionally substituted phenyl; R′ₐ is hydrogen or furyl; R_{b} is hydrogen or amino; R_{c} is hydrogen, thienyl, furyl, pyrazolyl, pyrazinyl, pyridinyl, aminopyridinyl, pyrimidinyl, pyrrolyl, imidazolyl or optionally substituted phenyl; and R_{d} is thienyl, pyrazolyl, imidazolyl, triazolyl, pyrrolyl or tetrahydrofuryl.

4. A compound selected from:
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((3-(imidazol-1-yl)-propyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(1H-imidazo[4,5-b]-pyridin-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-imidazo[4,5-b]-pyridin-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(imidazol-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((3-(imidazol-1-yl)propyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((3-(imidazol-1-yl)-propyl)-imino)]erythromycin A,
2-α-Fluoro-5-O-(3′-N-desmethyl-3′-N-propyl)-desosaminyl-11,12-dideoxy-3-O-decladinosyl-6-O-methyl-3-oxo-12,11-[oxycarbonyl-((3-(imidazol-1-yl)-propyl)-imino)]erythromycin A,
2-α-Fluoro-5-O-(3′-N-desmethyl-3′-N-propyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A.
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(pyrrolo-[2,3-b]pyridin-1-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(pyrrolo-[2,3-b]pyridin-1-yl)-butyl)imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3-N-n-propyl)-desosaminyl-11,12-dideoxy-3-O-decladinosyl-6-O-methyl-3-oxo-12,11-[oxycarbonyl-((4-(imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(pyrrolo-[2,3-b]pyridin-1-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(pyridin-[2,3-b]pyridin-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(purin-9-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(3H-imidazo[4,5-b]-pyridin-3-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyridin-3-yl)-imidazol-1-yl)-butyl)-imino)]erythromycin A,
2α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′-methyl-N′-quinolin-3-ylmethyl)-2-aminoethyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′-methyl-N′-pyridine-4-ylmethyl)-2-aminoethyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′-methyl-N′-pyridine-2-ylmethyl)-2-aminoethyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′-methyl-N′-pyridine-2-ylmethyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′-methyl-N′-pyridine-2-ylmethyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′-methyl-N′-pyridine-3-ylmethyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo 12,11-[oxycarbonyl-(((N′-methyl-N′-pyridine-4-ylmethyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′-methyl-N′-quinoline-4-ylmethyl)-2-aminoethyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′-pyridine-3-ylmethyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′-acetyl-N′-pyridine-3-ylmethyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′-pyridine-3-ylmethyl)-2-aminoethyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(((N′-acetyl-N′-pyridine-3-ylmethyl)-2-aminoethyl)-imino)]erythromycin A,
2-α-Fluoro-5-O-(3′-N-desmethyl-3-N-ethyl)-desosaminyl-11,12-dideoxy-3-O-decladinosyl-6-O-methyl-3-oxo-12,11-[oxycarbonyl-(((N′,N′-di-pyridine-3-yl-methyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-(((N′,N′-di-pyridine-3-yl-methyl)-2-aminoethyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(quinolin-8-yl)butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(pyridin-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(pyridin-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(pyridin-4-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(pyridin-4-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(pyridin-4-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(pyridin-4-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(pyridin-3-yl)-pentyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(pyridin-3-yl)-butyl)-imino)]erythromycin A, 2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(indol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-phenylimidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-nitroindol-1-yl)-butyl)imino)]erythromycin A,
2-α-Fluoro-5-O-(3-N-desmethyl-3′-N-ethyl)-desosaminyl-11,12-dideoxy-3-O-decladinosyl-6-O-methyl-3-oxo-12,11-[oxycarbonyl-((4-(1-imidazo[4,5-c]pyridin-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(benzotriazol-2-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(3-imidazo[4,5-c]pyridin-3-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(4-phenylimidazol-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(benzotriazol-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(benzotriazol-2-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(1H)-imdazo[4,5-b]pyridin-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(indol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(benzotriazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(3H-imidazo[4,5-b]pyridin-3-yl)-butyl)imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(4-phenylimidazol-1-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(benzotriazol-2-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(indol-1-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(3H-imidazo[4,5-b]pyridin-3-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(5-nitroindol-1-yl)-butyl)-imino)]erythromycin A,
2-α-Fluoro-5-O-(3′-N-desmethyl-3′-N-cyclopropyl)-desosaminyl-11,12-dideoxy-3-O-decladinosyl-6-O-methyl-3-oxo-12,11-[oxycarbonyl-((4-(5-nitroindol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-fluoroindol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(benzotriazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-methylbenzoimidazol-1-yl)-butyl)imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-ethylbenzoimidazol-1-yl)-butyl)-imino)]erythromycin A,
2-α-Fluoro-5-O-(3′-N-desmethyl-3′-N-isopropyl)-desosaminyl-11,12-dideoxy-3-O-decladinosyl-6-O-methyl-3-oxo-12,11-[oxycarbonyl-((4-(benzotriazol-1-yl)-butyl)-imino)]erythromycin A,
2-α-Fluoro-5-O-(3′-N-desmethyl-3′-N-allyl)desosaminyl-11,12-dideoxy-3-O-decladinosyl-6-O-methyl-3-oxo-12,11-[oxycarbonyl-((4-(2-ethylbenzoimidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-((4-pyridin-3-yl)-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(2-methylbenzoimidazol-1-yl)butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(1H-imidazo[4,5-b]pyridin-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(3H-imidazo[4,5-b]pyridin-3-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(5-nitroindol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(2-(pyridin-3-ylmethyl)-benzoimidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-(pyridin-3-yl-methyl)-benzoimidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(2-phenylimidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(5-fluoroindol-1-yl)-butyl)-imino)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((4-(5,6-dimethyl-benzoimidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5,6-dimethylbenzoimidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(3H-imidazo[4,5-c]pyridin-3-yl)-butyl)imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(2-methyl-3H-imidazo[4,5-b]pyridin-3-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-methyl-3H-imidazo[4,5-b]pyridin-3-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(1H-imidazo[4,5-c]pyridin-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-amino-9H-purin-9-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-phenylimidazol-1-yl)-butyl)-imino)]erythromycin A,
2-α-Fluoro-5-O-(3′-N-desmethyl-3-N-acetyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-cyclopropyl methyl)-desosaminyl-11,12-dideoxy-3-O-decladinosyl-6-O-methyl-3-oxo-12,11-[oxycarbonyl-((4-(3H-imidazo[4,5-b]pyridin-3-yl)-butyl)-imino)]erythromycin A,
2-α-Fluoro-5-O-(3′-N-desmethyl-3′-N-cyclopropyl methyl)-desosaminyl-11,12-dideoxy-3-O-decladinosyl-6-O-methyl-3-oxo-12,11-[oxycarbonyl-((4-(benzoimidazol-1-yl)-butyl)-imino)]erythromycin A,
2-α-Fluoro-5-O-(3′-N-desmethyl-3′-N-cyclopropyl methyl)-desosaminyl-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(benzotriazol-1-yl)-butyl) imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(4-phenyl-imidazol-1-yl)-butyl)imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(4-phenyl-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-trifluoromethyl-benzoimidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4,5-diphenyl-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4,5-diphenyl-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4-phenyl-imidazol-1-yl)-butyl)-imino)]erythromycin A,
2-α-Fluoro-5-O-(3′-N-desmethyl-3′-N-cyclopropyl methyl)-desosaminyl-11,12-dideoxy-3-O-decladinosyl-6-O-methyl-3-oxo-12,11-[oxycarbonyl-((4-(4-phenyl-imidazol-1-yl)-butyl)-imino)]erythromycin A,
2-α-Fluoro-5-O-(3′-N-desmethyl-3′-N-cyclopropyl methyl)-desosaminyl-11,12-dideoxy-3-O-decladinosyl-6-O-methyl-3-oxo-12,11-[oxycarbonyl-((4-(benzotriazol-1-yl)-butyl)-imino)]erythromycin A,
2-α-Fluoro-5-O-(3′-N-desmethyl-3′-N-cyclopropyl methyl)-desosaminyl-11,12-dideoxy-3-O-decladinosyl-6-O-methyl-3-oxo-12,11-[oxycarbonyl-((4-(4-(pyridin-3-yl)-imidazol-1-yl)-butyl)-imino)]erythromycin A,
2-α-Fluoro-5-O-(3′-N-desmethyl-3′-N-acetyl)-desosaminyl-11,12-dideoxy-3-O-decladinosyl-6-O-methyl-3-oxo-12,11-[oxycarbonyl-((4-(pyrrolo[2,3-b]pyridin-1-yl)-butyl)-imino)]erythromycin A,
2-α-Fluoro-5-O-(3′-N-desmethyl-3′-N-acetyl)-desosaminyl-11,12-dideoxy-3-O-decladinosyl-6-O-methyl-3-oxo-12,11-[oxycarbonyl-((4-(4-phenyl-imidazol-1-yl)-butyl)-imino)]erythromycin A,
2-α-Fluoro-5-O-(3′-N-desmethyl-3′-N-acetyl)-desosaminyl-11,12-dideoxy-3-O-decladinosyl-6-O-methyl-3-oxo-12,11-[oxycarbonyl-((4-(1H-imidazo[4,5-b]pyridin-1-yl)-butyl)-imino)]erythromycin A,
2-α-Fluoro-5-O-(3′-N-desmethyl-3′-N-acetyl)-desosaminyl-11,12-dideoxy-3-O-decladinosyl-6-O-methyl-3-oxo-12,11-[oxycarbonyl-((4-(3H-imidazo[4,5-b]pyridin-3-yl)-butyl)imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(3,5-diphenyl)-pyrazol-1-yl)-butyl)imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(4-(pyridin-3-yl)-imidazol-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl((4-(1H-imidazo[4,5-b]pyridin-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3,5-diphenyl)-pyrazol-1-yl)-butyl)-imino)]erythromycin A,
2-α-Fluoro-5-O-(3′-N-desmethyl-3′-N-cyclopropyl methyl)-desosaminyl-11,12-dideoxy-3-O-decladinosyl-6-O-methyl-3-oxo-12,11-[oxycarbonyl-((4-(1H-imidazo[4,5-b]pyridin-1-yl)-butyl)-imino)]erythromycin A.
2-α-Fluoro-5-O-(3′-N-desmethyl-3′-N-acetyl)-desosaminyl-11,12-dideoxy-3-O-decladinosyl-6-O-methyl-3-oxo-12,11-[oxycarbonyl-((4-(4-(pyridin-3-yl)-imidazo-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(4-(2,4-difluoro-phenyl)-imidazol-1-yl)-butyl)-imino)]erythromycin A,
2-α-Fluoro-5-O-(3′-N-desmethyl-3′-N-2-ethyl)-desosaminyl-11,12-dideoxy-3-O-decladinosyl-6-O-methyl-3-oxo-12,11-[oxycarbonyl-((4-(4-(2,4-difluorophenyl)-imidazol-1-yl)-butyl)-imino)]erythromycin A,
2-α-Fluoro-5-O-(3′-N-desmethyl-3′-N-2-allyl)-desosaminyl-11,12-dideoxy-3-O-decladinosyl-6-O-methyl-3-oxo-12,11-[oxycarbonyl-((4-(4-(2,4-difluoro-phenyl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl((4-(4-(furan-3-yl)-imidazol-1-yl)-butyl)-imino)]erythromycin A,
2-α-Fluoro-5-O-(3′N-desmethyl-3′-N-ethyl)-desosaminyl-11,12-dideoxy-3-O-decladinosyl-6-O-methyl-3-oxo-12,11-[oxycarbonyl-((4-(4-(furan-3-yl)-imidazol-1-yl)-butyl)-imino)]erythromycin A hydrochloride salt,
2-α-Fluoro-5-O-(3′N-desmethyl-3′-N-allyl)-desosaminyl-11,12-dideoxy-3-O-decladinosyl-6-O-methyl-3-oxo-12,11-[oxycarbonyl-((4-((4-phenyl-2H-pyrazol-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(pyridin-3-yl)-1H-imidazol-1-yl)pentyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyridin-4-yl)-1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4-(pyridin-4-yl)-1H-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-phenyl-2H-pyrazol-1-yl)-butyl)-imino)erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(1H-indazol-2-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(1H-indazol-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyridin-3-yl)-2H-pyrazol-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(1H-indazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyrazin-2-yl)-1H-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo 12,11-[oxycarbonyl-((4-(4-(thiophen-3-yl)-1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4-(thiophen-3-yl)-1H-imidazol-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(quinolin-3-yl)-1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(1H-indazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-[12,11-[oxycarbonyl-((4-(4-(pyrimin-5-yl)-1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4-(pyrimin-5-yl)-1H-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(furan-2-yl)-1H-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(thiophen-2-yl)-1H-imidazol-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(4-chloro-phenyl)-1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4-(4-chloro-phenyl)-1H-imidazol-1-yl)butyl)imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyridin-2-yl)-1H-imidazol-1-yl)-butyl)-imino)]erythromycin A,
2-α-Fluoro-5-O-(3′-N-desmethyl-3′-N-allyl)-desosaminyl-11,12-dideoxy-3-O-decladinosyl-6-O-methyl-3-oxo-12,11-[oxycarbonyl-((4-(4-(pyridin-2-yl)-1H-imidazol-1-yl)-butyl)imino)]erythromycin A,
2-α-Fluoro-5-O-(3′-N-desmethyl-3′-N-allyl)desosaminyl-11,12-dideoxy-3-O-decladinosyl-6-O-methyl-3-oxo-12,11-[oxycarbonyl-((9-(4-amino-butyl)-9H-purin-6-yl)-imino)]erythromycin A,
2-α-Fluoro-5-O-(3′-N-desmethyl-3′-N-2-allyl)-desosaminyl-11,12-dideoxy-3-O-decladinosyl-6-O-methyl-3-oxo-12,11-[oxycarbonyl-((4-(4-(pyridin-3-yl)-1H-pyrazol-1-yl)-butyl)imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(4-(4-chloro-phenyl)-1H-imidazol-1-yl)-butyl)-imino)]erythromycin A,
2-α-Fluoro-5-O-(3′-N-desmethyl-3′-N-2-fluoroethyl)-desosaminyl-11,12-dideoxy-3-O-decladinosyl-6-O-methyl-3-oxo-12,11-[oxycarbonyl-((4-(4-(pyridin-2-yl) 1H-imidazol-1-yl)butyl)imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-imidazo[4,5-b]pyridine-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(tetrazol-1-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(1H-benzoimidazol-2-yl) butyl)imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(6-fluoro-pyridin-3-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3H-imidazo[4,5-c]pyridine-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-([1,2,4]trizol-1-yl) phenyl)-butyl)-imino)]erythromycin A.
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(imidazol-1-yl)phenyl) butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(6-chloro-pyridin-3-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(3-(1-(4-aminobutyl)-1H-imidazol-4-yl)-phenyl)imino]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(pyridin-3-yl)-thiazol-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(3-(2-(4-aminobutyl)-thiazol-4-yl)-phenyl)imino]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyrazol-1-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-([1,4′]-bipyrazolyl-1′-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(imidazol-1-yl)-pyrazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyrazol-1-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-([3,3′]bithiophenyl-5-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-([2,3′]bithiophenyl-5′-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(furan-2-yl)-thiophen-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(oxazol-5-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(3-(pyrrol-1-yl)-[1,2,4]triazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-(thiophen-2-yl)-tetrazol-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl((4-(4-(thiophen-3-yl)-pyrazol-1-yl)-butyl)imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(furan-3-yl)-pyrazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(furan-2-yl)-pyrazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-phenyl-tetrazol-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-(4-methoxy-phenyl)-tetrazol-2-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-(furan-3-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-pyrazol-1-yl-pyridin-3-yl)butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(pyridin-3-yl)-pyrazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(5-thiophen-2-yl-pyridin-3-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo 12,11-[oxycarbonyl-((4-(4-phenyl-thiophen-2-yl)-butyl)imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-(N-(4-amino-butyl)-N-thiazol-2-yl-nicotinimido)]erythromycin A,
2-α-Fluoro-5-O-(3′-N-desmethyl-3′-N-ethyl)-desosaminyl 11,12-dideoxy-3-O-decladinosyl-6-O-methyl-3-oxo-12,11-[oxycarbonyl-(N-(4-amino-butyl)-N-thiazol-2-yl-nicotinimido)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(6-pyrrol-1-yl-pyridin-3-yl)-imidazol-1-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-pyrrol-1-yl-pyridin-3-yl)-butyl)-imino)]erythromycin A,
2-∝-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(2-pyrrol-1-yl-thiazol-5-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(6-imidazol-1-yl-pyridin-3-yl)-butyl)-imino)]erythromycin A,
2-α-Fluoro-5-O-(3′-N-desmethyl-3′-N-ethyl)-desosaminyl-11,12-dideoxy-3-O-decladinosyl-6-O-methyl-3-oxo-12,11-[oxycarbonyl-((4-(4-(tetrahydro-furan-2-yl)-pyridin-3-yl]-butyl)imino]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-tolyl-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-aminophenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-methyl-3-aminophenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(imidazol-1-yl)-phenyl)imidazol-1-yl) butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N,N-dicyclopropyl-amino)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N,N-dimethyl-amino)phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl((4-(4-(3-(tetrazol-1yl)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(2-(pyrrol-1yl)-thiozol-5-yl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-trifluoromethyl-phenyl)imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(N-(thiazol-2-yl)-benzimido)-butyl)-imino)]erythromycin A,
2-α-Fluoro-5-O-(3′-N-desmethyl-3′-N-ethyl) 11,12-dideoxy-3-O-decladinosyl-6-O-methyl-3-oxo-12,11-[oxycarbonyl-((4-(4-(2-amino-pyrimidin-5-yl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-(pyrrol-1yl)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(pyrrol-1yl)-phenyl)-imidazol-1-yl) butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-acetyl-amino)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-isopropyl-amino)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-nitrophenyl)imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-benzoyl-amino)-phenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3,4-dimethyl-phenyl)-imidazol-1-yl)-butyl)-imino)]erythromycin A,
5-O-(3′-N-desmethyl-3′-N-ethyl)-11,12-dideoxy-3-O-decladinosyl-6-O-methyl-3-oxo-12,11-[oxycarbonyl-((4-(4-Pyridin-3-yl-thiazol-2-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-fluorophenyl)-imidazol-1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(4-methoxy-phenyl)-imidazol 1-yl)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(N-(benzthiazol-2-yl)-benzimido)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(N-(thiazol-2-yl)-nicotinamido)-butyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((4-(4-(3-(N-methyl-amino)-phenyl)-imidazol-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(2-aminopyridin-4-yl)-imidazol-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(2-aminopyridin-5-yl)-2H-tetrazol-5-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3,4-difluorophenyl)-imidazol-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(2-chloropyridin-4-yl)-imidazol-1-yl)-butyl)-imino)]erythromycin A,
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(4-(6-aminopyridin-3-yl)-imidazol-1-yl)-butyl)-imino)]erythromycin A, or
2-α-Fluoro-5-O-(3′-N-desmethyl-3′-N-ethyl)desosaminyl-11,12-dideoxy-3-O-decladinosyl-6-O-methyl-3-oxo-12,11-[oxycarbonyl-((4-(5-(2-Chloro-pyridin-4-yl)-tetrazol-2-yl])-butyl)-imino)]erythromycin A,
or pharmaceutically acceptable salts, pharmaceutically acceptable solvates, enantiomers, diastereomers or polymorphs thereof.

5. A pharmaceutical composition comprising a therapeutically effective amount of one or more compounds of claim 1 together with one or more pharmaceutically acceptable carriers, excipients, or diluents.

6. A method for treating or preventing a condition caused by or contributed to by bacterial infection comprising administering to a mammal in need thereof a therapeutically effective amount of one or more compounds of claim 1.

7. The method of claim 6, wherein the condition is selected from community acquired pneumonia, upper or lower respiratory tract infections, skin or soft tissue infections, hospital acquired lung infections, hospital acquired bone or joint infections, mastitis, catether infection, foreign body, prosthesis infections or peptic ulcer disease.

8. The method of claim 6, wherein the bacterial infection is caused by Gram-positive, Gram-negative or anaerobic bacteria.

9. The method of claim 8, wherein the Gram-positive, Gram-negative or anaerobic bacteria is selected from Staphylococci, Streptococci Enterococci, Haemophilus, Moraxalla spp., Chlamydia spp., Mycoplasm, Legionella spp., Mycobacterium, Helicobacter, Clostridium. Bacteroides, Corynebacterium, Bacillus or Enterobactericeae.

10. The method of claim 9, wherein the bacterium is cocci.

11. The method of claim 10, wherein the cocci is drag resistant.

12. A process for preparing a compound of Formula XIII, 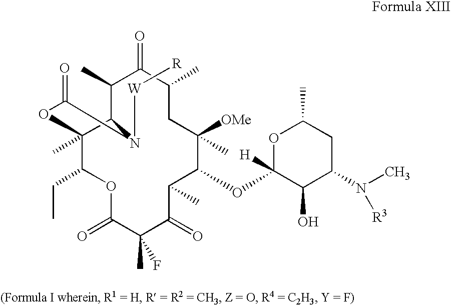
or a pharmaceutically acceptable salt, pharmaceutically acceptable solvate, prodrug, stereoisomer or polymorph thereof, wherein
R³ is alkyl, alkenyl, alkynyl, cycloalkyl, aryl, heterocycle. aralkyl, (heterocycle)alkyl or COR¹¹, wherein R¹¹ is hydrogen, alkyl or aralkyl, with the proviso that R³ is not methyl;
W is alkenyl, -G(CH₂)_{q}J-, —CR⁹R¹⁰, —NR⁹— or —SO₂, wherein
q is an integer of from 2 to 6;
G is no atom, —CO, —CS or —SO₂;
R⁹ and R¹⁰ are independently hydrogen or alkyl; and
J is no atom, —CR⁹R¹⁰ or N(R¹²)(CH₂)ₘ, wherein m is an integer of from 0 to 6; R⁹ and R¹⁰ are the same as defined earlier; and R¹² is hydrogen, alkyl, alkylene, alkynyl, COR⁸ or —(CH₂)ₘ—R⁸, wherein R⁸ is alkyl, aryl or heterocycle; and
R is aryl or heterocycle;
which comprises the steps of:
(a) hydrolyzing clarithromycin of Formula II, 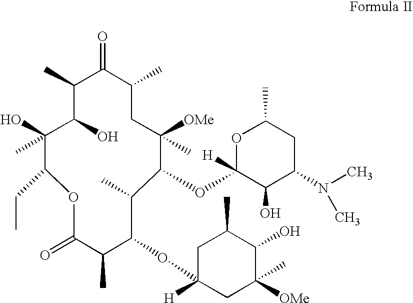
to form a compound of Formula III, 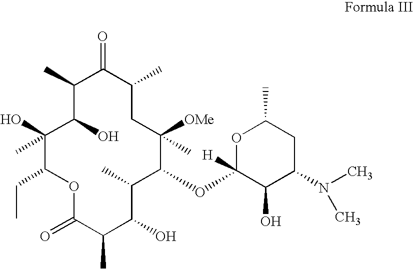
(b) protecting the compound of Formula III by reacting with one or more reagents of Formula R¹₂O or R¹X (wherein X is halogen) to form a compound of Formula IV, 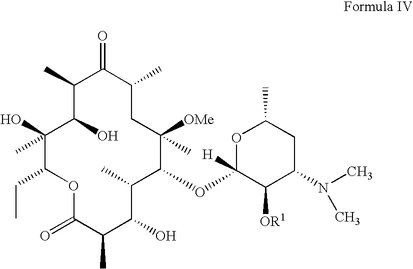
(c) desmethylating the compound of Formula IV at the 3′-N-dimethyl group to form a compound of Formula V, 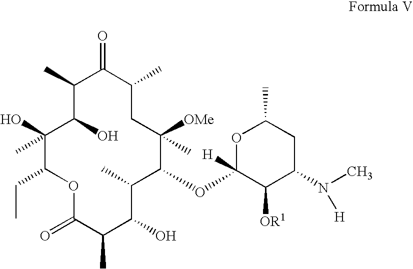
(d) alkylating the compound of Formula V with one or more reagents of Formula R³CHO, R₂³CO or R³X (wherein X is halogen) to form a compound of Formula VI (wherein R³ is the same as defined earlier), 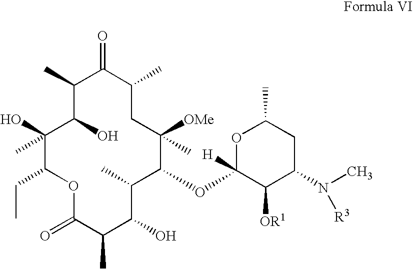
(e) converting the compound of Formula VI with one or more organic bases to form a compound of Formula VII, 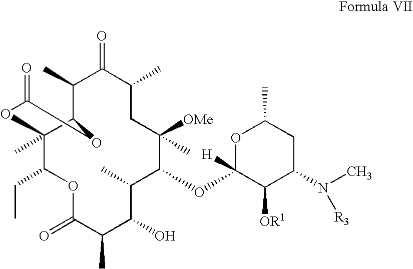
(f) reacting the compound of Formula VII with one or more organic bases to form a compound of Formula VIII 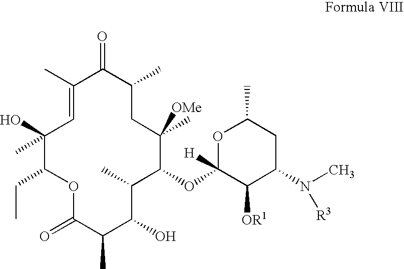
(g) oxidizing the compound of Formula VIII to form a compound of Formula IX, 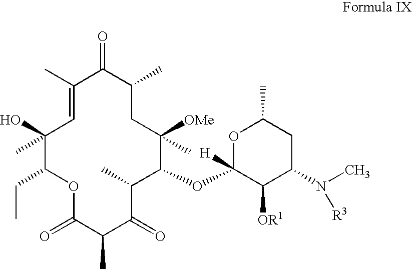
(h) reacting the compound of Formula IX with N,N′-carbonyldimidazole to form a compound of Formula X, 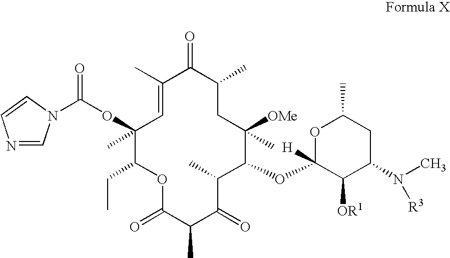
(i) reacting the compound of Formula X with a compound of Formula R—W—NF₂ to form a compound of Formula XI (wherein W and R are the same as defined earlier), 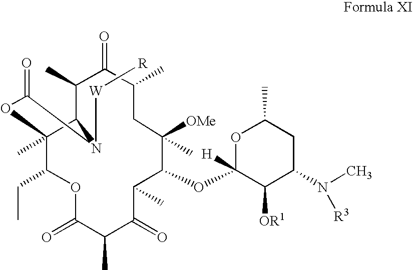
(j) fluoridating the compound of Formula XI to form a compound of Formula XII, and 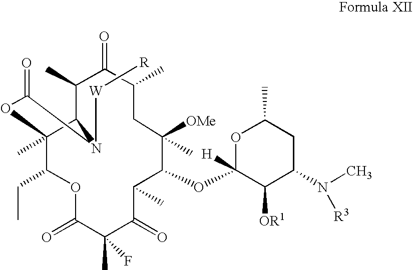
(k) deprotecting the compound of Formula XII to form a compound of Formula XIII.

13. A process for preparing a compound of Formula XIII, 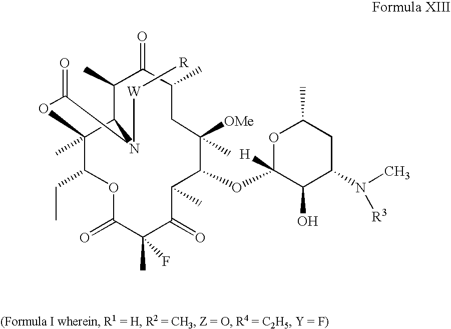
or a pharmaceutically acceptable salt, pharmaceutically acceptable solvate, prodrug, stereoisomer or polymorph thereof, wherein
R³ is alkyl, alkenyl, alkynyl, cycloalkyl, aryl, heterocycle, aralkyl, (heterocycle)alkyl or COR¹¹, wherein R¹¹ is hydrogen, alkyl or aralkyl, with the proviso that R³ is not methyl;
W is alkenyl, -G(CH₂)_{q}J-, —CR⁹R¹⁰, —NR⁹— or —SO₂, wherein
q is an integer of from 2 to 6;
G is no atom, —CO, —CS or —SO₂;
R⁹ and R¹⁰ are independently hydrogen or alkyl; and
J is no atom, —CR⁹R¹⁰ or N(R¹²)(CH₂)ₘ, wherein m is an integer of from 0 to 6; R⁹ and R¹⁰ are the same as defined earlier; and R¹² is hydrogen, alkyl, alkylene, alkynyl, COR⁸ or —(CH₂)ₘ—R⁸, wherein R⁸ is alkyl, aryl or heterocycle; and
R is aryl or heterocycle;
which comprises the steps of:
(a) reacting a compound of Formula IV with a suitable reagent selected from triphosgene, ethylene dicarbonate or a mixture thereof, 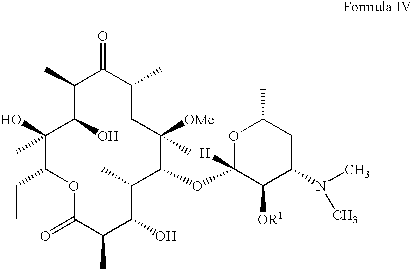
to form a compound of Formula XIV, 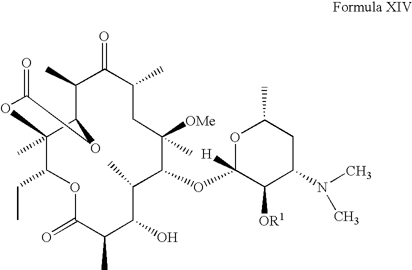
(b) reacting the compound of Formula XIV with one or more organic bases to form a compound of Formula XV, 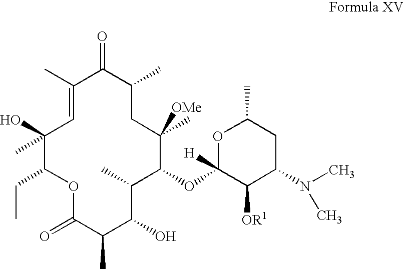
(c) oxidizing the compound of Formula XV to form a compound of Formula XVI, 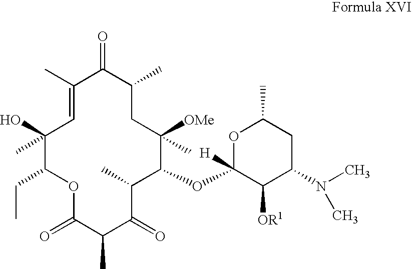
(d) desmethylating the compound of Formula XVI at the 3′-N-dimethyl group to form a compound of Formula XVII, 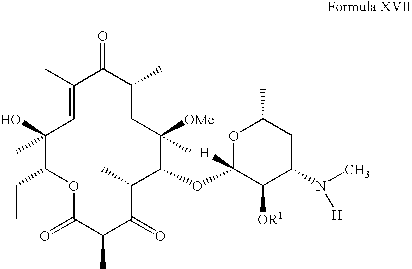
(e) alkylating the compound of Formula XVII with one or more reagents of Formula R³CHO, R³₂ CO or R³X (wherein X is halogen) to form a compound of Formula IX, 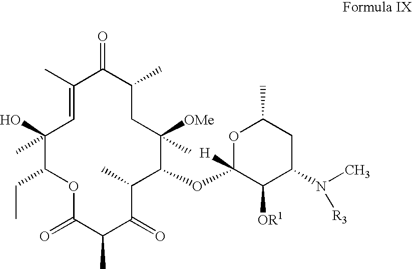
(f) fluorinating the compound of Formula IX to form a compound of Formula XVIII, 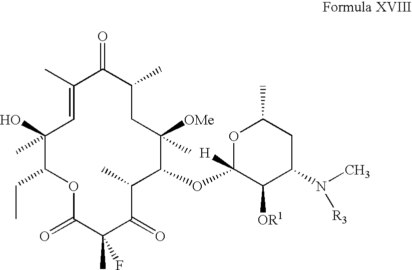
(g) reacting the compound of Formula XVIII with N,N′-carbonyldiimidazol to form a compound of Formula XIX, 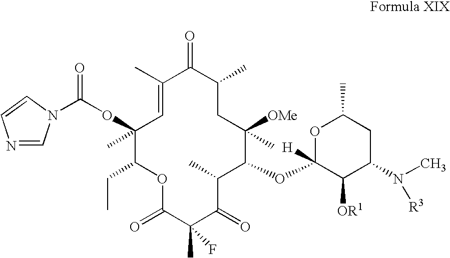
(h) reacting the compound of Formula XIX with a compound of Formula R—W—NH₂ to form a compound of Formula XII, and 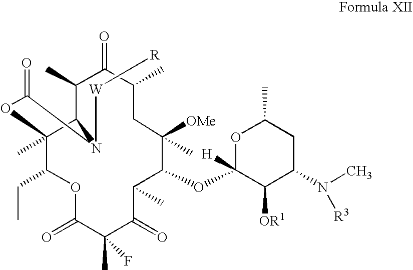
(i) deprotecting the compound of Formula XII to form a compound of Formula XIII,

14. A process for preparing a compound of Formula XIII, 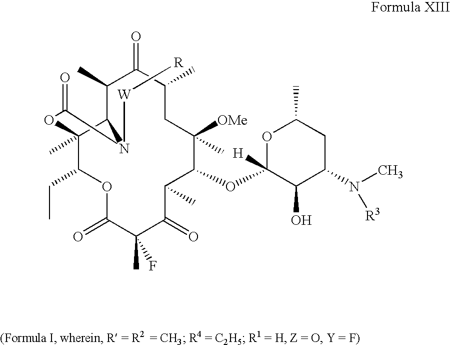
or a pharmaceutically acceptable salt, pharmaceutically acceptable solvate, stereoisomer, prodrug or polymorph thereof, wherein
R³ is alkyl, alkenyl, alkynyl, cycloalkyl, aryl, heterocycle, aralkyl, (heterocycle)alkyl or COR¹¹, wherein R¹¹ is hydrogen, alkyl or aralkyl, with the proviso that R³ is not methyl;
W is alkenyl, G(CH₂)_{q}J-, —CR⁹R¹⁰, —NR⁹— or —SO₂, wherein
q is an integer of from 2 to 6;
G is no atom, —CO, —CS or —SO₂;
R⁹ and R¹⁰ are independently hydrogen or alkyl; and
J is no atom, —CR⁹R¹⁰ or N(R¹²)(CH₂)ₘ, wherein m is an integer of from 0 to 6; R⁹ and R¹⁰ are the same as defined earlier; and R¹² is hydrogen, alkyl, alkylene, alkynyl, COR⁸ or —(CH₂)ₘ—R⁸, wherein R⁸ is alkyl, aryl or heterocycle; and
R is aryl or heterocycle;
which comprises the steps of:
(a) fluorinating the compound of Formula XVI, 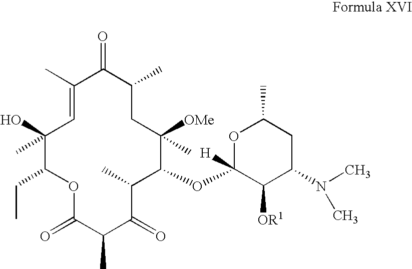
to form a compound of Formula XX, 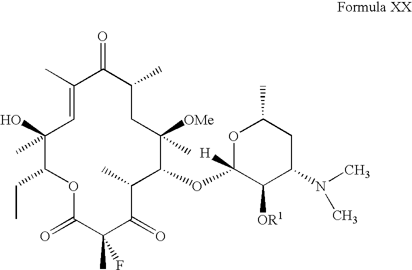
(b) reacting the compound of Formula XX with N,N′-carbonyldiimidazole to form a compound of Formula XXI, 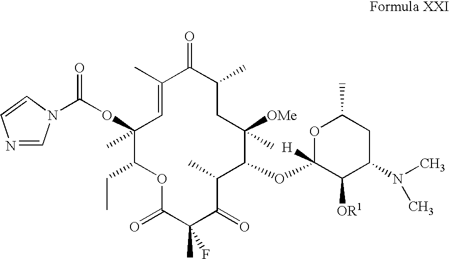
(c) reacting the compound of Formula XXI with a compound of Formula R—W—NH₂ to give a compound of Formula XXII, 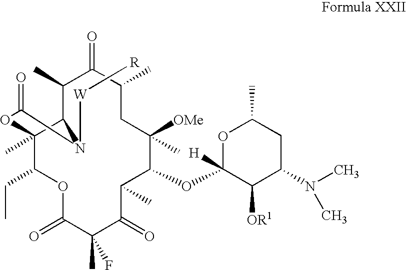
(d) deprotecting the compound of Formula XXII to form a compound of Formula XXIII, 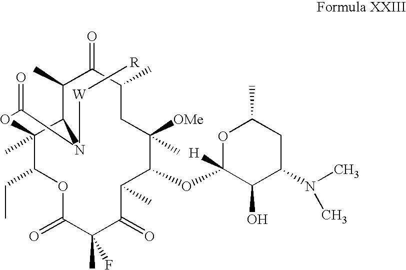
(e) desmethylating the compound of Formula XXIII at the 3′-N-dimethyl group to form a compound of Formula XXIV, and 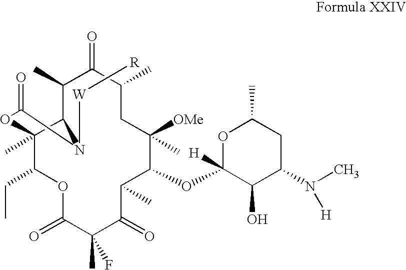
(f) alkylating the compound of Formula XXIV with one or more reagents of Formula R³CHO, R³₂CO or R³X (wherein X is halogen) to form a compound of Formula XIII.

15. A process for preparing a compound, of Formula XIII, 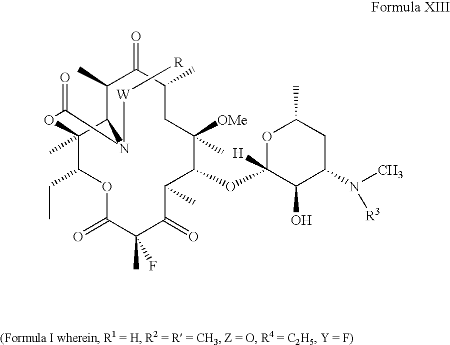
or its pharmaceutically acceptable salts, pharmaceutically acceptable solvates, stereoisomers, prodrugs or polymorphs, wherein
R³ is alkyl, alkenyl, alkynyl, cycloalkyl, aryl, heterocycle, aralkyl, (heterocycle)alkyl or COR¹¹, wherein R¹¹ is hydrogen, alkyl or aralkyl, with the proviso that R³ is not methyl;
W is alkenyl, -G(CH₂)_{q}J-, —CR⁹R¹⁰, —NR⁹— or —SO₂, wherein
q is an integer of from 2 to 6:
G is no atom, —CO, —CS or —SO₂;
R⁹ and R¹⁰ are independently hydrogen or alkyl; and
J is no atom, —CR⁹R¹⁰ or N(R¹²)(CH₂)ₘ, wherein m is an integer of from 0 to 6; R⁹ and R¹⁰ are the same as defined earlier; and R¹² is hydrogen, alkyl, alkylene, alkynyl, COR⁸ or (CH₂)ₘ—R⁸, wherein R⁸ is alkyl, aryl or heterocycle; and
R is aryl or heterocycle;
which comprises the steps of:
(a) deprotecting the compound of Formula XX (wherein R¹ is COPh), 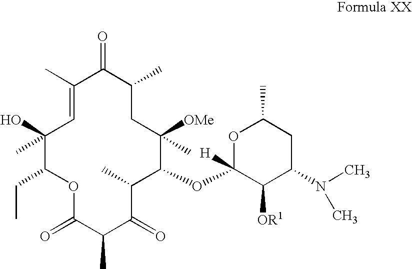
to form a compound of Formula XXV, 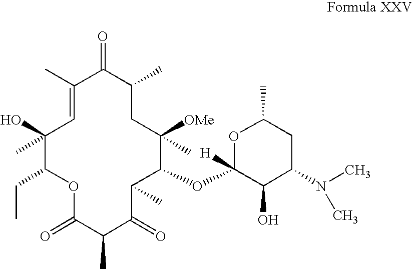
(b) desmethylating the compound of Formula XXV at 3-N′-dimethyl group to form a compound of Formula XXVI, 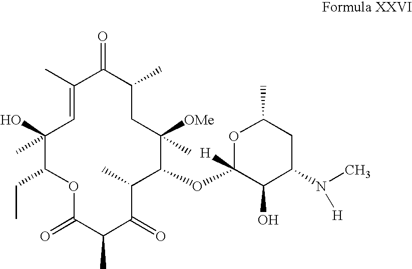
(c) alkylating the compound of Formula XXVI with one or more reagents of Formula R³CHO, R³₂CHO or R³X (wherein X is halogen) to form a compound of Formula XXVII, 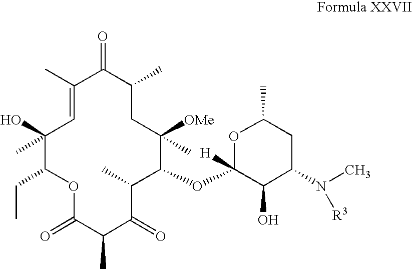
(d) protecting the compound of Formula XXVII with one or more reagents of Formula R¹₂O or R¹X (wherein X is halogen) to form a compound of Formula XVIII (wherein R¹ is COCH₃), 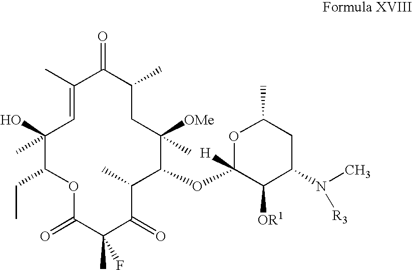
(e) reacting the compound of Formula XVII with N,N′-carbonyldiimidazole to form a compound of Formula XIX, 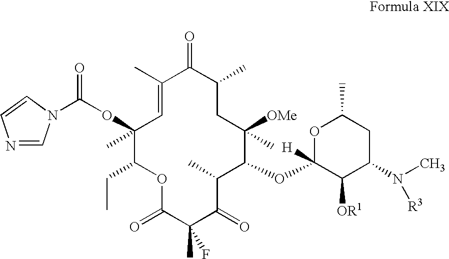
(f) reacting the compound of Formula XIX with a compound of Formula R—W—NH₂ to form a compound of Formula XII, and 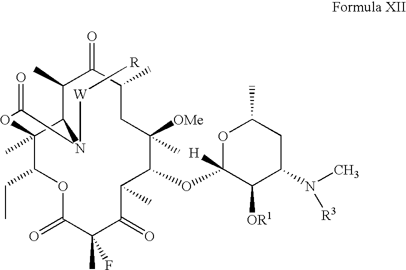
(g) deprotecting the compound of Formula XII to form a compound of Formula XIII.
